(19) United States
(12) Patent Application Publication
(10) Pub. No.: US 2006/0026700 A1
(43) Pub. Date: Feb. 2, 2006

(54) Tissue specific genes and gene clusters

(75) Inventors: Gilbert Jay, North Bethesda, MD (US); Richard Lebovitz, Arlington, VA (US); Youmin Shu, Potomac, MD (US); Zairen Sun, Rockville, MD (US); Meng Wu, Germantown, MD (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Origene Technologies, Inc., Rockville, MD (US)

(21) Appl. No.: 10/511,538

(22) PCT Filed: Apr. 15, 2003
(86) PCT No.: PCT/US03/11497

§ 371 Date: Aug. 18, 2005

Related U.S. Application Data
(60) Provisional application No. 60/372,669, filed on Apr. 16, 2002.
(60) Provisional application No. 60/374,823, filed on Apr. 24, 2002.
(60) Provisional application No. 60/376,558, filed on May 1, 2002.
(60) Provisional application No. 60/381,366, filed on May 20, 2002.
(60) Provisional application No. 60/403,648, filed on Aug. 16, 2002.
(60) Provisional application No. 60/411,882, filed on Sep. 20, 2002.
(60) Provisional application No. 60/424,336, filed on Nov. 7, 2002.

(51) Int. Cl. A01K 67/027; A61K 48/00; C12Q 1/68

(52) U.S. Cl. 800/14; 435/6; 514/44

(57) ABSTRACT

The present invention relates to genes and genes clusters which are expressed in a tissue specific maner. For example, the invention relates to a group of genes encoding GPCR-like receptors that are involved in the function and activity of the immune system. These genes are organized into a discrete cluster at chromosomal location 1q22 (the “immune gene complex”) and span about 700 kb of DNA. The region closest to the centromere comprises genes that are expressed predominantly in the thymus, while the distal region comprises genes which are expressed predominantly in the bone marrow and other hematopoietic cells. Another cluster of GPCR genes is located at chromosomal band 11q24. These genes are expressed predominantly in pancreatic tissue, establishing this region of chromosome 11 as a unique gene complex involved in pancreatic function. A cluster of transmembrane and GPCR-type receptor genes is also located at chromosomal band 11q12.2. These genes are expressed predominantly in the spleen (hence, “spleen gene” cluster), as well as other tissues of the immune and reticuloendothelial system (RES), indicating that establishing this region of the chromosome is involved in spleen, lymphoid, and/or reticuloendothelial function. Finally, genes coding for membrane proteins have been identified which are expressed selectively in bone marrow, kidney, pancreas, and retina.

This application claims the benefit of U.S. Application Ser. Nos. 60/372,669 Apr. 16, 2002, 60/374,823 filed Apr. 24, 2002, 60/376,558 filed May 1, 2002, 60/381,366 filed May 20, 2002, 60/403,648 filed Aug. 16, 2002, 60/411,882 filed Sep. 20, 2002, and 60/424,336 filed Nov. 7, 2002, which are hereby incorporated by reference in their entirety.

### DESCRIPTION OF THE DRAWINGS

FIGS. 1 and 2 show a physical map of the immune system gene complex. Sequence-tagged site (“STS”) markers are used to characterize the chromosomal regions. An STS is defined by two short synthetic sequences (typically 20 to 25 bases each) that have been designed from a region of sequence that appears as a single-copy in the human genome (the reference numbers, and the sequences which they represent, are hereby incorporated by reference in their entirety). These sequences can be used as primers in a polymerase chain reaction (PCR) assay to determine whether the site is present or absent from a DNA sample.

FIG. 3 shows the expression pattern of transmembrane proteins homologous to the olfactory G-protein-coupled receptor (“GPCR”) family in human tissues. To detect gene expression, PCR was carried out on aliquots of the normalized tissue samples using a forward and reverse gene-specific primers. Table 5 indicates the SEQ ID NO for each primer (“FOR” is the forward primer and “REV” is the reverse primer).

FIG. 4 shows the expression pattern of two olfactory G-protein-coupled receptor (“GPCR”) family members in human tissues. To detect gene expression, PCR was carried out on aliquots of the normalized tissue samples using a forward and reverse gene-specific primers. Table 6 indicates the SEQ ID NO for each primer (“FOR” is the forward primer and “REV” is the reverse primer).

FIGS. 5 (a and b) and 6 show the expression pattern in human tissues of genes selectively expressed in kidney tissue. To detect gene expression, PCR was carried out on aliquots of the normalized tissue samples using a forward and reverse gene-specific primers. Table 11 indicates the SEQ ID NO for each primer (“FOR”. is the forward primer and “REV” is the reverse primer).

FIG. 7 (a-b) show organization of pancreatic gene complex on chromosome 11q24.

FIG. 8 is a schematic drawing of five of the pancreatic olfactory G-protein-coupled receptor (“GPCR”) family members located in the gene complex showing regions of overlap. The numbering underneath the lines indicates amino acid position.

FIG. 9 (a and b) show the expression pattern of TMD0986, XM_{—}061780 (TMD0987), XM_{—}061781 (TMD0353), XM_{—}061784 (TMD0989), and XM_{—}061785 (TMD058) in human tissues. To detect gene expression, PCR was carried out on aliquots of the normalized tissue samples using a forward and reverse gene-specific primers. Table 12 indicates the SEQ ID NO for each primer (“FOR” is the forward primer and “REV” is the reverse primer).

FIG. 10 shows the expression pattern of TMD1030 (XM_{—}166853), TMD1029 (XM_{—}166854), TMD1028 (XM_{—}166855), and TMD0621 (XM_{—}166205) in human tissues. To detect gene expression, PCR was carried out on aliquots of the normalized tissue samples using a forward and reverse gene-specific primers. Table 17 indicates the SEQ ID NO for each primer (“F-oligo” is the forward primer and “R-oligo” is the reverse primer).

FIG. 11 shows the organization of the spleen gene complex on chromosome 11q12.2.

FIG. 12 (a-c) shows the expression of the pancreas genes in human tissues. To detect gene expression, PCR was carried out on aliquots of the normalized tissue samples using a forward and reverse gene-specific primers. Table 23 indicates the SEQ ID NO for each primer (“FOR” is the forward primer and “REV” is the reverse primer).

Expression patterns were analyzed as described below. A twenty-four tissue panel was used (lanes from left to right): 1, adrenal gland; 2, bone marrow; 3, brain; 4, colon; 5, heart; 6, intestine; 7, pancreas; 8, liver; 9, lung; 10, lymph node; 11, lymphocytes; 12, mammary gland; 13, muscle; 14, ovary, 15, pancreas; 16, pituitary; 17, prostate; 18, skin; 19, spleen; 20, stomach; 21, testis; 22, thymus; 23, thyroid; 24, uterus. The lane at the far left of each panel contains molecular weight standards. Polyadenylated mRNA was isolated from tissue samples, and used as a template for first-sand cDNA synthesis. The resulting cDNA samples were normalized using beta-actin as a standard. For the normalization procedure, PCR was performed on aliquots of the first-strand cDNA using beta-actin specific primers. The PCR products were visualized on an ethidium bromide stained agarose gel to estimate the quantity of beta-actin cDNA present in each sample. Based on these estimates, each sample was diluted with buffer until each contained the same quantity of beta-actin cDNA per unit volume. PCR was carried out using the primers described above, and reaction products were loaded on to an agarose (e.g., 1.5-2%) gel and separated electrophoretically.

### DESCRIPTION OF THE INVENTION

The present invention relates to tissue-selective genes and tissue-selective gene clusters. The polynucleotides and polypeptides are useful in variety of ways, including, but not limited to, as molecular markers, as drug targets, and for detecting, diagnosing, staging, monitoring, prognosticating, preventing or treating, determiniing predisposition to, etc., diseases and conditions, associated with genes of the present invention. The identification of specific genes, and groups of genes, expressed in pathways physiologically relevant to particular tissues, permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-. related methods, e.g., advertising, displaying, offering, selling, etc., such products for sale, commercial use, licensing, etc.

Immune Gene Complex

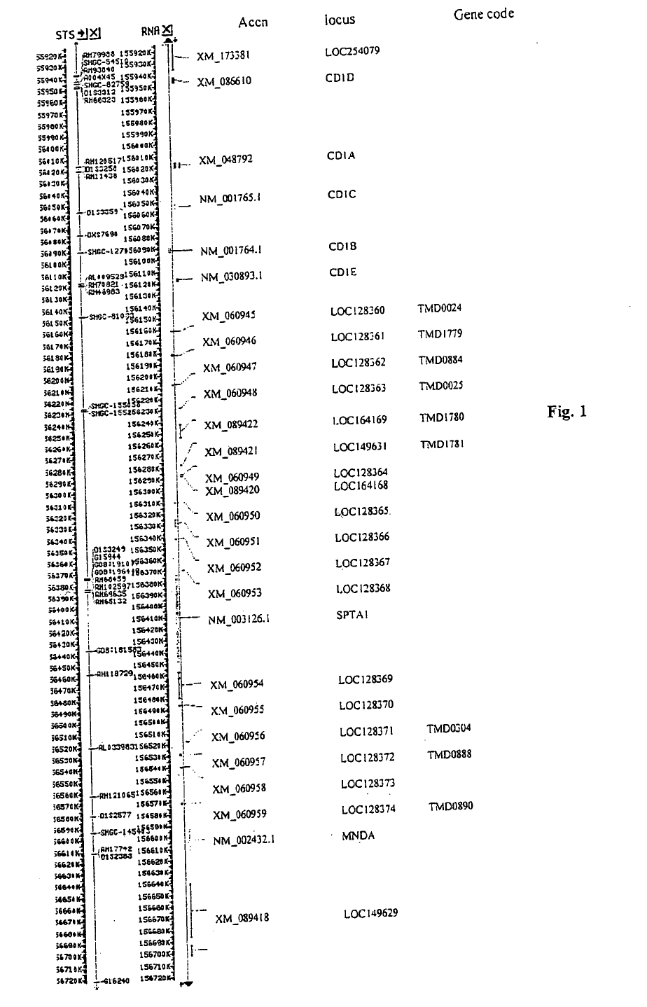

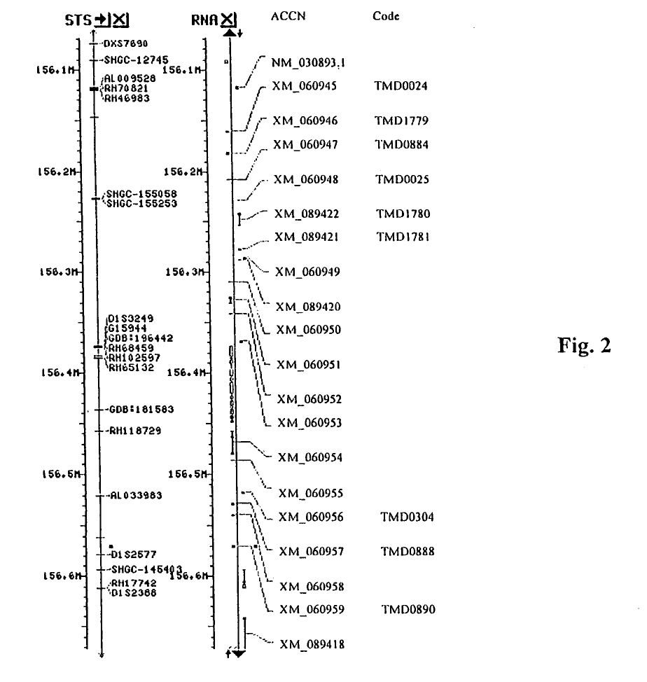

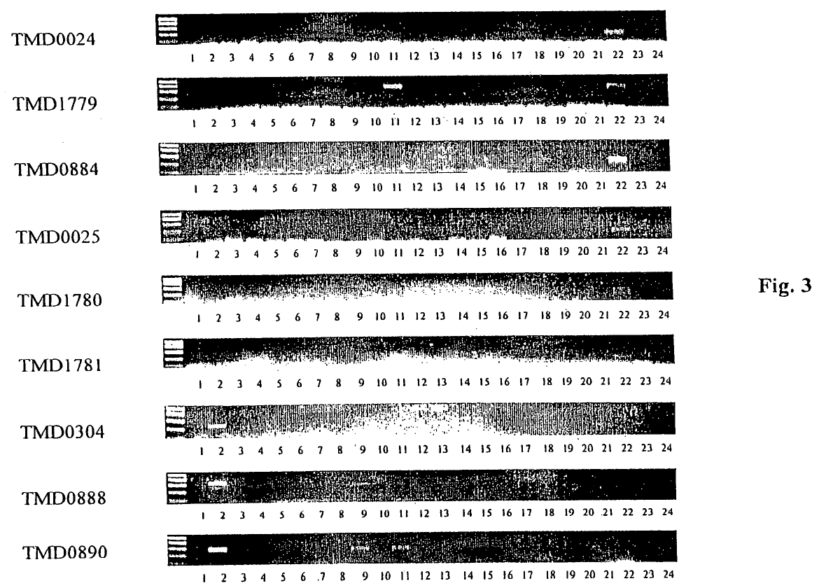

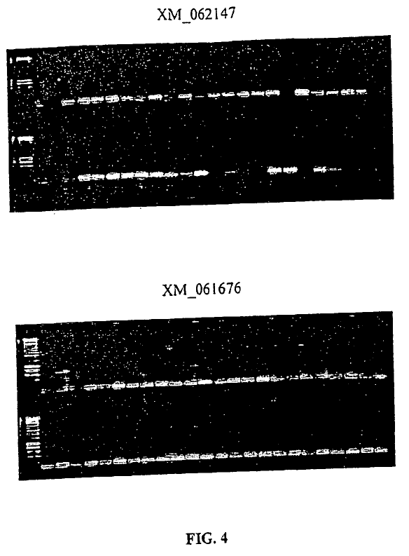

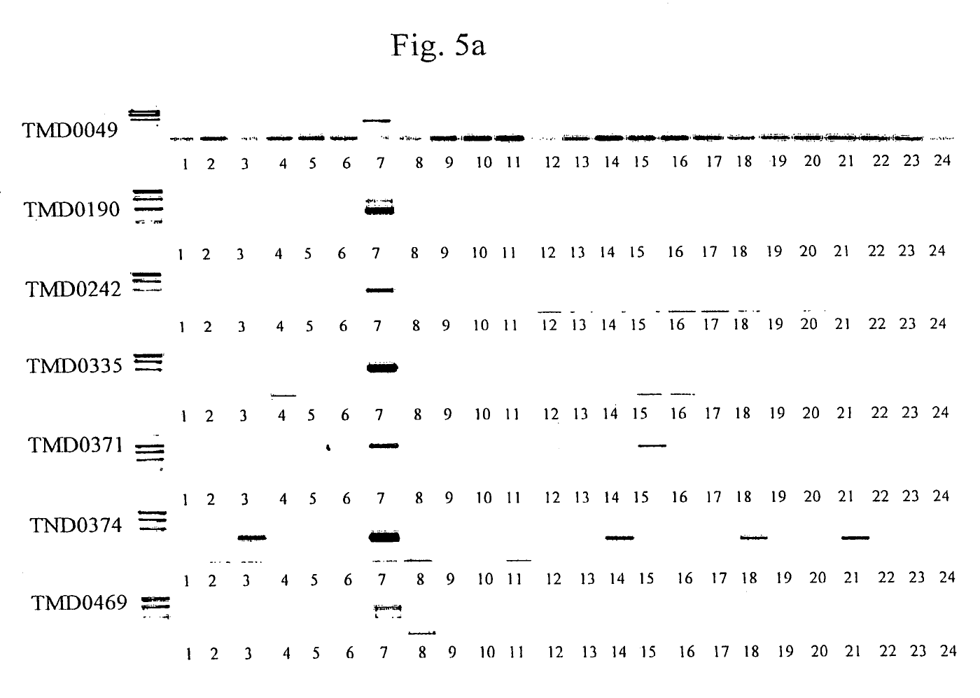

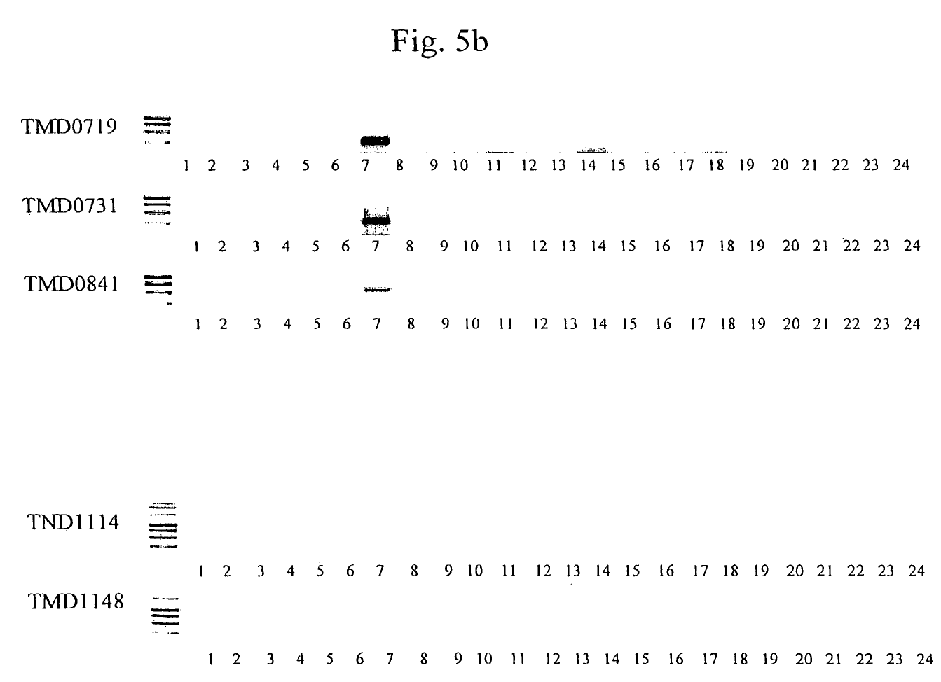

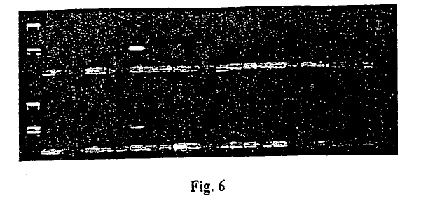

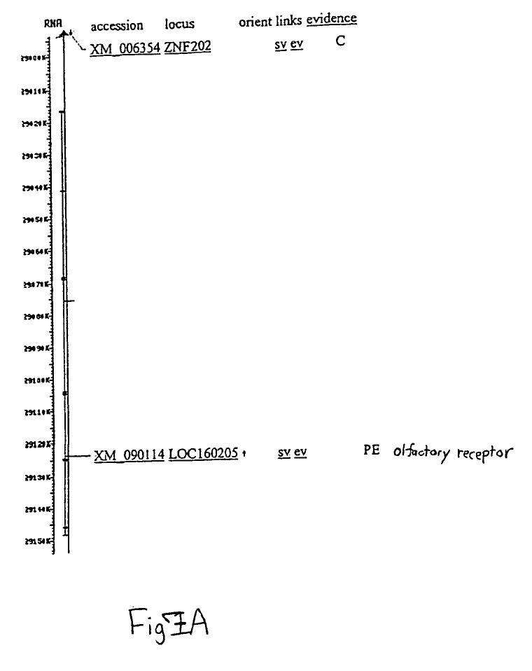

The present invention relates to a group of genes involved in the function and activity of the immune system. These genes are organized into a discrete cluster at chromosomal location 1q22 (the “immune gene complex”) and span hundreds of kb of DNA, e.g., about 700 kb of DNA. See, FIGS. 1 and 2. The region closest to the centromere comprises genes that are expressed predominantly in the thymus, while the distal region comprises genes which are expressed predominantly in the bone marrow and other hematopoietic cells.

The present invention relates to a composition consisting essentially of the 1q22 immune gene complex, comprising TMD0024 (XM_{—}060945), TID 1779 (XM_{—}060946), TMD0884 (XM_{—}060947), TMD0025 (XM_{—}060948), TMD1780 (XM_{—}089422), TMD1781 (XM_{—}089421), TMD0304 (XM_{—}060956), TMD0888 (XM_{—}060957), and TMD0890 (XM_{—}060959) genes, or a fragment thereof comprising at least two said genes. As discussed in more detail, the composition can comprise or consist essentially of the chromosome region between STS markers that define the genomic DNA, e.g., between SHGC-81033 and SHGC-145403, or a fragment thereof comprising at least two said genes.

The CD1 family, a cluster of genes previously identified as coding for proteins involved in antigen presentation (Sugita and Brenner, Seminars in Immunology, 12:511-516, 2000), are located at the proximal boundary of the immune gene complex. The expression of CD1a, b, and c genes are restricted to professional antigen-presenting cells, including dendritic cells and some B-cell subsets (Sugita and Brenner, ibid). CD1d is present on other cell types, in addition to hematopoietic cells, such as intestinal cells (Sugita and Brenner, ibid).

Adjacent to the CD1 family, is a cluster of genes coding for transmembrane proteins homologous to the olfactory G-protein-coupled receptor (“GPCR”) family. These genes include XM_{—}060945 (TMD0024), XM_{—}060346 (TMD1779), XM_{—}060947 (TMD0884), and XM_{—}060948 (TMD0025), and are expressed predominantly in thymus tissues (e.g., thyrnocytes). XM_{—}089421 (TMD1781) is also expressed in thymus, but it is present in much higher amounts in lymphocytes (“PBL”). This chromosomal region can be defined by STS markers, e.g., between SHGC-81033 and D1S3249, G15944, GDB:191077, GDB:196442, RH68459, RH102597, RH69635, or RH65132, or fragments thereof, such as fragments which comprise two or more genes.

The gene for human erythroid alpha spectrin (SPTA1) is distal to the GPCR thymus-restricted family. It is expressed in bone marrow cells, and is localized to the red cell membrane (Wilmotte et al., Blood, 90(10):4188-96, 1997). Next to it, is another cluster of genes coding for proteins that resemble the olfactory CPCR family. These include XM_{—}060956 (TMD0304), X_{—}060957 (TMD_{—}0888), and XM_{—}060959 (TMD089), and are expressed predominantly in the bone marrow, although other sites of expression are observed as well. See, e.g., Table 1. This chromosomal region can be defined by STS markers, e.g., between GDB:181583 or RH118729, and D1S2577 or SHGC-145403.

The gene for myeloid cell nuclear differentiation antigen (“MNDA” is next. MNDA is also expressed in bone marrow cells, particularly in normal and neoplastic myelomonocytic cells and a subset of normal and neoplastic B lymphocytes (Miranda et al., Hum. Pathol., 30(9): 1040-9, 1999).

The phrase “immune system” indicates any processes and cells which are involved in generating and carrying out an immune response. Immune system cells includes, but are not limited to, e.g., stem cells, pluripotent stem cell, myeloid progenitor, lymphoid progenitor, lymphocytes, B-lymphocytes, T-lymphocytes (e.g., naive, effector, memory, cytotoxic, etc.), thymocytes, natural killer, erythroid, megakaryocyte, basophil, eosinophil, granulocyte-monocyte, accessory cells (e.g., cells that participate in initiating lymphocyte responses to antigens), antigen-presenting cells (“APC”), mononuclear phagocytes, dendritic cells, macrophages, alveolar macrophages, etc., and any precursors, progenitors, or mature stages thereof

Table 1 is a summary of the genes and their expression patterns in accordance with the present invention. The genes and the polypeptides they encode can be used as diagnostic, prognostic, therapeutic, and research tools for any conditions, diseases, disorders, or applications associated with the tissues and cells in which they are expressed.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

In view of their selectivity and display on the cell surface, the olfactory GPCR family members of the present invention are a useful target for histological, diagnostic, and therapeutic applications relating to the cells in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. They can also be used to detect metastadic cells, in biopsies to identify bone marrow and thymus tissue, etc. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly as indicated in Table 2. Any of the methods described above and below can be accomplished in vivo, in vitro, or ex vivo (e.g., bone marrow cells or peripheral blood lymphocytes can be treated ex vivo and then returned to the body).

The expression patterns of the selectively expressed polynucleotides disclosed herein can be described as a “fingerprint” in that they are a distinctive pattern displayed by a tissue. Just as with a fingerprint, an expression pattern can be used as a unique identifier to characterize the status of a tissue sample. The list of expressed sequences disclosed herein provides an example of such a tissue expression profile. It can be used as a point of reference to compare and characterize samples. Tissue fingerprints can be used in many ways, e.g., to classify an unknown tissue, to determine the origin of metastatic cells, to assess the physiological status of a tissue, to determine the effect of a particular treatment regime on a tissue, to evaluate the toxicity of a compound on a tissue of interest, etc.

For example, the tissue-selective polynucleotides disclosed herein represent the configuration of genes expressed by a normal tissue. To determine the effect of a toxin on a tissue, a sample of tissue can be obtained prior to toxin exposure (“control”) and then at one or more time points after toxin exposure (“experimental”). An array of tissue-selective probes can be used to assess the expression patterns for both the control and experimental samples. As discussed in more detail below, any suitable method can be used. For instance, a DNA microarray can be prepared having a set of tissue-selective genes arranged on to a small surface area in fixed and addressable positions. RNA isolated from samples can be labeled using reverse transcriptase and radioactive nucleotides, hybridized to the array, and then expression levels determined using a detection system. Several kinds of information can be extracted: presence or absence of expression, and the corresponding expression levels. The normal tissue would be expected to express substantially all the genes represented by the tissue-selective probes. The various experimental conditions can be compared to it to determine whether a gene is expressed, and how its levels match up to the normal control.

Wile the expression profile of the complete gene set represented by the sequences disclosed here may be most informative, a fingerprint containing expression information from less than the full collection can be useful, as well. En the same way that an incomplete fingerprint may contain enough of the pattern of whorls, arches, loops, and ridges, to identify the individual, a cell expression fingerprint containing less than the full complement may be adequate to provide useful and unique identifying and other information about the sample. Moreover, because of heterogeneity of the population, as well differences in the particular physiological state of the tissue, a tissue's “normal” expression profile is expected to differ between samples, albeit in ways that do not change the overall expression pattern. As a result of these individual differences, each gene although expressed selectively in spleen, may not on its own 100% of the time be adequately enough expressed to distinguish said tissue. Thus, the genes can be used in any of the methods and processes mentioned above and below as a group, or one at a time.

Binding partners can also be used as to specifically deliver therapeutic agents to a tissue of interest. For example, a gene to be delivered to a tissue can be conjugated to a binding partner (directly or through a polymer, etc.), in liposomes comprising cell surface, and then administered as appropriate to the subject who is to be treated. Additionally, cytotoxic, cytostatic, and other therapeutic agents can be delivered specifically to the tissue to treat and/or prevent any of the conditions associated with the tissue of interest.

The present invention relates to methods of detecting immune system cells, comprising one or more of the following steps, e.g., contacting a sample comprising cells with a polynucleotide specific for a gene selected from Table 1, or a mammalian homolog thereof, under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID) NOS 3, 4, 8, 9, 14, 15, 22, 23, 27, 28, 35, 36, 42, 43, 49, 50, 57, and 58 (see, Table 5), and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting an immune system cell, comprising, one or more the following steps, e.g. contacting a sample comprising cells with a binding partner (e.g. an antibody, an Fab fragment, a single-chain antibody, an aptamer) specific for a polypeptide coded for by gene selected from Table 1, or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface as indicated in Table 2.

As indicated above, binding partners can be used to deliver agents specifically to the immune system, e.g., for diagnostic, therapeutic, and prognostic purposes. Methods of deliveringan agent to an immune cell can comprise, e.g., contacting an immune cell with an agent coupled to binding partner specific for a gene selected from Table 1 (i.e., TMD0024 (XM_{—}060945), TMD1779 (XM_{—}060946), TMD0884 (XM_{—}060947), TMD0025 (XM_{—}060948), TMD1780 (XM_{—}089422), TMD1781 (XM_{—}089421), TMD0304 (XM_{—}060956), TMD0888 (XM_{—}060957), and TMD0890 (XM_{—}060959)), whereby said agent is delivered to said cell. Any type of agent can be used, including, therapeutic and imaging agents. Contact with the immune system can be achieved in any effective manner, including by administering effective amounts of the agent to a host orally, parentally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos. 6,264,917, 6,254,852, 6,245,318, and 6,139,819. MR contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose.

The maturation of the immune system can also be modulated in accordance with the present invention, e.g., by methods of modulating the maturation of an immune system cell, comprising, e.g., contacting said cell with an agent effective to modulate a gene, or polypeptide encoded thereby, selected from Table 1, or a mammalian homolog thereof, whereby the maturation of an immune cell is modulated. Modulation as used throughout includes, e.g., stimulating, increasing, agonizing, activating, amplifying, blocking, inhibiting, reducing, antagonizing, preventing, decreasing, diminishing, etc.

The phrase “immune system cell maturation” includes indirect or direct effects on immune system cell maturation, i.e., where modulating the gene directly effects the maturational process by modulating a gene in a immune system cell, or less directly, e.g., where the gene is expressed in a cell-type that delivers a maturational signal to the immune system cell. Immune system maturation includes B-cell maturation, T-cell maturation, such as positive selection, negative selection, apoptosis, recombination, expression of T-cell receptor genes, CD4 and CDS receptors, antigen recognition, MHC recognition, tolerization, RAG expression, differentiation, TCR expression, antigen expression, etc. See also below and, e.g., Abbas et al., Cellular and Molecular Immunology, 4th Edition, W. B. Saunders Company, 2000, e.g., Pages 149-160. Process include reception of a signal, such as cytokinin or other GPCR ligand. Any suitable agent can be used, e.g., agents that block the maturation, such as an antibody to a GPCR of Table 1, or other GPCR antagonist.

The interactions between lymphoid and non-lymphoid immune system cells can also be modulated comprising, e.g., contacting said cells with an agent effective to modulate a gene, or polypeptide encoded thereby, selected from Table 1, or a mammalian homolog thereof, whereby the interaction is modulated. Lymphoid cells, includes, e.g., lymphocytes (T- and B-), natural killer cells, and other progeny of a lymphoid progenitor cell. Non-lymphoid cells include accessory cells, such as antigen presenting cells, macrophages, mononuclear phagocytes dendritic cells, non-lymphoid thymocytes, and other cell types which do not normally arise from lymphoid progenitors. Interactions that can be modulated included, e.g., antigen presentation, positive selection, negative selection, progenitor cell differentiation, antigen expression, tolerization, TCR expression, apoptosis. See, also above and below, for other immune system processes.

Promoter sequences obtained from GPCR genes of the present invention can be utilized to selectively express heterologous genes in immune system cells. Methods of expressing a heterologous polynucleotide in immune system cells can comprise, e.g., expressing a nucleic acid construct in immune system cells, said construct comprising a promoter sequence operably linked to said heterologous polynucleotide, wherein said promoter sequence is selected from Table 5. In addition to the cell lines mentioned below, the construct can be expressed in primary cells, such as thymocytes; bone marrow cells, stem cells, lymphoid progenitor cells, myeloid progenitor cells, monocytes, antigen presenting cells, macrophages, and cell lines derived therefrom, cell lines such as JHK3 (CRL-10991), KG-1 (CCL-246), KG-1a (CCL-246.1), U-937 (CRL-1593.2), VA-ES-BJ (CRL-2138), TUR (CRL-2367), ELI (CRL-9854), 28SC (CRL-9855), KMA (CRL-9856), THP-1 (TIB-2002), WEHI-274.1 (CRL-1679), M-NFS-60 (CRL-1838), MH-S (CRL-2019), SR4987 (CRL-2028),NCTC 3749 (CCL461), AMJ2-C8 (CRL 2455), AMJ2-C11 (CRL2456), PMJ2-PC (CRL-2457), EOC2 (CRL-2467), as well as any primary and established immune system cell lines.

Thymus

The thymus is the site of T-cell lymphocyte maturation. Immature lymphocytes migrate into the thymus from the bone marrow and other organs in which they are generated. The selection process that shape the antigen repertoire of T-cells takes place in the thymus organ. Both positive and negative selection processes take place. For a review, see, e.g., Abbas et al., Cellular and Molecular Immunology, 4th Edition, W. B. Saunders Company, 2000, e.g., Pages 126-130 and 149-160.

There are various diseases and disorders related to thymus tissue, including, but not limited to, thymic carcinoma, thymoma, Omenn syndrome, autoimmune diseases, allergy, Graves disease, Myasthenia gravis, thymic hyperplasia, DiGeorge syndrome, Good syndrome, promoting immune system regeneration after bone marrow transplantation, immuno-responsiveness, etc. The thymic selective genes and polypeptides encoded thereby can be use to treat or diagnose any thymic condition. For instance, chemotherapeutic and cytotoxic agents can be conjugated to thymic selective antibodies and used to ablate a thymoma or carcinoma. They can be used alone or in combination with other treatments. See, e.g., Graeber and Tamin, Serin. Thorac. Cardiovasc. Surg., 12:268-277, 2000; Loehrer, Ann. Med., 31 Suppl. 2:73-79, 1999.

Bone marrow

All circulating blood cells in the adult, including all immature lymphocytes, are produced in the bone marrow. In addition, the bone marrow is also the site of B-cell maturation. The marrow consists of a spongelike reticular framework located between long trabeculae. It is filled with fat cells, stromal cells, and precursor hematopoietic cells. The precursors mature and exit through the vascular sinuses

All the blood cells are believed to arise from a common stem cell. Lineages that develop from this common stem cell include, e.g., myeloid and lymphoid progenitor cells. The myeloid progenitor develops into, erytdrocytes (erythroid), platelets (megokaryocytic), basophils, eosinophils, granulocytes, neutrophils, and monocytes. The lymphoid progenitor is the precursor to B-lymphocytes, T-lymphocytes, and natural killer cells.

There are various diseases and disorders related to bone marrow, including, not limited to, e.g., red cell diseases, a plastic anemia (e.g., where there is a defect in the myeloid stem cell), pure red cell aplasia, white cell diseases, leukopenia, neutropenia, reactive (inflammatory) proliferation of white cells and nodes such as leukocytosis and lymphadenitis, neoplastic proliferation of white cells, malignant lymphoma, Non-Hodgkin's Lymphomas, Hodgkins disease, acute leukemias (e.g., acute lymphoblastic leukemia, acute myeloblastic leukemia, myelodysplatic snydrome), chromic myeloid leukemia, chronic leukemia. hairy cell leukemia, myeloproliferative disorders, plasma cell disorders, multiple myeloma, histiocytoses, etc.

Immune System Selective Genes

The present invention relates to genes involved in the function and activity of the immune system. XM_{—}062147 (TMD0088) and XM_{—}061676 (TMD0045) code for seven membrane spanning polypeptides which are homologous to members of the olfactory G-protein-coupled receptor (“GPCR”) family. XM_{—}062147 is expressed predominantly in bone marrow tissue, with no detectable expression in other tissues. XM_{—}061676 is also expressed predominantly in bone marrow tissue, but it is detected in peripheral blood lymphocytes, as well. As discussed in more detail below, XM_{—}062147 (TMD0088), XM_{—}061676 (TMD0045), and the polypeptides they encode, can be used as diagnostic, prognostic, therapeutic, and research tools for any conditions, diseases, disorders, or applications associated with the immune system and the cells in which they are expressed.

In view of their selectivity and display on the cell surface, the GPCR family members of the present invention are useful targets for histological, diagnostic, and therapeutic applications relating to the cells (e.g., B-cells and B-cell progenitors) in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. They can also be used to detect metastatic cells, in biopsies to identify bone marrow, lymphocytes, etc. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly as indicated in Table 2. Any of the methods described above and below can be accomplished in vivo, in vitro, or ex vivo (e.g., bone marrow cells or peripheral blood lymphocytes can be treated ex vivo and then returned to the body). Ex vivo methods can be used to eliminate cancerous cells from the bone marrow, to modulate bone marrow cells, to prime bone marrow cells for an immune response, to expand a particular class of cells expressing XM_{—}062147 (TMD0088) or XM_{—}061676 (TMD0045), to transfer genes into said cells (e.g., Banerjee and Bertino, Lancet Oncol., 3:154-158, 2002), etc.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

The phrase “immune system” indicates any processes and cells which are involved in generating and carrying out an immune response. Immune system cells includes, but are not limited to, e.g., stem cells, pluripotent stem cell, myeloid progenitor, lymphoid progenitor, lymphocytes, B-lymphocytes, T-lymphocytes (e.g., naive, effector, memory, cytotoxic, etc.), thymocytes, natural killer, erythroid, megakaryocyte, basophil, eosinophil, granulocyte-monocyte, accessory cells (e.g., cells that participate in initiating lymphocyte responses to antigens), antigen-presenting cells (“APC”), mononuclear phagocytes, dendritic cells, macrophages, etc., and any precursors, progenitors, or mature stages thereof.

XM_{—}062147 contains seven transmembrane segments. It is located on chromosomal band 11q12 within proximity to the locus for an inherited form of atopic hypersenstivity (OMIM 147050, e.g., associated with asthma, hay fever, and eczema). It has been suggested that the condition is a result of defect in the regulation of immunoglobulin E. XM_{—}061676 also is seven membrane spanning polypeptide. The chromosomal locus, 11p15, to which it maps is rich in genes associated with immune disorders, including Fanconi anemia, nucleoporin, mycloid leukemia, and T-cell lymphoblastic leukemia Arthroyosis multiplex congenita (distal type IIB) also maps closely to this chromosomal location.

The present invention relates to methods of detecting immune system cells, comprising one or more of the following steps, e.g., contacting a sample comprising cells with a polynucleotide specific for a gene selected from Table 6, or a mammalian homolog thereof, under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID NOS 67, 68, 76, and 77 (see, Table 6), and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting an immune system cell, comprising, one or more the following steps, e.g. contacting a sample comprising cells with a binding partner.(e.g. and antibody, an Fab fragment, a single-chain antibody, an aptamer) specific for a polypeptide coded for by gene selected from Table 6, or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface as indicated in Table 7.

As indicated above, binding partners can be used to deliver agents specifically to the immune system, e.g., for diagnostic, therapeutic, and prognostic purposes. Methods of delivering an agent to an immune cell can comprise, e.g., contacting an immune cell with an agent coupled to binding partner specific for a gene selected from Table 6, whereby said agent is delivered to said cell. Any type of agent can be used, including, therapeutic and imaging agents. Contact with the immune system can be achieved in any effective manner, including by administering effective amounts of the agent to a host orally, parentally, locally, systemically, intravenously, etc. The phrase “an agent coupled to binding partner” indicates that the agent is associated with the binding partner in such a-manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos. 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose.

The maturation of the immune system can also be modulated in accordance with the present invention, e.g., by methods of modulating the maturation of an immune system cell, comprising, e.g., contacting said cell with an agent effective to modulate a gene, or polypeptide encoded thereby, selected from Table 6, or a mammalian homolog thereof, whereby the maturation of an immune cell is modulated. Modulation as used throughout includes, e.g., stimulating, increasing, agonizing, activating, amplifying, blocking, inhibiting, reducing, antagonizing, preventing, decreasing, diminishing, etc.

The phrase “immune system cell maturation” includes indirect or direct effects on immune system cell maturation, i.e., where modulating the gene directly effects the maturational process by modulating a gene in a immune system cell, or less directly, e.g., where the gene is expressed in a cell-type that delivers a maturational signal to the immune system cell. Immune system maturation includes B-cell maturation, T-cell maturation, such as positive selection, negative selection, apoptosis, recombination, expression of T-cell receptor genes, CD4 and CD8 receptors, antigen recognition, MMC recognition, tolerization, RAG expression, differentiation, TCR expression, antigen expression, etc. See also below and, e.g., Abbas et al., Cellular and Molecular Immunology, 4th Edition, W. B. Saunders Company, 2000, e.g., Pages 149-160. Processes include reception of a signal, such as cytokinin or other GPCR ligand. Any suitable agent can be used, e.g., agents that block the maturation, such as an antibody to a GPCR of Table 6, or other GPCR antagonist.

The interactions between lymphoid and non-lymphoid immune system cells can also be modulated comprising, e.g., contacting said cells with an agent effective to modulate a gene, or polypeptide encoded thereby, selected from Table 6, or a mammalian homolog thereof, whereby the interaction is modulated. Lymphoid cells, includes, e.g., lymphocytes (T- and B-), natural killer cells, and other progeny of a lymphoid progenitor cell. Non-lymphoid cells include accessory cells, such as antigen presenting cells, macrophages, mononuclear phagocytes dendritic cells, non-lymphoid thymocytes, and other cell types which do not normally arise from lymphoid progenitors. Interactions that can be modulated included, e.g., antigen presentation, positive selection, negative selection, progenitor cell differentiation, antigen expression, tolerization, TCR expression, apoptosis. See, also above and below, for other immune system processes.

Promoter sequences obtained from GPCR genes of the present invention can be utilized to selectively express heterologous genes in immune system cells. Methods of expressing a heterologous polynucleotide in immune system cells can comprise, e.g., expressing a nucleic acid construct in immune system cells, said construct comprising a promoter sequence operably linked to said heterologous polynucleotide, wherein said promoter sequence is selected from Table 6. In addition to the cell lines mentioned below, the construct can be expressed in primary cells, such as thymocytes, bone marrow cells, stem cells, lymphoid progenitor cells, myeloid progenitor cells, monocytes, B-cells, antigen presenting cells, macrophages, and cell lines derived therefrom.

Kidney Selective Genes

The present invention relates to genes and polypeptides which are selectively expressed in kidney tissues: TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108). These genes and polypeptides are expressed predominantly in kidney tissues, making them, and the polypeptides they encode, useful as selective markers for kidney tissue and function, as well as diagnostic, prognostic, therapeutic, and research tools for any conditions, diseases, disorders, or applications associated with the kidney and the cells in which they are expressed. TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148. (XM_{—}087108), includes both human and mammalian homologs of it. SEQ ID NOS 78-103 represent pariticular alleles, but the present invention relates to other alleles, including naturally-occurring polymorphisms (i.e., a polymorphism in the nucleotide sequence which is identified in populations of mammals) and homologs thereof. More information on these genes is summarized in Tables 8-11.

In view of their selectivity and display on the cell surface, the polypeptides and polynucleotides of the present invention are useful targets for histological, diagnostic, and therapeutic applications relating to the cells (e.g., juxtaglomerular cells which secrete renin, peritubular cells, endothelial cells, e.g., of the cortex and outer medulla, mesangial cells which secrete inflammatory mediators including NO and products of cyclooxygenase, visceral epithelial cells, parietal epithelial cells, podocytes, early proximal tubule cells which secrete, e.g., angiotensin converting enzyme and neutral endopeptidase, late distal tubule cells that produce, e.g., prolyl endopeptidase, serine endopeptidase, carboxypeptidase, and neutral endopeptidase, renomedullary interstitial cells, etc) in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. They can also be used to detect metastatic cells, in biopsies, to identify kidney, etc. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly as indicated in Table 9. Any of the methods described above and below can be accomplished in vivo, in vitro, or ex vivo.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

The present invention relates to methods of detecting kidney cells, comprising one or more of the following steps, e.g., contacting a sample comprising cells with a polynucleotide specific for TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), or a mammalian homolog thereof; under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID NOS 104, 105, 107, 108, 111, 112, 115, 116, 119, 120, 122, 123, 126, 127, 131, 132, 135, 136, 138, 139, 142, 143, 145, 146, 149, 150, and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting a kidney cell, comprising, one or more the following steps, e.g. contacting a sample comprising cells with a binding partner (e.g. an antibody, an Fab fragment, a single-chain antibody, an aptamer) specific for a polypeptide coded for by TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface as indicated in Table 9.

As indicated above, binding partners can be used to deliver agents specifically to the kidney, e.g., for diagnostic, therapeutic, and prognostic purposes. Methods of delivering an agent to a kidney cell can comprise, e.g., contacting a kidney cell with an agent coupled to binding partner specific for TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), whereby said agent is delivered to said cell. Any type of agent can be used, including, therapeutic and imaging agents. Contact with the kidney can be achieved in any effective manner, including by administering effective amounts of the agent to a host orally, parentally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers. Any cell expressing a polypeptide coded for by TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), can be targeted, including, e.g., juxtaglomerular, peritubular, endothelial, mesangial, visceral epithelial, parietal epithelial, podocytes, early proximal tubule, late distal tubule, renomedullary interstitial, etc.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos, 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose.

A kidney cell (see above for examples of kidney cell types) can also be modulated in accordance with the present invention, e.g., by methods of modulating a kidney cell, comprising, e.g., contacting said cell with an agent effective to modulate TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), or the biological activity of a polypeptide encoded thereby, or a mammalian homolog thereof, whereby said kidney cell is modulated. Modulation as used throughout includes, e.g., stimulating, increasing, agonizing, activating, amplifying, blocking, inhibiting, reducing, antagonizing, preventing, decreasing, diminishing, etc.

An activity or function of the kidney cell can be modulated, including, e.g., glomerular filtration rate, filtration pressure, renal autoregulation (including via myogenic mechanism and tubuloglomerular feedback mechanism), tubular reabsorption, tubular secretion, and renal clearance. In addition, the transcription, translation, synthesis, degradation, expression, etc., of any secretory or polypeptide produced by a kidney cell can be modulated, including, but not limited to, renin-angiotensin activity, production and secretion of prostaglandins, nitric oxide, kallikrein, adenosine, endothelin, erythropoietin, and other hormones, enzymes, and other secretory and intracellular factors. The response of a kidney cell to stimuli can also be modulated, including, but not limited to, ligands to TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), oxygen levels, blood pressure, etc.

The present invention also relates to polypeptide detection methods for assessing kidney function, e.g., methods of assessing kidney function, comprising, detecting a polypeptide coded for by TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), fragments thereof, polymorphisms thereof, in a body fluid, whereby the level of said polypeptide in said fluid is a measure of kidney function. Kidney function tests are usually performed to determine whether the kidney is functioning normally as a way of diagnosing kidney disease. Various tests are commonly used, including, e.g., BUN (blood urea nitrogen), serum creatinine, estimated GFR, ability to concentrate urine, BUN/creatine ratio, urine sodium and other electrolytes, urine NAG (N-acetyl-beta-glucosaminidase, adenosine deaminase, urinary alkaline phosphatase, serum and urine beta-2-microglobulin, serum uric acid, isotope scans, Doppler sonogram, positron emission tomography, specific gravity of urine, microalbumin, total protein, etc. Detection of TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), provides an additional assessment tool, especially in diseases such as chromic renal failure, urinary tract infections, kidney stones, nephritic syndrome, nephritic syndrome, kidney disease due to diabetes or high blood pressure, etc., As with the other tests, elevated levels of said polypeptide in blood, or other fluids, can indicate impaired kidney function. Values can be determined routinely, as they are for other kidney function markers, such as those mentioned above. Detecting can be performed routinely (see below), e.g., using an antibody which is specific for said polypeptide, by RIA, ELISA, or Western blot, etc.

Promoter sequences obtained from genes of the present invention can be utilized to selectively express heterologous genes in kidney cells. Methods of expressing a heterologous polynucleotide in kidney cells can comprise, e.g., expressing a nucleic acid construct in kidney cells, said construct comprising a promoter sequence operably linked to said heterologous polynucleotide, wherein said promoter sequence is selected SEQ ID NOS 106, 109, 110, 113, 114, 117, 118, 121, 124, 125, 128-130, 133, 134, 137, 140, 141, 144, 147, 148, and 151. In addition to the cell lines mentioned below, the construct can be expressed in primary cells or in established cell lines.

Kidney

The kidney maintains the constancy of fluids in an organism's internal environment, and is therefore of great importance in maintaining health and vitality. Each day, the kidney filters the blood, removing and concentrating toxins, metabolic wastes, and excess ions, allowing them to be excreted by the body in the form of urine. The excretory function of the kidney is performed by over one million blood units called nephrons, each a miniature blood filtering and processing unit. A nephron consists of a glomerulus, a tuft of capillaries, and a renal tubule. In addition to their excretory function, kidneys produce a number of different hormones, enzymes, and other secreted molecules, including the enzyme renin and the hormone erythropoietin. The kidney also is responsible for metabolizing vitamin D into its active form, calcitriol. For a full description of the kidney's function and structure, see, e.g., Human Anatomy and Physiology, Marieb, E. N., 3^{rd} Edition, Benjamin/Cummings Publishing Company, Inc., 1995, pp 896-923.

The glomerulus is a high pressure capillary bed which filters out most substances smaller than large plasma proteins across the fenestrated glomerular epithelium, the intervening basement membrane, and the podocyte-containing visceral membrane of the glomerulus capsule. The external layer of the glomerulus is called the parietal layer, consisting predominantly of a squamous epithelium. This layer is structural. Underneath it, is the visceral layer which consists of the modified branching epithelial cells called podocytes. These sit on top of the fenestratrated glomerular endothelium. The glomerulus is connected to the renal tubule, a highly differentiated and long tube, having three major elements: the proximal convoluted tubule, the loop of Henel, and the distal convoluted tubule. Different regions of the tubule have different functions in absorption and secretion.

Renal cells produce a variety of different hormones and chemicals, including, prostaglandins, nitric oxide, kallilrein family, adenosine, endothelin family, renin, erythropoietin, aldosterone, antidiuretic hormone (vasopressin), natriuretic hormones, etc. Renin is involved in modulating blood pressure. It cleaves angiotensinogen, a plasma peptide, splitting off a fragment containing 10 amino acids called angiotensin I. Angiotensin I is cleaved by a peptidase secreted by blood vessels called angiotensin converting enzyme (ACE), producing angiotensin II, which contains 8 amino acids. Angiotensin II has many direct effects on blood pressure. Erythropoietin stimulates red blood cell production in the bone marrow.

TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), can be used to identify, detect, stage, determine the presence of, prognosticate, treat, study, etc., diseases and conditions of the kidney. These include, but are not limited to, diseases that affect the four basic morphologic components, glomeruli, tubules, interstitium, and blood vessels. Diseases include, e.g., acute nephritic syndrome, nephritic syndrome, renal failure, urinary tract infections, renal stones, cystic diseases of the kidney, e.g., cystic renal dysplasia, polycystic disease (autosomal dominant and recessive types), medullary cystic disease, acquired cystic disease, renal cysts, parenchymal cysts, perihilar renal cysts (pyelocalyceal cysts, hilar lymphangitic cysts), glomerular diseases, diseases of tubules, tubulointerstitial diseases, tumors of the kidney, such as benign tumors (cortical adenoma, renal fibroma, renomedullary interstitial cell tumor), malignant tumors (renal cell carcinoma, hypernephroma, adenocarcinoma of kidney, Wilms' tumor, nephroblastoma, urothelial carcinoma), renal coloboma, nephorblastoma, clear cell sarcoma of kidney (CCSK), rhabdoid tumor of kidney (RTK), von Hippel-Lindau disease, oncocytoid renal cell carcinoma (RCC), renal leiomyoblastoma, etc. TMD0049 (XM_{—}057351), TMD0190 (XM_{—}087157), TMD0242 (XM_{—}088369), TMD0335 (XM_{—}089960), TMD0371, TMD0374, TMD0469 (XM_{—}038736), TMD0719 (XM_{—}059548), TMD0731 (XM_{—}059703), TMD0785 (XM_{—}060310), TMD0841 (XM_{—}060623), TMD1114 (NM_{—}019841), and/or TMD 1148 (XM_{—}087108), can also be used for staging and classifying conditions and diseases of the present invention, alone, or in combination with conventional staging and classification schemes

Pancreatic Gene Complex

The present invention relates to a cluster of olfactory GPCR (G-protein coupled) receptor genes located at chromosomal band 11q24. These genes are expressed predominantly in pancreatic tissue, establishing this region of chromosome 11 as a unique gene complex involved in pancreatic function. See, Table 12. Because of their exquisite selectivity for pancreatic tissues, the pancreatic gene complex (“PGC”), and the genes which comprise it, are useful to assess pancreas tissue and function for diagnostic, prognostic, therapeutic, and research purposes.

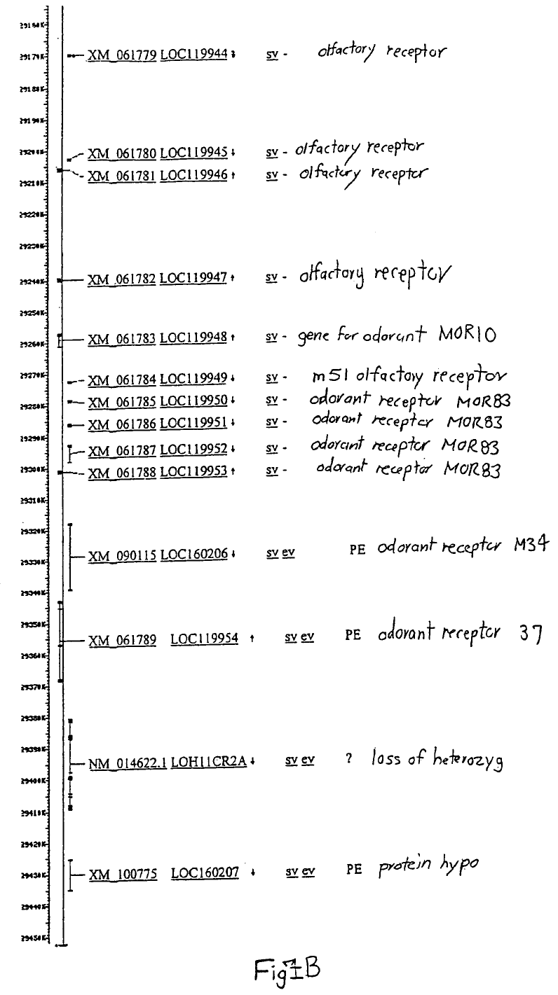

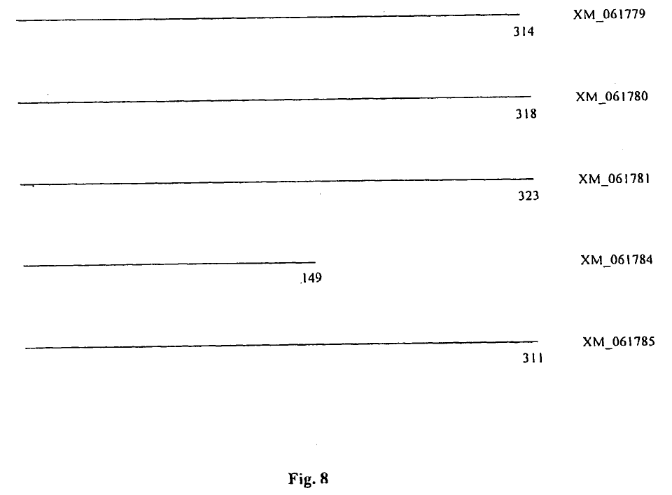

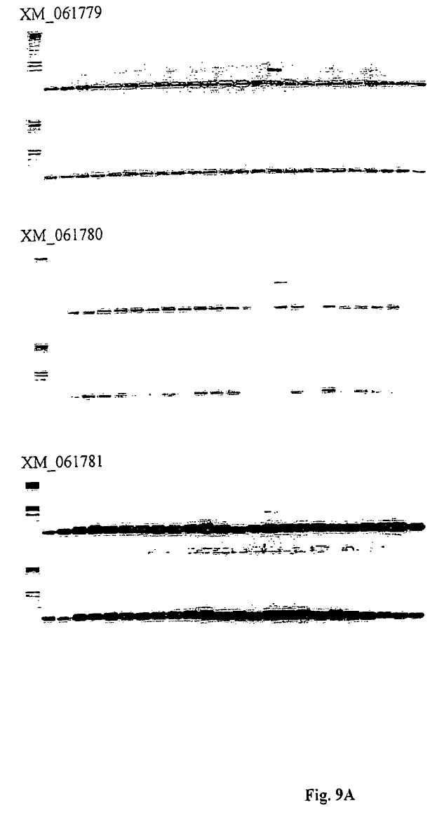

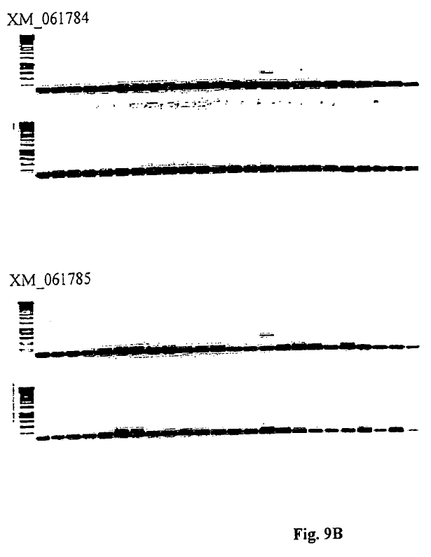

The spatial organization of the pancreatic gene complex (“TGC”) is illustrated in FIG. 7. It spans several hundred kilobases of chromosome 11, e.g., from about LOC160205 to LOC119954, from about LOC119944-LOC119954, and any part thereof. Within this region, is a cluster of genes coding for polypeptides which share sequence identity with the olfactory GPCR family. These include, but are not limited to, TMD0986, XM_{—}061780 (TMD0987), XM_{—}061781 (TMD0353), XM_{—}061784 (TMD0989), XM_{—}061785 (TMD058). FIG. 8 illustrates the relationship between the lengths of the different coding sequences. As shown in the figure, XM_{—}061784 is shorter at its C-terminus than the other family members.

As members of the GPCR family, the PGC genes all share a degree of amino acid sequence identity and similarity. See, Table 14 for values (% sequence identity is the first place; % sequence similarity is in parenthesis in the second place; calculations were performed using the publicly-available BLASTP pair-wise alignment program). TMD0986, XM_{—}061780, XM_{—}061781, and XM_{—}061785 each share about 40% sequence identity. BLAST searching of publicly available sequences indicates that these polypeptides share less amino acid sequence identity with each other than they do with other olfactory GPCR homologs located elsewhere in the genome. Significantly higher amino acid sequence identity—81%—is observed between the adjacent genes XM_{—}061784 and XM_{—}061785. These genes appear to be part of a sub-cluster within PGC that share high polypeptide similarity between them.

The phrase “a gene of Table 12“which is used throughout the description include the specific sequences for the listed XM numbers as well as other human alleles, and mammalian homologs, such as murine homologs. For example, Table 14 lists several of the mouse homologs that are included in the present invention. While SEQ ID NOS. 152, 153, 162, 163, 167, 168, 171, 172, 175, and 176 may represent particular alleles, the present invention relates to other alleles, as well, including naturally-occurring polymorphisms (i.e., a polymorphism in a nucleotide sequence which is identified in populations of mammals).

TMD0986 (SEQ ID NO 152 and 153) is a full-length sequence of the previously identified XM_{—}061779. It contains an additional 117 amino acids not present in XM_{—}061779. The present invention relates to nucleic acids comprising or consisting essentially of this sequence in its entirety (e.g., amino acids 1-314), comprising or consisting essentially of nucleic acids coding for amino acids 1-117, and comprising or consisting essentially of fragments of nucleic acids coding for amino acids 1-117. Polypeptides encoded by these nucleic acids are also claimed, including polypeptide fragments of 1-117, such as 1-23, 79-97, 164-198, 261-274, and other extracellularly exposed peptides. In addition, the present invention relates to binding partners, such as antibodies, that bind to epitopes within amino acids 1-117 (e.g., SEQ ID NO 153).

Pancreas

Diabetes and other pancreatic disorders are a major health concern. Worldwide, it is estimated that 5-10% of the population suffers from some form of diabetes. Pancreatic cancer is the fifth leading cause of cancer-related mortality. In 2002, it was estimated that about 30,000 Americans would be diagnosed with pancreatic cancer, and 90% would die within 12 months. Despite the prevalence of pancreatic disease, the genetics and physiology of normal pancreatic function and pancreatic disease is still poorly understood.

The pancreas is a mixed gland comprised of exocrine and endocrine tissues. The exocrine portion comprises about 80-85% of the organ. It is divided into lobes by connective tissue septa, and each lobe is divided into several lobules. These lobules are composed of grape-like clusters of secretory cells that form sacs known as acini. An acinus is a functional unit of the pancreatic exocrine gland. All acini drain into interlobular ducts which merge to form the main pancreatic duct. It, in turn, joins together with the bile duct from the liver to form the common bile duct that empties into the duodenum. Pancreatic acinar cells make up more than 80% of the total volume of the pancreas and function in the secretion of the various enzymes that assist digestion in the gastrointestinal tract. Scattered among the acinar cells are approximately a million pancreatic islets (“islets of Langerhans”) that secrete the pancreatic endocrine hormones. These dispersed islets comprise approximately 2% of the total volume of the pancreas.

The basic function of the pancreatic endocrine cells is to secrete certain hormones that participate in the metabolism of proteins, carbohydrates, and fats. The hormones secreted by the islets include, e.g., insulin, glucagon, somatostatin, pancreatic polypeptide, amylin, adrenomedullin, gastrin, secretin, and peptide-YY. See, also, Shimizu et al., Endocrin., 139:389-396, 1998. The islets contain about four major and two minor cell types. The major cell types are alpha (glucagon producing), beta (insulin and amylin producing), delta (somatostatin producing which suppresses both insulin and glucagon release), and F (pancreatic polypeptide and adrenomedullin producing) cells. The minor cell types are D1 (produce vasoactive intestinal peptide or VIP) and enterochromaffin (produce serotonin) cells. The cells can be distinguished, e.g., by their morphology, hormonal content, and polynucleotide expression patterns.

The ability of the pancreas to respond to a wide variety of metabolic signals is conferred by an expression profile comprising a rich assortment of receptor proteins. G-protein coupled receptors have been previously identified in the pancreas, including, e.g., receptors for glucagon, secretin, CCK (e.g., Roettger et al., J. Cell Biol., 130:579-590, 1995), purines (e.g., P2 purinoreceptors), gastrin, KiSS-1 peptides (e.g., Kotani et al., J. Biol. Chem., 276:34631-6, 2001), adrenomedullin (Martinez et al., Endocrin., 141:406, 2000), and interleukins. G-protein subunits have also been localized to the pancreas, including G-proteins which were previously associated with the olfactory epithelium. See, e.g., Zigman et al., Endocrin., 133:2508-2514, 1993. In addition, pancreatic cells express neurotropin, neurotensin, and interleukin receptors.

As mentioned, the pancreas is sensitive to a variety of metabolic, soluble and hormonal signals involved in regulating blood sugar, modulating synthesis and release of pancreatic digestive enzymes, and other physiologically important processes involved in pancreas function. In analogy to the ability of olfactory receptors to detect odors and pheromones in the environment, the pancreatic GPCRs of the present invention can be used to “sniff” out and respond to various ligands in the blood which pass through the pancreas, including peptides, metabolites, and other biologically-active molecules. Biological activities include, but are not limited to, e.g., regulation of blood sugar, modulation of all aspects of the various secreted polypeptides (hormones, enzymes, etc.) produced by the pancreas, ligand-binding, exocytosis, amylase (and any of the other 20 or so digestive enzymes produced by the pancreas) secretion, autocrine responses, apoptosis (e.g., in the survival of beta-islet cells), zymogen granule processing, G-protein coupling activity, etc.

The polynucleotides, polypeptides, and ligands thereto, of the present invention can be used to identify, detect, stage, determine the presence of, prognosticate, treat, study, etc., diseases and conditions of pancreas. These include, but are not limited to, e.g., disorders associated with loss or mutation to 11q24, such as Jacobsen syndrome (OMIN #147791), cystic fibrosis, acute and chronic pancreatitis, pancreatic abscess, pancreatic pseudocyst, nonalcoholic pancreatitis, alcoholic pancreatitis, classic acute hemorrhagic pancreatitis, chronic calcifying pancreatitis, familial hereditary pancreatitis, carcinomas of the pancreas, primary (idiopathic) diabetes (e.g., Type I (insulin dependent diabetes mellitus, IDDK) [insulin deficiency, beta cell depletion], Type II (non-insulin dependent diabetes mellitus, NIDDM) [insulin resistance, relative insulin deficiency, mild beta cell depletion]), nonobese NIDDM, obese NMDDM, maturity-onset diabetes of the young (MODY), islet cell tumors, diffuse hyperplasia of the islets of Langerhans, benign adenomas, malignant islet tumors, hyperfunction of the islets of Langerhans, hyperinsulinism and hypoglycemia, Zollinger-Ellison syndrome, beta cell tumors (insulinoma), alpha cell tumors (glucagonoma), delta cell tumors (somatostatinoma), vipoma (diarrheogenic islet cell tumor), pancreatic cancers, pancreatic carcinoid tumors, multihormonal tumors, multiple endocrine neoplasia (MEN), MEN I (Wermer syndrome), MEN II (Sipple syndrome), MEN III or IIb, pancreatic endocrine tumors, etc.

In view of its selectivity and display on the cell surface, the olfactory GPCR family members of the present invention are useful targets for histological, diagnostic, and therapeutic applications relating to the cells (e.g., pancreatic progenitor, exocrine, endocrine, acinar, islet, alpha, beta, delta, F, D1, enterochromaffin, etc.) in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. They can also be used to detect metastatic cells, in biopsies to identify bone marrow, lymphocytes, etc. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly as indicated in Table 14. Any of the methods described above and below can be accomplished in vivo, in vitro, or ex vivo.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

The present invention relates to methods of detecting pancreas cells, comprising one or more of the following steps, e.g., contacting a sample comprising cells with a polynucleotide specific for a gene of Table 12, or a mammalian homolog thereof under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID NOS 154, 155, 164, 165, 169, 170, 173, 174, 177, and 178, and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting a pancreas cell, comprising, one or more the following steps, e.g. contacting a sample comprising cells with a binding partner (e.g. an antibody, an Fab fragment, a single-chain antibody, an aptamer) specific for a polypeptide coded for by a polypeptide of Table 12, or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface as indicated in Table 14.

As indicated above, binding partners can be used to deliver agents specifically to the pancreas, e.g., for diagnostic, therapeutic, and prognostic purposes. Methods of delivering an agent to a pancreas cell can comprise, e.g., contacting a pancreas cell with an agent coupled to a binding partner specific for a polypeptide coding for a gene of Table 12, whereby said agent is delivered to said cell. Any type of agent can be used, including, therapeutic and imaging agents. Contact with the pancreas can be achieved in any effective manner, including by administering effective amounts of the agent to a host orally, parentally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers. Any cell expressing a polypeptide coded for by a gene of Table 12 can be targeted, including, e.g., pancreatic progenitor, exocrine, endocrine, secretory, acinar, islet, alpha, beta, delta, F, D1, enterochromaffin, etc.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos, 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose. See, Bruehlmeier et al., Nucl. Med. Biol., 29:321-327, 2002, for imaging pancreas using labeled receptor ligands. Antibodies and other ligands to receptors of the present invention can be used analogously.

A pancreas cell (see above for examples of pancreas cell types) can also be modulated in accordance with the present invention, e.g., by methods of modulating a pancreas cell, comprising, e.g., contacting said cell with an agent effective to modulate a gene of Table 12, or the biological activity of a polypeptide encoded thereby (e.g., SEQ ID NO 153, 163, 168, 172, or 176), or a mammalian-homolog thereof, whereby said pancreas cell is modulated. Modulation as used throughout includes, e.g., stimulating, increasing, agonizing, activating, amplifying, blocking, inhibiting, reducing, antagonizing, preventing, decreasing, diminishing, etc.

An activity or function of the pancreas cell can be modulated, including, e.g., regulation of blood sugar, modulation of all aspects of the various secreted polypeptides (hormones, enzymes, etc.) produced by the pancreas, ligand-binding, exocytosis, amylase (and any of the other 20 or so digestive enzymes produced by the pancreas) secretion, autocrine responses, apoptosis (e.g., in the survival of beta-islet cells), etc.

The present invention also relates to polypeptide detection methods for assessing pancreas function, e.g., methods of assessing pancreas function, comprising, detecting a polypeptide coded for by a gene of Table 12, fragments thereof, polymorphisms thereof, in a body fluid, whereby the level of said polypeptide in said fluid is a measure of pancreas function. Pancreas function tests are usually performed to determine whether the pancreas is functioning normally as a way of diagnosing pancreas disease. Various tests are commonly used, including, e.g., assays for the presence of pancreatic enzymes in body fluids (e.g., amylase, serum lipase, serum trypsin-like immuoreactivity), studies of pancreatic structure (e.g., using x-ray, sonography, CT-scan, angiography, endoscopic retrograde cholangiopancreatography), and tests for pancreatic function (e.g., secretin-pancreozymin (CCK) tst, Lundh meal test, Bz-Ty-PABA test, chymotrypsin in feces, etc). Detection of a polypeptide coded for by a gene of Table 12 provides an additional assessment tool, especially in diseases such as pancreatitis and pancreatic cancer where pancreatic markers can appear in the blood, stool, urine, and other body fluids. As with the other tests, elevated levels of said polypeptide in blood, or other fluids, can indicate impaired pancreas function. Values can be determined routinely, as they are for other markers , such as those mentioned above. Detecting can be performed routinely (see below), e.g., using an antibody which is specific for said polypeptide, by RIA, ELISA, or Western blot, etc., in analogy to the tests for pancreatic enzymes in body fluids.

Promoter sequences obtained from GPCR genes of the present invention can be utilized to selectively express heterologgus genes in pancreas cells. Methods of expressing a heterologous polynucleotide in pancreas cells can comprise, e.g., expressing a nucleic acid construct in pancreas cells, said construct comprising a promoter sequence operably linked to said heterologous polynucleotide, wherein said promoter sequence is selected SEQ ID NOS 156-161, 166, 179, or 180. In addition to the cell lines mentioned below, the construct can be expressed in primary cells or in established cell lines.

The genes and polypeptides of Table 12 can be used to identify, detect, stage, determine the presence of, prognosticate, treat, study, etc., diseases and conditions of the pancreas as mentioned above. The present invention relates to methods of identifying a pancreatic disease or pancreatic disease-susceptibility, comprising, e.g., determining the association of a pancreatic disease or pancreatic disease-susceptibility with a nucleotide sequence present within the pancreatic gene complex. An association between a pancreas disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target.

Any region of the pancreatic gene complex can be used as a source of the DNA marker (e.g., a nucleotide sequence present with PGC), including, e.g., TMD0986, XM_{—}061780 (TMD0987), XM_{—}061781 (TMD0353), XM_{—}061784 (TMD0989), XM_{—}061785 (TMD058), and any part thereof, introns, intergenic regions, any DNA from about 29160-29310 kb of 11q24, NT_{—}009215, etc.

Human linkage maps can be constructed to establish a relationship between a region within 11q24 and a pancreatic disease or condition. Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype.

Retina Selective Gene

The present invention relates to NM_{—}013941 (GPCR181 or OR10Cl), a multiple transmnembrane spanning polypeptide which shares sequence identity, with the olfactory G-protein coupled receptor (GPCR) family. Like other GPCR, NM_{—}013941 has seven transmembrane domains, at about amino acid positions 20-42, 54-76, 91-113, 134-156, 190-212, 233-255, and 265-287, of SEQ ID NO 182. It is located at about chromosomal band 6p21.31-22.2. There are several other GPCRs located nearby (e.g., OR2B3, AL022727; OR2J3, AL022727). NM_{—}013941 is highly expressed in brain tissue, at lower levels in heart, pituitary, and skin, and at minimally detectable levels in colon, small intestine, kidney, lymphocytes, and mammary gland. In the neuronal tissue, it was selectively expressed in the retina, but was not detected in any other brain tissue regions. The selective expression of NM_{—}013941 in the retina makes it useful as a marker for retinal tissue, e.g., in stem cell cultures and biopsy samples, as well as a diagnostic, prognostic, therapeutic, and research tool for any conditions, diseases, disorders, or applications associated with the retina and the cells in which it is expressed. NM_{—}013941 includes both human and mammalian homologs of it (e.g., mouse XM_{—}111729 which is similar to olfactory receptor MOR263-6). SEQ ID NOS. 181 and 182 represent a particular allele of NM_{—}013941; the present invention relates to other alleles, as well, including naturally-occurring polymorphisms (i.e., a polymorphism in the nucleotide sequence which is identified in populations of mammals).

The chromosomal region within which NM_{—}013941 is located comprises a number of genes involved in retinal function. These include, e.g., retinal cone dystrophy (OMIM 602093) which appears to be a result of mutation in guanylate cyclase activator-1A (e.g., Payne et al., Human Molec. Genet., 7:273-277, 1998), retinal degeneration slow (OMIM 179605) which appears to be a defect in specific retinal protein homologous to rod outer segment protein-1, retinitis pigmentosa-7, retinitis pigrnentosa-14 (OMIM 600132) which is associated with a mutation in the tubby-like protein TULP1 (e.g., Banctee et al., Nature Genet., 18:177-179, 1998; Hagstrom et al., Nature Genet., 18:174-176, 1998), and others. Thus, this region appears to be important in eye function.

In view of its selectivity and display on the cell surface, the olfactory GPCR family members of the present invention are useful targets for histological, diagnostic, and therapeutic applications relating to retinal cells. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat retinal carcinomas (e.g., retinoblastoma) in analogy to how c-erbB-2 antibodies are used to breast cancer. See, e.g., Hayashi et al., Invest. Ophthalmol. Vis. Sci., 40:265-72, 1999 for an example treating retinoblastoma using HSV-TK. Transfer of the gene into the retinal cells can be achieved by incorporating the gene into liposomes which have been made cell-selective by incorporating a NM_{—}013941 specific antibody into-its bilayer. See, also, Wu and Wu, J. Biol. Chem., 262: 44294432, 1987.

The genes and polypeptides encoded thereby can also be used in tissue engineering to identify tissues as they appear during the differentiation process, to target tissues, to modulate tissue growth (e.g., from starting stern cell populations), etc. Useful antibodies or other binding partners include those that are specific for parts of the polypeptide which are exposed extracellularly. Any of the methods described above and below can be accomplished in vivo, in vitro, or ex vivo.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

The present invention relates to methods of detecting retinal cells, comprising one or more of the following steps, e.g., contacting a sample comprising cells with a polynucleotide specific for NM_{—}013941 (e.g., SEQ ID NOS 181), or a mammalian homolog thereof, under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID NOS 183 and 184, and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting a retinal cell, comprising, one or more the following steps, e.g. contacting a sample comprising cells with a binding partner (e.g. an antibody, an Fab fragment, a single-chain antibody, an aptamer) specific for a polypeptide coded for by NM_{—}013941 (e.g., SEQ ID NO 182), or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface.

As indicated above, binding partners can be used to deliver agents specifically to the retina, e.g., for diagnostic, therapeutic, and prognostic purposes. Methods of delivering an agent to a retinal cell can comprise, e.g., contacting a retinal cell with an agent coupled to binding partner specific for NM_{—}013941 (SEQ ID NO 182), whereby said agent is delivered to said cell. Any type of agent can be used, including, therapeutic and imaging agents. Contact with the retinal can be achieved in any effective manner, including by administering effective amounts of the agent to a host orally, parentally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers. Any cell expressing a polypeptide coded for by NM_{—}013941 can be targeted, including, e.g., pigmented epithelial cells, photoreceptor cells, cones, rods, bipolar cells, ganglion cells, etc.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos, 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706and 6,221,334., The methods described therein can be used generally to associate a partner with an agent for any desired purpose.

A retinal cell (see above for examples of retinal cell types) can also be modulated in accordance with the present invention, e.g., by methods of modulating a retinal cell, comprising, e.g., contacting said cell with an agent effective to modulate NM_{—}013941, or the biological activity of a polypeptide encoded thereby (e.g., SEQ ID NO 182), or a mammalian homolog thereof, whereby said retinal cell is modulated. Modulation as used throughout includes, e.g., stimulating, increasing, agonizing, activating, amplifying, blocking, inhibiting, reducing, antagonizing, preventing, decreasing, diminishing, etc.

Any activity or function of the retinal cell can be modulated, including, e.g., light reception, phototransduction, excitation of rods, excitation of cones, metabolism of vitamin A, retinal, rhodopsin, and other functional molecules, cGMP binding and hydrolysis, sodium channel flux, membrane potential, phosphodiesterase activity, G-protein activity and coupling, vitamin A processing, sodium pump activity, calcium flux, etc. The response of a retinal cell to stimuli can also be modulated, including, but not limited to, ligands to NM_{—}013941, light, ion levels, second messenger levels, etc.

Promoter sequences can be utilized to selectively express heterologous genes in retinal cells. Methods of expressing a heterologous polynucleotide in retinal cells can comprise, e.g., expressing a nucleic acid construct in retinal cells, said construct comprising a promoter sequence operably linked to said heterologous polynucleotide, wherein said promoter sequence is obtained from NM_{—}01394, e.g., on genomic NT_{—}007592. In addition to the cell lines mentioned below, the construct can be expressed in primary cells or in established cell lines.

Retina

The retina is a two-layered structure located on the back of the eye. It is the primary organ responsible for vision. The outer pigmented layer is comprised of pigmented epithelial cells that absorb light, preventing it from scattering in the eye, and store vitamin A needed by the photoreceptor cells. The inner neural layer is comprised of three main cell types: photoreceptor cells, bipolar cells, and ganglion cells. The local currents generated by a light stimulus-spreads from the photoreceptor cells to the bipolar cells, and then on to the innermost ganglion-cells. The optic disc-is the exit site of the retinal ganglion axons which then bundle into the optic nerve

Photoreceptors consist of rods and cones which are the photosensitive cells of the retina. Each rod and cone elaborates a specialized cilium, called the outer segment, that contains the phototransduction machinery. The rods contain a specific light-absorbing visual pigment, rhodopsin. In humans, there are three classes of cones, each characterized by the expression of distinct visual pigments: the blue cone, green cone and red cone pigments. Each type of visual pigment protein is tuned to absorb light maximally at different wavelengths. The rod rhodopsin mediates scotopic vision (in dim light), whereas the cone pigments are responsible for photopic vision (in bright light). The red, blue and green pigments also form the basis of color vision.

NM_{—}013941 can be used to identify, detect, stage, determine the presence of, prognosticate, treat, study, etc., diseases and conditions of the retinal. These include, but are not limited to, diseases that affect the basic morphologic components as mentioned above, e.g., the outer and inner cell layers, and the optic nerve the retina. Diseases include, e.g., retinal degeneration, retinal degenerations such as retinitis pigmentosa, Bardet-Biedi syndrome, Bassen-Kornzweig syndrome (abetalipoproteinemia), Best disease (vitelliforrn dystrophy), choroidemia, gyrate atrophy, congenital amaurosis, Refsum syndrome, Stargardt disease, Usher syndrome, macular degeneration (dry and wet forms), diabetic retinopathy, peripheral vitreoretinopathies, photic retinopathies, surgery-induced retinopathies, viral retinopathies (such as HIV retinopathy related to AIDS), ischemnic retifopathies, retinal detachment, traumatic retinopathy, optic neuropathy, optic neuritis, ischemic optic neuropathy, Leber optic neuropathy, diseases of Bruch's membrane, glaucoma, cancer, retinoblastoma, cancer-associated retinopathy syndrome (CAR syndrome), melanoma-associated retinopathy (MAR), etc. NM_{—}013941 can also be used for staging and classifying conditions and diseases of the present invention, alone, or in combination with conventional staging and classification schemes.

Spleen Gene Cluster

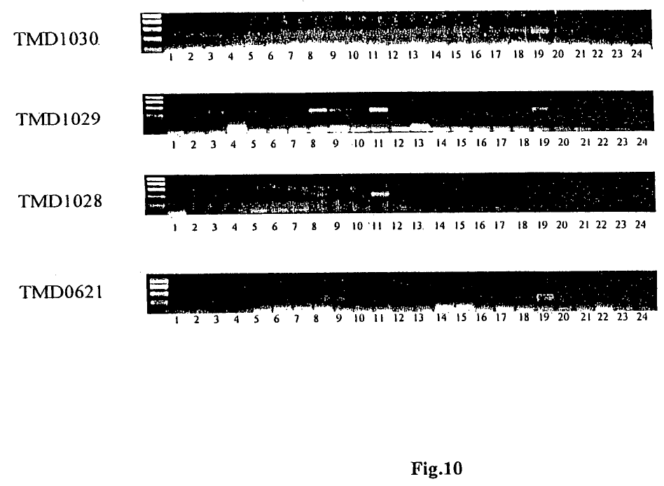

The present invention relates to a cluster of transmnembrane and GPCR-type receptor genes located at chromosomal band 11q12.2. The genes of the present invention are expressed predominantly in the spleen (e.g., FIG. 10, lane 19) (hence, “spleen gene” cluster), as well as other tissues of the immune and reticuloendothelial system (RES), establishing this region of the chromosome as a unique gene complex involved in spleen, lymphoid, and/or reticuloendothelial function. TMD1030 and TMD0621 are highly expressed in spleen tissue, with insignificant levels in other tissues. In addition to spleen. TMD1029 and TMD1029 show significant expression in the liver and lymphocytes, as well. Because of their selectivity for spleen, lymphoid, and/or reticuloendothelial tissues, the gene complex, and the chromosomal region which comprises it, are useful to assess spleen, lymphoid, and/or reticuloendothelial tissue function and for diagnostic, prognostic, therapeutic, and research purposes. Information on the genes is summarized in Tables 15-19.

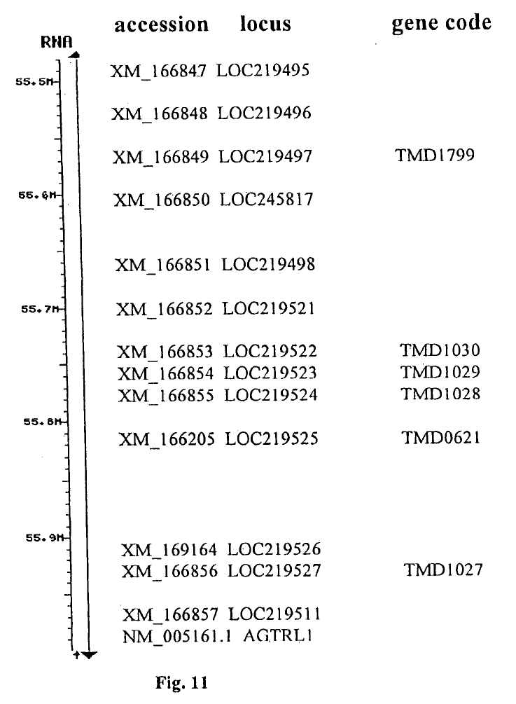

The spatial organization of the gene complex is illustrated in FIG. 11. The complex spans about at least 100 kb, from about EST markers G62658, SHGC-82134, etc. (located at the end closest to the centromere and TMD1030) to SHGC-154002, SHGC-9433, etc. (located at the end first from the centromere and TMD0621). All the genes have the same orientation of transcription. TMD 1799 (MM_{—}166849) (SEQ ID NO 193-194), located at the upper region, shows very high expression in lymphocytes, but only marginal expression in spleen, indicating that expression in lymphocytes may predominate at the boundaries of the gene complex. In the lower region, TMD1027 (XM_{—}66856) (SEQ ID NO 195-196), spleen expression virtually disappears, while lymph node expression becomes very high. The present invention includes this entire region, and any parts thereof. For instance, the present invention includes any DNA fragments within it which confer the observed tissue specificities described herein.

The gene complex is involved in spleen, immune, and RES functions. The spleen is located in the left upper region of the abdomen. In the adult, it weights about 90-180 grams, and is about 15 by 7.5 cm in size. The spleen is anatomically and functionally compartmentalized into two distinct regions, the red and white pulp. The red pulp comprises blood vessels interwoven with connective tissue (“pulp cords”) that is lined with reticuloendothelial cells. It possesses a blood filtering function, removing opsonized cells and trapping abnormal red blood cells. It also is a storage reservoir for platelets and other blood cells. In the fetus, the red pulp has a hematopoietic function. Inside the red pulp, is lymphoid tissue know as the white pulp. Antibodies are made inside the white pulp. Similar to other lymphatic tissues, B- and T-cell's mature inside the white pulp, where they are involved in antigen presentation and lymphocyte maturation. The white pulp is clustered around the periarteriolar lymphoid sheath, and is comprised of follicles and marginal zone. Naive B-cells are located in the primary follicle, memory cells, macrophages, and dendritic cells in the secondary follicle, and macrophages and B-cells in the marginal zone. The integrins LFA-1 and alpha4-betal are involved in localization of the B-cells to the marginal zone of the white pulp (Lu and Cyster, Science, 297:409, 2002).

The reticuloendothelial system (RES) is a multi-organ phagocytic system involved in removing particulates from the blood. It is comprised of the spleen and liver. It has the ability to sequester inert particles and dyes. Cells of the RES system include, macrophages, liver Kuppfer cells, endothelial cells lining the sinusoids of the liver, spleen, and bone marrow, and reticular cells of lymphatic and bone marrow tissues.

The polynucleotides, polypeptides, and ligands thereto, of the present invention can be used to identify, detect, stage, determine the presence of, prognosticate, treat, study, etc., diseases and conditions of spleen, lymphoid, and/or reticuloendothelial tissues. These include, but are not limited to, splenomegaly, hypersplenism, hemolytic anemis, hereditary spherocytosis, hereditary eliptocytosis, thalassemia minor and major, autoimmune hemolytic anemia, thrombocytopenia, idiopathic thrombocytopenic purpura, immunologic thrombocytopenia associated with chronic lymphocytic leukenaia or systemic lupus erythematosis, TTP, leukemia, lymphoma, primary and metastatic tumors, splenic cysts, infection, inflammatory diseases, anemias, blood cancers, etc. See, Table 19 for other examples.

In view of their selectivity and display on the cell surface, the genes of the present invention are useful targets for histological, diagnostic, and therapeutic applications relating to the cells (e.g., reticuloendothelial cells, macrophages, Kupffer cells, monocytes, B-lymphocytes, T-lymphocytes, etc) in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to treat breast cancer. They can also be used to detect metastatic cells in biopsies. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly. See, Table 16. Any of the methods described above and below can be accomplished in vivo, in vitro, or ex vivo.

When expression is described as being “predominantly” in a given tissue, this indicates that the gene's mRNAs levels are highest in this tissue as compared to the other tissues in which it was measured. Expression can also be “selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types. TMD1030 and TMD0621 are predominantly and selectively expressed in spleen tissue.

The expression patterns of the selectively expressed polynucleotides disclosed herein can be described as a “fingerprint” in that they are a distinctive pattern displayed by a tissue. Just as with a fingerprint, an expression pattern can be used as a unique identifier to characterize the status of a tissue sample. The list of expressed sequences disclosed herein provides an example of such a tissue expression profile. It can be used as a point of reference to compare and characterize samples. Tissue fingerprints can be used in many ways, e.g., to classify an unknown tissue, to determine the origin of metastatic cells, to assess the physiological status of a tissue, to determine the effect of a particular treatment regime on a tissue, to evaluate the toxicity of a compound on a tissue of interest, etc.

For example, the tissue-selective polynucleotides disclosed herein represent the configuration of genes expressed by a normal tissue. To determine the effect of a toxin on a tissue, a sample of tissue can be obtained prior to toxin exposure (“control”) and then at one or more time points after toxin exposure (“experimental”). An array of tissue-selective probes can be used to assess the expression patterns for both the control and experimental samples. As discussed in more detail below, any suitable method can be used. For instance, a DNA microarray can be prepared having a set of tissue-selective genes arranged on to a small surface area in fixed and addressable positions. RNA isolated from samples can be labeled using reverse transcriptase and radioactive nucleotides, hybridized to the array, and then expression levels determined using a detection system. Several kinds of information can be extracted: presence or absence of expression, and the corresponding expression levels. The normal tissue would be expected to express substantially all the genes represented by the tissue-selective probes. The various experimental conditions can be compared to it to determine whether a gene is expressed, and how its levels match up to the normal control.

While the expression profile of the complete gene set represented by the sequences disclosed here may be most informative, a fingerprint containing expression information from less than the full collection can be useful, as well. In the same way that an incomplete fingerprint may contain enough of the pattern of whorls, arches, loops, and ridges, to identify the individual, a cell expression fingerprint containing less than the fill complement may be adequate to provide useful and unique identifying and other information about the sample. Moreover, because of heterogeneity of the population, as well differences in the particular physiological state of the tissue, a tissue's “normal” expression profile is expected to differ between samples, albeit in ways that do not change the overall expression pattern. As a result of these individual differences, each gene although expressed selectively in spleen, may not on its own 100% of the time be adequately enough expressed to distinguish said tissue. Thus, the genes can be used in any of the methods and processes mentioned above and below as a group, or one at a time.

The present invention relates to methods of detecting spleen, lymphoid, and/or reticuloendothelial cells, comprising one or more of the following steps, e.g., contacting a sample comprising cells with a polynucleotide specific for TMD1030 (XM_{—}166853), TMD1029 (XM_{—}166854), TMD1028 (XM_{—}166855), or TMD0621 (XM_{—}166205), or a mammalian homolog thereof, under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include SEQ ID NOS 197-204 listed in Table 17, and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting a spleen, lymphoid, and/or reticuloendothelial cell, comprising, one or more the following steps, e.g. contacting a sample comprising cells with a binding partner (e.g. an antibody, an Fab fragment, a single-chain antibody, an aptamer) specific for a polypeptide coded for by a polypeptide of the present invention, or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface. Detection can be useful for assessing spleen integrity, e.g., when it is suspected that the spleen is damaged and undergoing deterioration. The appearance of polypeptides of the present invention in body fluids, such as blood, can indicate spleen damage, including neoplastic and/or apoptotic changes.

As indicated above, binding partners can be used to deliver agents specifically to the spleen, lymphoid, and/or reticuloendothelial tissues, e.g., for diagnostic, therapeutic, and prognostic purposes. Methods of delivering an agent to a spleen, lymphoid, and/or reticuloendothelial cell can comprise, e.g., contacting a spleen, lymphoid, and/or reticuloendothelial cell with an agent coupled to a binding partner specific for a polypeptide coding for TMD1030 (XM_{—}166853), TMD1029 (XM_{—}166854), TMD1028 (XM_{—}166855), or TMD0621 (XM_{—}166205), whereby said agent is delivered to said cell. Any type of agent can be used, including, therapeutic and imaging agents. Contact with the spleen, lymphoid, and/or reticuloendothelial tissue can be achieved in any effective manner, including by administering effective amounts of the agent to a host orally, parenterally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers. Any cell expressing a polypeptide coded for by TMD1030 (XM_{—}166853), TMD1029 (XM_{—}166854), TMD1028 (XM_{—}166855), or TMD0621 (XM_{—}166205) can be targeted, including, e.g., reticuloendothelial cells, macrophages, Kupffer cells, lymphocytes, B-lymphocytes, T-lymphocytes, etc.

Antibodies (alone or conjugated to active agents) can be used to ablate spleen and other tissues. For instance, in diseases where splenectomy is indicated (e.g., immune thrombocytopenic purpura, autoimmune hemolytic anemia, blood cell disorders, myeloproliferative disorders, tumors, hypersplenism, etc.), antibodies to TMD1030 and TMD0621 can be used to ablate spleen tissue, or block spleen function.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintiographic imaging. A reporter agent can be conjugated or associated routinely with a binding partner. Ultrasound contrast agents combined with binding partners, such as antibodies, are described in, e.g., U.S. Pat. Nos, 6,264,917, 6,254,852, 6,245,318, and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose. See, Bruehlmeier et al., Nucl. Med. Biol., 29:321-327, 2002, for imaging using labeled receptor ligands. Antibodies and other ligands to receptors of the present invention can be used analogously.

A cell (see above for examples of spleen, lymphoid, and/or reticuloendothelial cell types) can also be modulated in accordance with the present invention, e.g., by methods of modulating a spleen, lymphoid, and/or reticuloendothelial cell, comprising, e.g., contacting said cell with an agent effective to modulate TMD1030 (XM_{—}66853), TMD1029 (XM_{—}66854), TMD1028 (XM_{—}166855), or TMD0621 (XM_{—}166205), or the biological activity of a polypeptide encoded thereby (e.g., SEQ ID NOS 185-192), or a mammalian homolog thereof, whereby said spleen, lymphoid, and/or reticuloendothelial cell is modulated. Modulation as used throughout includes, e.g., stimulating, increasing, agonizing, activating, amplifying, blocking, inhibiting, reducing, antagonizing, preventing, decreasing, diminishing, etc.

Any activity or function of the spleen, lymphoid, and/or reticuloendothelial tissues can be modulated, including, e.g., immune modulation (e.g., modulating antigen presentation, antibody production and secretion, humoral and cellular responses, etc.), sequestration and removal of red blood cells, clearance of microorganisms and particular antigens from blood, migration into the marginal zone or other immune and RES compartments, etc. The present invention also relates to polypeptide detection methods for assessing spleen, lymphoid, and/or reticuloendothelial tissue function, e.g., methods of assessing spleen, lymphoid, and/or reticuloendothelial function, comprising, detecting a polypeptide coded for by TMD1030 (XM_{—}166853), TMD_{—}1029 (XM_{—}166854), TMD1028 (XM_{—}166855), or TMD0621 (XM_{—}66205), fragments thereof, polymorphisms thereof, in a body fluid, whereby the level of said polypeptide in said fluid is a measure of spleen, lymphoid, and/or reticuloendothelial function, spleen, lymphoid, and/or reticuloendothelial function tests are usually performed to determine whether the spleen, lymphoid, and/or reticuloendothelial tissue is functioning normally as a way of diagnosing spleen, lymphoid, and/or reticuloendothelial disease. Various tests are commonly used, including, e.g., 99Tc-colloid liver-spleen scan, computed tomography, ultrasound scanning of left upper quandrant, MRI, liver enzymes, etc.

Detection of a polypeptide coded for by TMD1030 (XM_{—}166853), TMD1029 (XM_{—}166854), TMD1028 (XM_{—}166855), or TMD0621 (XM_{—}166205), provides an additional assessment tool, especially in diseases or disorders, such as splenomegaly, hypersplenism, or ruptured spleen, where said polypeptides can appear in the blood, stool, urine, and other body fluids. As with the other tests, elevated levels of said polypeptide in blood, or other fluids, can indicate impaired spleen, lymphoid, and/or reticuloendothelial function. Values can be determined routinely, as they are for other markers, such as those mentioned above. Detecting can be performed routinely (see below), e.g., using an antibody which is specific for said polypeptide, by RIA, ELISA, or Western blot, etc., in analogy to the tests for enzymes and other proteins in body fluids.

Promoter sequences obtained from genes of the present invention can be utilized to selectively express heterologous genes in cells. Methods of expressing a heterologous polynucleotide in cells, e.g., spleen, lymphoid, and/or reticuloendothelial cells can comprise, e.g., expressing a nucleic acid construct in spleen, lymphoid, and/or reticuloendothelial cells, said construct comprising a promoter sequence operably linked to said heterologous polynucleotide, wherein said promoter sequence is selected SEQ ID NOS 205-213. In addition to the cell lines mentioned below, the construct can be expressed in primary cells or in established cell lines.

The genes and polypeptides of the present invention can be used to identify, detect, stage, determine the presence of, prognosticate, treat, study, etc., diseases and conditions of the spleen, lymphoid, and/or reticuloendothelial tissues mentioned above. The present invention relates to methods of identifying a genetic basis for a disease or disease-susceptibility, comprising, e.g., determining the association of a spleen, lymphoid, and/or reticuloendothelial disease or spleen, lymphoid, and/or reticuloendothelial disease-susceptibility with the gene complex of the present invention, e.g., a nucleotide sequence present in the gene complex at 11q12.2. An association between a spleen, lymphoid, and/or reticuloendothelial disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target

Any region of the gene can be used as a source of the DNA marker, exons, introns, intergenic regions, or any DNA from the gene cluster of the present invention at chromosomal region 11q12.2, etc.

Human linkage maps can be constructed to establish a relationship between a gene and a spleen, lymphoid, and/or reticuloendothelial disease or condition. Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced for an individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype.

The present invention also relates to methods of expressing a polynucleotide in spleen, lymphoid, and/or reticuloendothelial tissue, comprising, e.g., inserting a polynucleotide, which is operably linked to an expression control sequence, into the spleen, lymphoid, and/or reticuloendothelial gene complex at chromosomal location 11q12.2 of a target cell, and growing said cell under conditions effective to express said polynucleotide.

The polynucleotide of interest can be inserted into the target chromosomal region by any suitable method, including, e.g., by gene targeting methods, such as homologous recombination, or by random insertion methods where transformed cells are subsequently screened for insertion into the desired chromosomal site. Chromosome engineering methods are discussed in more detail below, e.g., in the section on transgenic animals. By the phrase “spleen, lymphoid, and/or reticuloendothelial gene complex,” it is meant the region of the chromosome in which the cluster of genes, e.g., TMD1030 (XM_{—}166853), TMD1029 (XM_{—}166854), TMD1028 (XM_{—}166855), and TMD0621 (XM_{—}166205), of the present invention are located. Inserting an expressible polynucleotide (e.g., a polynucleotide operably linked to a promoter sequence) into this region confers the tissue expression selectivity which is characteristic of the gene cluster. Any polynucleotide of interest can be inserted into the chromosomal region, including, e.g., polynucleotides encoding polypeptides, antisense polynucleotides, etc.

A cell comprising a polynucleotide inserted into the target chromosomal location can be utilized in vitro or in vivo, e.g., in a transgenic animal. The cell is grown under conditions which are suitable to achieve polynucleotide expression These conditions depend upon the cell's environment, e.g., tissue culture cell, or in the form of a transgenic animal.

Pancreas Membrane Protein Genes

The present invention relates to all facets of pancreas membrane protein genes, polypeptides encoded by them, antibodies and specific binding partners thereto, and their applications to research, diagnosis, drug discovery, therapy, clinical medicine, forensic science and medicine, etc. The polynucleotides and polypeptides are useful in variety of ways, including, but not limited to, as molecular markers, as drug targets, and for detecting, diagnosing, staging, monitoring, prognosticating, preventing or treating, determining predisposition to, etc., diseases and conditions, such as pancreatic cancer, diabetes, pancreatitis, and other disorders especially relating to the pancreas and the functions its performs. The identification of specific genes, and groups of genes, expressed in pathways physiologically relevant to pancreas tissue permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

The function, structure, and diseases of the pancreas were described previously. The polynucleotides, polypeptides, and ligands thereto, of the present invention can be used to identify, detect, stage, determine the presence of, prognosticate, treat, study, etc., diseases and conditions of pancreas. These include, but are not limited to, e.g., acute and chronic pancreatitis, pancreatic abscess, pancreatic pseudocyst, nonalcoholic pancreatitis, alcoholic pancreatitis, classic acute hemorrhagic pancreatitis, chronic calcifying pancreatitis, familial hereditary pancreatitis, carcinomas of the pancreas, primary (idiopathic) diabetes (e.g., Type I (insulin dependent diabetes mellitus, IDDM) [insulin deficiency, beta cell depletion], Type II (non-insulin dependent diabetes mellitus, NIDDM) [insulin resistance, relative insulin deficiency, mild beta cell depletion]), nonobese NIDDM, obese NIDDM, maturity-onset diabetes of the young (MODY), islet cell tumors, diffuse hyperplasia of the islets of Langerhans, benign adenomas, malignant islet tumors, hyperfunction of the islets of Langerhans, hyperinsulinism and hypoglycemia, Zollinger-Ellison syndrome, beta cell tumors (insulinoma), alpha cell tumors (glucagonoma), delta cell tumors (somatostatinoma), vipoma (diarrheogenic islet cell tumor), pancreatic cancers, pancreatic carcinoid tumors, multihormonal tumors, multiple endocrine neoplasia (MEN), MEN I (Wermer syndrome), MEN II (Sipple syndrome), MEN III or IIb, pancreatic endocrine tumors, etc.

For example, five different pancreatic tumor samples were examined (Nos. 1, 2, 3, 4, and 5). TMD0639 was up-regulated in about 1/5 pancreatic cancers (No. 4), TMD0645 was up-regulated in about 3/5 pancreatic cancers (Nos. 2, 3, and 5), and TMD1127 was up-regulated in about 2/5 pancreatic cancers (Nos. 1 and 4). These results indicate that the probes can be used in combination in order to maximize the detection of different types of pancreatic cancers and tumors. Thus, a sample from a patient can be assesses for expression of both TMD0645 and TMD1127 to increase the probability that the pancreas cancer will be detected.

In view of their selectivity and display on the cell surface, the membrane proteins of the present invention are useful targets for histological, diagnostic, and therapeutic applications relating to the cells (e.g., pancreatic progenitor, exocrine, endocrine, acinar, islet, alpha, beta, delta, F, D1, enterochromaffin, etc.) in which they are expressed. Antibodies and other protein binding partners (e.g., ligands, aptamers, small peptides, etc.) can be used to selectively target agents to a tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. They can also be used to detect metastatic cells in biopsies and other tissue samples. The genes and polypeptides encoded thereby can also be used in tissue engineering to identify tissues as they appear during the differentiation process, to target tissues, to modulate tissue growth (e.g., from starting stem cell populations), etc. Useful antibodies or other binding partners include those that are specific for parts of the polypeptide which are exposed extracellularly as indicated in Table 21. Any of the methods described above and below can be accomplished in vivo, in vitro, or ex vivo.

When expression is described as being “predominantly”. in a given tissue, this indicates that the gene's mRNAs levels are highest in this tissue as compared to the other tissues in which it was measured. Expression can also be 'selective,” where expression is observed. By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in that tissue when compared to other tissue-types.

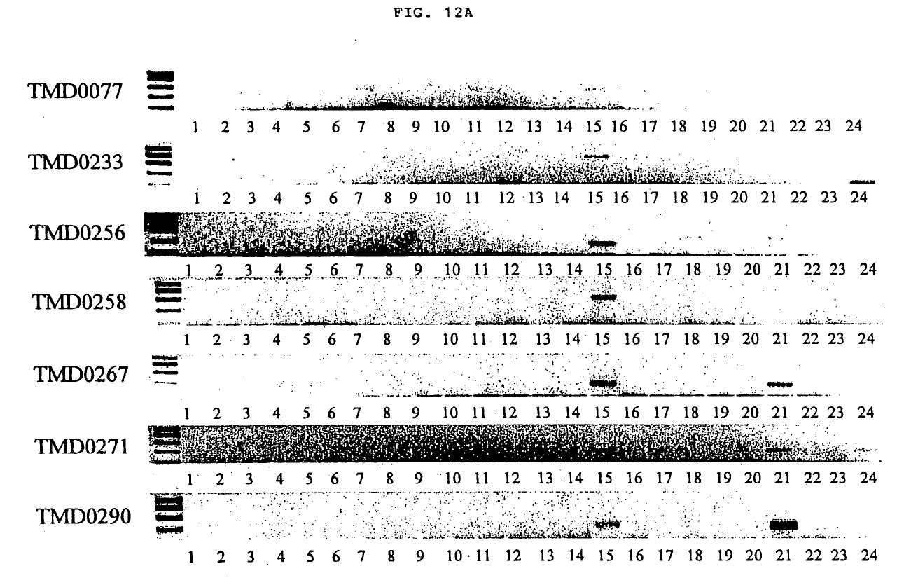

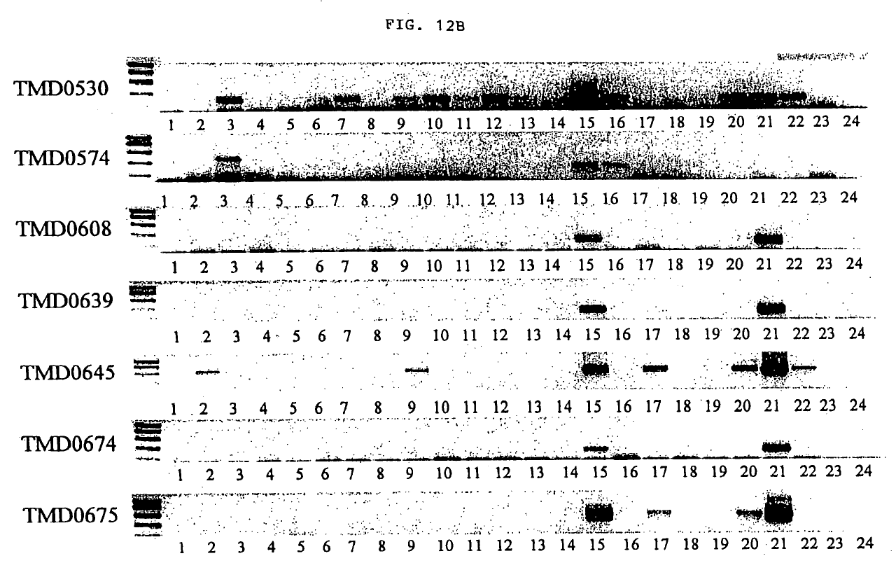

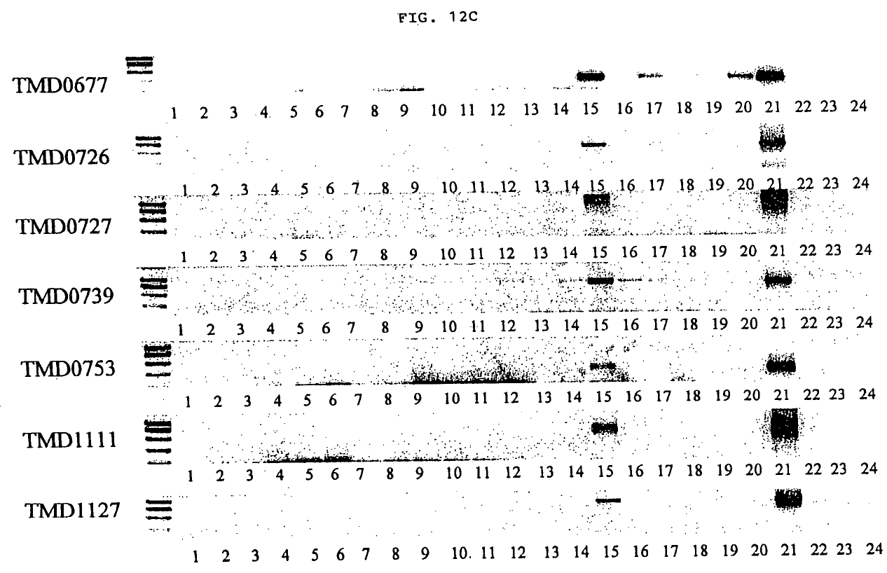

Table 20 is a summary of the genes of the present invention which are expressed selectively and/or predominantly in pancreas tissue. FIG. 12 is an illustration of these expression patterns. Each gene is associated with a Clone ID and Accession Number (“ACCN”). The Clone ID is an arbitrary identification number for the clone, and the accession number is the number by which it is listed in GenBank. Although specific sequences are disclosed herein, and listed in GenBank by an accession number), the present invention includes all forms of the gene, including polymorphisms, allelic variations, SNPs, splice variants, and any full-length versions when the disclosed or Genbank version is partial. For convenience, these genes, and their homologs in other species, are referred to throughout the disclosure in shorthand as “the genes of Table 20,” “a gene of Table 20,” “polynucleotides of Table 20,” “polypeptides of Table 20,” etc., because Table 20 contains a listing of the genes by accession number and clone ID.

The expression patterns of the selectively and/or predominantly expressed polynucteotides disclosed herein can be described as a “fingerprint” in that they are a distinctive pattern displayed by pancreas tissue. Just as with a fingerprint, an expression. pattern can be used as a unique identifier to characterize the status of a tissue sample. The list of expressed sequences disclosed herein provides an example of such a tissue expression profile. It can be used as a point of reference to compare and characterize samples. Tissue fingerprints can be used in many ways, e.g., to classify an unknown tissue, to determine the origin of metastatic cells, to assess the physiological status of a tissue, to determine the effect of a particular treatment regime on a tissue, to evaluate the toxicity of a compound on a tissue of interest, etc.

For example, the pancreas-selective polynucleotides disclosed herein represent the configuration of genes expressed by a normal pancreas tissue. To determine the effect of a toxin on a tissue, a sample of tissue can be obtained prior to toxin exposure (“control”) and then at one or more time points after toxin exposure (“experimental”). An array of pancreas-selective probes can be used to assess the expression patterns for both the control and experimental samples. As discussed in more detail below, any suitable method can be used. For instance, a DNA microarray can be prepared having a set of pancreas-selective genes arranged on to a small surface area in fixed and addressable positions. RNA isolated from samples can be labeled using reverse transcriptase and radioactive nucleotides, hybridized to the array, and then expression levels determined using a detection system. Several kinds of information can be extracted: presence or absence of expression, and the corresponding expression levels. The normal tissue would be expected to express substantially all the genes represented by the tissue-selective probes. The various experimental conditions can be compared to it to determine whether a gene is expressed, and how its levels match up to the normal control.

While the expression profile of the complete gene set represented by the sequences disclosed here may be most informative, a fingerprint containing expression information from less than the full collection can be useful, as well. In the same way that an incomplete fingerprint may contain enough of the pattern of whorls, arches, loops, and ridges, to identify the individual, a cell expression fingerprint containing less than the full complement may be adequate to provide useful and unique identifying and other information about the sample. Moreover, because of heterogeneity of the population, as well differences in the particular physiological state of the tissue, a tissue's “normal” expression profile is expected to differ between samples, albeit in ways that do not change the overall expression pattern. As a result, a complete match with a particular tissue expression profile, as shown herein, is not necessary.

The present invention relates to methods of detecting pancreas cells, comprising one or more of the following steps, e.g., contacting a sample comprising cells with a polynucleotide specific for a gene of Table 20, or a mammalian homolog thereof, under conditions effective for said polynucleotide to hybridize specifically to said gene, and detecting specific hybridization. Detecting can be accomplished by any suitable method and technology, including, e.g., any of those mentioned and discussed below, such as Northern blot and PCR. Specific polynucleotides include the primer sequences shown in Table 23, and complements thereto.

Detection can also be achieved using binding partners, such as antibodies (e.g., monoclonal or polyclonal antibodies) that specifically recognize polypeptides coded for by genes of the present invention. Thus, the present invention relates to methods of detecting a pancreas cell, comprising, one or more the following steps, e.g. contacting a sample comprising cells with a binding partner (e.g. an antibody, an Fab fragment, a single-chain antibody, an aptamer) specific for a polypeptide coded for by a polypeptide of Table 20, or a mammalian homolog thereof, under conditions effective for said binding partner bind specifically to said polypeptide, and detecting specific binding. Protein binding assays can be accomplished routinely, e.g., using immunocytochemistry, ELISA format, Western blots, etc. Useful epitopes include those exposed to the surface.

As indicated above, binding partners can be used to deliver agents specifically to the pancreas, e.g., for diagnostic, therapeutic, and prognostic purposes. Methods of delivering an agent to a pancreas cell can comprise, e.g., contacting a pancreas cell with an agent coupled to a binding partner specific for a polypeptide coding for a gene of Table 20, whereby said agent is delivered to said cell. Any type of agent can be used, including, therapeutic and imaging agents. Contact with the pancreas can be achieved in any effective manner, including by administering effective amounts of the agent to a host orally, parentally, locally, systemically, intravenously, etc. The phrase “an agent coupled to binding partner” indicates that the agent is associated with the binding partner in such a manner that it can be carried specifically to the target site. Coupling includes, chemical bonding, covalent bonding, noncovalent bonding (where such bonding is sufficient to carry the agent to the target), present in a liposome or in a lipid membrane, associated with a carrier, such as a polymeric carrier, etc. The agent can be directly linked to the binding partner, or via chemical linkers or spacers. Any cell expressing a polypeptide coded for by a gene of Table 20 can be targeted, including, e.g., pancreatic progenitor, exocrine, endocrine, secretory, acinar, islet, alpha, beta, delta, F, D1, enterochromaffin, etc.

Imaging of specific organs can be facilitated using tissue selective antibodies and other binding partners that selectively target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a binding partner. Ultrasound contrast agents combined with binding partners, such as antibodies, are described in, e.g., U.S. Pat. Nos. 6,264,917, 6,254,852, 6,245,318, and 6,139,819. NIU contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate a partner with an agent for any desired purpose. See, Bruehineier et al., Nucl. Med. Biol., 29:321-327, 2002, for imaging pancreas using labeled receptor ligands. Antibodies and other ligands to receptors of the present invention can be used analogously.

A pancreas cell (see above for examples of pancreas cell types) can also be modulated in accordance with the present invention, e.g., by methods of modulating a pancreas cell, comprising, e.g., contacting said cell with an agent effective to modulate a gene of Table 20, or the biological activity of a polypeptide encoded thereby (e.g., SEQ D NO 215, 217, 219, 221, 223, 225, 227, 229, 231, 233, 235, 237, 239, 241, 243, 245, 247, 249, 251, 253, and 255), or a mammalian homolog thereof, whereby said pancreas cell is modulated. Modulation as used throughout includes, e.g., stimulating, increasing, agonizing, activating, amplifying, blocking, inhibiting, reducing, antagonizing, preventing, decreasing, diminishing, etc.

An activity or function of the pancreas cell can be modulated, including, e.g., regulation of blood sugar, modulation of all aspects of the various secreted polypeptides (hormones, enzymes, etc.) produced by the pancreas, ligand-binding, exocytosis, amylase (and any of the other 20 or so digestive enzymes produced by the pancreas) secretion, autocrine responses, apoptosis (e.g., in the survival of beta-islet cells), etc.

The present invention also relates to polypeptide detection methods for assessing pancreas function, e.g., methods of assessing pancreas function, comprising, detecting a polypeptide coded for by a gene of Table 20, fragments thereof, polymorphisms thereof, in a body fluid, whereby the level of said polypeptide in said fluid is a measure of pancreas function. Pancreas function tests are usually performed to determine whether the pancreas is functioning normally as a way of diagnosing pancreas disease. Various tests are commonly used, including, e.g., assays for the presence of pancreatic enzymes in body fluids (e.g., amylase, serum lipase, serum trypsin-like immuoreactivity), studies of pancreatic structure (e.g., using x-ray, sonography, CT-scan, angiography, endoscopic retrograde cholangiopancreatography), and tests for pancreatic function (e.g., secretin-pancreozymin (CCK) tst, Lundh meal test, Bz-Ty-PABA test, chymotrypsin in feces, etc). Detection of a polypeptide coded for by a gene of Table 20 provides an additional assessment tool, especially in diseases such as pancreatitis and pancreatic cancer where pancreatic markers can appear in the blood, stool, urine, and other body fluids. As with the other tests, elevated levels of said polypeptide in blood, or other fluids, can indicate impaired pancreas function. Values can be determined routinely, as they are for other markers, such as those mentioned above. Detecting can be performed routinely (see below), e.g., using an antibody which is specific for said polypeptide, by RIA, ELISA, or Western blot, etc., in analogy to the tests for pancreatic enzymes in body fluids.

Promoter sequences obtained from genes of the present invention can be utilized to selectively express heterologous genes in pancreas cells. Methods of expressing a heterologous polynucleotide in pancreas cells can comprise, e.g., expressing a nucleic acid construct in pancreas cells, said construct comprising a promoter sequence operably linked to said heterologous polynucleotide, wherein said promoter sequence is selected SEQ ID NO 258, 261, 262, 265-267, 270-272, 275, 278, 279, 282-284, 287, 290-293, 296, 297, 303, 306, 309-314, 317-320, 323-326, 329, 332-333, 336-338, 341, and 344 as shown in Table 23. In addition to the cell lines mentioned below, the construct can be expressed in primary cells or in established cell lines.

The genes and polypeptides of Table 20 can be used to identify, detect, stage, determine the presence of, prognosticate, treat, study, etc., diseases and conditions of the pancreas as mentioned above. The present invention relates to methods of identifying a pancreatic disease or pancreatic disease-susceptibility, comprising, e.g., determining the association of a pancreatic disease or pancreatic disease-susceptibility with a nucleotide sequence present within the pancreatic gene complex. An association between a pancreas disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target.

Human linkage maps can be constructed to establish a relationship between the cytogenetic locus as shown in Table 22 and a pancreatic disease or condition. Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype.

Nucleic Acids

A mammalian polynucleotide, or fragment thereof, of the present invention is a polynucleotide having a nucleotide sequence obtainable from a natural source. When the species name is used, e.g., a human, it indicates that the polynucleotide or polypeptide is obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,”. it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc.

The polynucleotides described herein can be partial sequences that correspond to full-length, naturally-occurring transcripts. The present invention includes, as well, full-length polynucleotides that comprise these partial sequences, e.g., genomic DNAs and polynucleotides comprising a start and stop codon, a start codon and a polyA tail, a transcription start and a polyA tail, etc. These sequences can be obtained by any suitable method, e.g., using a partial sequence as a probe to select a full-length cDNA from a library containing full-length inserts. A polynucleotide which “codes without interruption” refers to a polynucleotide having a continuous open reading frame (“ORF”) as compared to an ORF which is interrupted by introns or other noncoding sequences.

Polynucleotides and polypeptides can be excluded as compositions from the present invention if; e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

As explained in more detail below, a polynucleotide sequence of the invention can contain the complete sequence as shown herein, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions (including both 5′ and 3′ regions, as well introns) can be identified upstream or downstream of coding and expressed RNAs, and assayed routinely for activity, e.g., by joining to a reporter gene (e.g., CAT, GFP, alkaline phosphatase, luciferase, galatosidase). A promoter obtained from a tissue selective gene can be used, e.g., in gene therapy to obtain tissue-specific expression of a heterologous gene (e.g., coding for a therapeutic product or cytotoxin). 5′ and 3′ sequences (including, UTRs and introns) can be used to modulate or regulate stability, transcription, and translation of nucleic acids, including the sequence to which is attached in nature, as well as heterologous nucleic acids.

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complimentarily, e.g., base-pairing between nucleotides, such as A-T, G-C, etc. The invention thus also relates to polynucleotides, and their complements, which hybridize to a polynucleotide comprising a nucleotide sequence as set forth herein and genomic sequences thereof A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complimentarily with the nucleotide sequences set forth in herein and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complimentarily, between the sequences. The present invention particularly relates to polynucleotide sequences which hybridize to the nucleotide sequences set forth in the attached sequence disclosure or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65° C.). As the homology drops, lower-washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, fornamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a polynucleotide probe in a hybridization solution containing, e.g., about 5×SSC, 0.1-0.5% SDS, 100 μg/ml denatured salmon sperm DNA and 50% formamide, at 42° C., or hybridizing at 42° C. in 5×SSPE, 0.1-0.5% SDS, and 50% formamide, 100 μ/ml denatured salmon sperm DNA, and washing at 65° C. in 0.1% SSC and 0.1% SDS.

Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a ¹% SDS solution at 42° C. Whereas high stringency washes can allow for, e.g., less than 10%, less than 5% mismatch, etc., reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, or 97%, nucleotide complimentarily between the probe (e.g., a short polynucleotide of the sequences disclosed herein or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. BLAST can be used to calculate amino acid sequence identity, amino acid sequence homology, and nucleotide sequence identity. These calculations can be made along the entire length of each of the target sequences which are to be compared.

After two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100(1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence described herein, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8400, 8-300, 8-150, 8-100, 8-75, 7-50, 10-25, 14-16, at least about 8, at least about 10, 11, 12, 13, 14, 15, 16, 17, 18, 19, 20, 21, 22, 23, 24, 25, 26, or more, etc. The polynucleotides can have non-naturally-occurring nucleotides, e.g., inosine, AZT, 3TC, etc. The polynucleotides can have 100% sequence identity or complimentarily to a sequence disclosed herein, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for tissue selective genes, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample and distinguish them from non-target genes. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, e.g., in the nucleotide sequences of the present invention, and which is characteristic of that target sequence, and substantially no non-target sequences. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complimentarily) to the probe, but less stringent conditions can also be used. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of pancreas, or kidney, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, sstNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfarnate, sulfamide, phosphorothioate, phosphoramidate, methylphosphonate, carbarnate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²p, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting tissue selective genes. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci, 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Detection can be accomplished in combination with polynucleotide probes for other genes, e.g., genes which are expressed in other disease states, tissues, cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, breast, fat, adult and embryonic stem cells, etc.

Polynucleotides can be used in wide range of methods and compositions, including for detecting, diagnosing, staging, grading, assessing, prognosticating, etc. diseases and disorders associated with tissue selective genes, for monitoring or assessing therapeutic and/or preventative measures, in ordered arrays, etc. Any method of detecting genes and polynucleotides can be used; certainly, the present invention is not to be limited how such methods are implemented.

Along these lines, the present invention relates to methods of detecting polynucleotides of the present invention in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is described herein, a polynucleotide having, e.g., about 70%, 80%, 85%, 90%, 95%, 99%, or more sequence identity thereto, effective or specific fragments thereof, or comnplements thereto. The detection method can be applied to any sample, e.g., cultured primary, secondary, or established cell lines, tissue biopsy, blood, urine, stool, cerebral spinal fluid, and other bodily fluids, for any purpose.

Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complimentarily. The effective conditions are selected such that the probe hybridizes to a preselected and/or definite target nucleic acid in the sample. For instance, if detection of a polynucleotide set forth herein is desired, a probe can be selected which can hybridize to such target gene under high stringent conditions, without significant hybridization to other genes in the sample. To detect homologs of a polynucleotide set forth in herein, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain, e.g., kidney, pancreas, or immune cells. The approaches can be combined, and one or-more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Methods of Identifying Polymorphisms, Mutations, etc.

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from subjects with diseases that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from the polynucleotides of the present invention is used as a probe. The selected mutant alleles, SNPs, polymorphisms, etc., can be used diagnostically to determine whether a subject has, or is susceptible to a disorder associated with tissue selective genes disclosed herein, as well as to design therapies and predict the outcome of the disorder. Methods involve, e.g., diagnosing a disorder or determining susceptibility to a disorder, comprising, detecting the presence of a mutation in a gene represented by a polynucleotide selected from the sequences disclosed herein. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in tissue selective genes, comprising, e.g., comparing the structure of: genomic DNA comprising all or part of a tissue selective gene, mRNA comprising all or part of a tissue selective gene, cDNA comprising all or part of a tissue selective gene, or a polypeptide comprising all or part of a tissue selective gene, with the structure the polynucleotides set forth herein. The methods can be carried out on a sample from any source, e.g., cells, tissues, body fluids, blood, urine, stool, hair, egg, sperm, cerebral spinal fluid, biopsy samples, serum, etc.

These methods can be implemented in many different ways. For example, “comparing the structure” steps include, but are not limited to, comparing restriction maps, nucleotide sequences, amino acid sequences, RFLPs, Dnase sites, DNA methylation fingerprints (e.g., U.S. Pat. No. 6,214,556), protein cleavage sites, molecular weights, electrophoretic mobilities, charges, ion mobility, etc., between standard and a test genes. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated pglynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), linker-scanning (McKnight and Kingsbury, Science, 217:316-324, 1982), directed using PCR, recursive ensemble mutagenesis (Arkin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhlmann, Gene, 71:29-40, 1988). For directed mutagenesis methods, analysis of the three-dimensional structure of the polypeptide can be used to guide and facilitate making mutants which effect polypeptide activity. Sites of substrate enzyme interaction or other biological activities can also be determined by analysis of crystal structure as determined by such techniques as nuclear magnetic resonance, crystallography or photoaftinity labeling. See, for example, de Vos et al., Science 255:306-312, 1992; Smith et al., J. Mol. Biol. 224:899-904, 1992; Wlodaver et al., FEBS Lett. 309:59-64, 1992.

In addition, libraries of genes and fragments thereof can be used for screening and selection of genes variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vector. By this method, expression libraries can be made comprising “mutagenized” tissue selective genes. The entire coding sequence or parts thereof can be used.

Polynucleotide Expression, Polypeptides Produced Thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CVI, BHK, CHO, Hela, LTK, NIH 3T3, insect cells, such as Sf9 (S. frugipeda) and Drosophila, bacteria, such as E. coli, Streptococcus, bacillus, yeast, such as Sacharomyces, S. cerevisiae, fungal cells, plant cells, embryoric or adult stem cells (e.g., mammalian, such as mouse or human),

immune system cell lines, HH (ATCC CRL 2105), MOLT-4 (ATCC CRL 1582), MJ (ATCC CRL-8294), SK7 (ATCC HB-8584), SK8 (ATCC HB-8585), HMI (HB-8586), H9 (ATCC HTB-176), HuT 78 (ATCC TIB-161), HuT 102 (ATCC TIB-162), Jurkat,

B-cell lines, B-cell precursor lines, NALM-36, B-cell and other lymphocyte lines immortalized with Epstein-Barr virus (transformed B lymphoblastoid), stromal cell lines, myelomas, HBM-Noda, WEH1231,

reticuloendothelial cells, endothelial cells, white blood cells, macrophages, antigen-resenting cells, lymphocytes, GDM-1 (ATCC CRL-2627), THP-1 (ATCC TIB-202), HL-60 (ATCC CCL-240), and derivatives thereof, including primary and established cell lines thereof,

kidney cell lines, 293, G-402 (ATCC CRL-1440), ACHN (ATCC CRL-161 1), Vero (ATCC CCL-81), 786-O (ATCC CRL-1932), 769-P (ATCC CRL-1933), CCD 1103 KIDTr (ATCC CRL-2304), CCD 1105 KIDTr (ATCC CRL-2305), Hs 835.T (ATCC CRL-7569), Hs 926.T (ATCC CRL-7678), Caki-1 (ATCC HTB-46), Caki-2 (ATCC HTB47), SW 839 (ATCC HTB-49), LLC-MK2 (ATCC CCL-7), BHK-21 (ATCC CCL-10), MDCK, CV-1, (ATCC CRL-1573), KNRK (ATCC CRL-1569), NRK49F (ATCC CRL-1570), A-704 (ATCC HTB-45), etc., established and primary kidney cells,

pancreas cell lines, insulinoma cell lines, INS-Hi, MIN6N8, RIN 1046-38, RIN-5AH, RIN-A12, RINm5F, capan-1, capan-2, hYIA PaCa-2 (ATCC CRL-1420), PANC-1 (ATCC CRL-1469), AsPC-1 (ATCC CRL-1682), SU-86.86 (ATCC CRL-1837), CFPAC-1 (ATCC CRL-1918), HPAF-1 (ATCC CRL-1937), TGP61 (ATCC CRL-2135) and other TGP lines, SW 1990 (ATCC CRL-2172), Mpanc-96 (ATCC CRL-2380), MS1 VEGF (ATCC CRL-2460), Beta-TC-6 (ATCC CRL-11506), LTPA (ATCC CRL-2389), 266-6 (ATCC CRL-2151), MS1 (ATCC CRL-2779), SVR (ATCC CRL-2280), NIT-2 (ATCC CRL-2364), alphaTC1 Clone 9 (ATCC CRL-2350), ATCC CRL-1492, BxPC-3 (ATCC CRL-1687), HPAC (ATCCCRL-2119), U.S. Pat. Nos. 6,110,743, 5,928,942, 5,888,816, 5,888,705, and 5,723,333, etc., established and primary pancreas cells (e.g., according to Hellerstrom et al., Diabetes, 28:769-76, 1979),

retinal cell lines, RF/6A (CRL 1780), ARPE-19 (CRL-2302), ARPE-19/HPV-16 (CRL-2502), Y79 (HTB-18), WERI-Rb-1 (HTB-169), RPE-J (CRL-2240), SO-Rb50 (retinoblastoma cell line), RBL, HER-Xho1-CC2, WERI-Rb24 (Sery et al., J. Pediatr. Ophthalmol. Strabismus, 4:212-217, 1990), WERI-Rb27 (Sery et al., J. Pediatr. Ophthalmol. Strabismnus, 4:212-217, 1990), HXO-Rb44, fetal retina cells, retinoblastoma cells, choroidal endothelial cells (e.g., Chor 55), etc., established and primary retinal cells (For other cell lines and methods thereof, see, also, Griege et al, Differentiation, 45:250-7, 1990; Bernstein et al., Invest. Ophthalmol. Vis. Sci., 35:3931-3937, 1994; Howes et al., Invest Ophthalmol. Vis. Sci., 35:342-351, 1994).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in herein or it can contain degenerate codons coding for the same amino acid sequences. For instance, it may be desirable to change the codons in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6×His, maltose binding protein, chitinase, etc) and then purify by anti-tag antibody-conjugated affinity chromatography.

The present invention also relates to specific-binding partners. These include antibodies which are specific for polypeptides encoded by polynucleotides of the present invention, as well as other binding-partners which interact with polynucleotides and polypeptides of the present invention. Protein-protein interactions between polypeptides and binding partners can be identified using any suitable methods, e.g., protein binding assays (e.g., filtration assays, chromatography, etc.), yeast two-hybrid system (Fields and Song, Nature, 340: 245-247, 1989), protein arrays, gel-shift assays, FRET (fluorescence resonance energy transfer) assays, etc. Nucleic acid interactions (e.g., protein-DNA or protein-RNA) can be assessed using gel-shift assays, e.g., as carried out in U.S. Pat. No. 6,333,407 and 5,789,538.

Antibodies, e.g., polyclonal, monoclonal, recombinant, chimeric, humanized, single-chain, Fab, and fragments thereof, can be prepared according to any desired method. Antibodies, and immune responses, can also be generated by administering naked DNA See, e.g., U.S. Pat. Nos. 5,703,055; 5,589,466; 5,580,859. Antibodies can be used from any source, including, goat, rabbit, mouse, chicken (e.g., IgY; see, Duan, W0/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA. Antibodies can be prepared against specific epitopes or domains.

Antibodies can also be humanized, e.g., where they are to be used therapeutically. Methods for obtaining human antibodies, e.g., from transgenic mice are described, e.g., in Green et al., Nature Genet. 7:13 (1994); Lonberg et al., Nature 368:856 (1994); and Taylor et al., Int. Immunol. 6:579 (1994). Antibody fragments of the present invention can be prepared by any suitable method, Fab and Fc fragments single-chain antibodies can also be used. Another form of an antibody fragment is a peptide coding for a single complimentarily-determining region (CDR). CDR peptides (“minimal recognition units”) can be obtained by constructing genes encoding the CDR of an antibody of interest.

The term “antibody” as used herein includes intact molecules as well as fragments thereof, such as Fab, F(ab′)2, and Fv which are capable of binding to an epitopic determinant present in Bin1 polypeptide. Such antibody fragments retain some-ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to polypeptides of the present invention can be prepared using an intact polypeptide or fragments containing small peptides of interest as the immunizing antigen. For example, it may be desirable to produce antibodies that specifically bind to the N- or C-terminal domains of the tissue selective polypeptides of the present invention. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Methods of Detecting Polypeptides

Polypeptides coded for by genes of the present invention can be detected, visualized, determined, quantitated, etc. according to any effective method, useful methods include, e.g., but are not limited to, immunoassays, RIA (radioimmunassay), ELISA, (enzyme-linked-immunosorbent assay), immunoflourescence, flow cytometry, histology, electron microscopy, light microscopy, in situ assays, immunoprecipitation, Western blot, etc.

Immunoassays may be carried in liquid or on biological support. For instance, a sample (e.g., blood, serum, stool, urine, cells, tissue, cerebral spinal fluid, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylarides, and magnetite. A support material can have any structural or physical configuration. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersyille, Md.); Voller, A. et al., 1978, J. Clin. Pathol. 31, 507-520; Butler, J. E., 1981, Meth. Enzymol. 73, 482-523; Maggio; E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoarnylase and acetylcholinesterase. The detection can be accomplished by calorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect peptides through the use of a radioimmunoassay (RIA). See, e.g., Weintraub, B., Principles of Radioimnnunoassays, Seventh Training Course on Radio ligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerthrin, phycocyanin, allophycocyanin, o-phthaldehyde and fluorescamine. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic

The present invention also relates to methods and compositions for diagnosing a disorder, or determining susceptibility to a disorder, using polynucleotides, polypeptides, and specific-binding partners of the present invention to detect, assess, determine, etc., a tissue selective gene. In such methods, the gene can serve as a marker for the disorder, e.g., where the gene, when mutant, is a direct cause of the disorder-, where the gene is affected by another gene(s) which is directly responsible for the disorder, e.g., when the gene is part of the same signaling pathway as the directly responsible gene; and, where the gene is chromosomally linked to the gene(s) directly responsible for the disorder, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The phrase “diagnosing” indicates that it is determined whether the sample has the disorder. A “disorder” means, e.g., any abnormal condition as in a disease or malady. “Determining a subject's susceptibility to a disease or disorder” indicates that the subject is assessed for whether s/he is predisposed to get such a disease or disorder, where the predisposition is indicated by abnormal expression of the gene (e.g., gene mutation, gene expression pattern is not normal, etc.). Predisposition or susceptibility to a disease may result when a such disease is influenced by epigenetic, environmental, etc., factors. Diagnosing includes prenatal screening where samples from the fetus or embryo (e.g., via amniocentesis or CV sampling) are analyzed for the expression of the gene.

By the phrase “assessing expression of a gene or polynucleotide,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

The genes and polypeptides of the present invention can be used to identify, detect, stage, determine the presence of, prognosticate, treat, study, etc., diseases and conditions as mentioned above. The present invention relates to methods of identifying a genetic basis for a disease or disease-susceptibility, comprising, e.g., determining the association of a disease or disease-susceptibility with a gene of the present invention. An association between a disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target Any region of the gene can be used as a source of the DNA marker, exons, introns, intergenic regions, etc.

Human linkage maps can be constructed to establish a relationship between a gene and a disease or condition Typically, polymorphic molecular markers (e.g., STRP's, SNP's, RFLP's, VNTR's) are identified within the region, linkage and map distance between the markers is then established, and then linkage is established between phenotype and the various individual molecular markers. Maps can be produced for an individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype. See, e.g., Kruglyak et al., Am. J. Hum. Genet., 58, 1347-1363, 1996; Matise et al., Nat. Genet., 6(4):384-90, 1994.

Assessing the effects of therapeutic and preventative interventions (e.g., administration of a drug, chemotherapy, radiation, etc.) on disorders is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving disease treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having a disorder, or, susceptible to such a disorder, comprising, e.g., detecting the expression levels of one or more tissue selective genes. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose a disorder.

The present invention also relates to methods of using binding partners, such as antibodies, to deliver active agents to the tissue (e.g., kidney or pancreas or an immune cells) for a variety of different purposes, including, e.g., for diagnostic, therapeutic, and research purposes. Methods can involve delivering or administering an active agent to the tissue, comprising, e.g., administering to a subject in need thereof, an effective amount of an active agent coupled to a binding partner specific for a tissue selective polypeptide, wherein said binding partner is effective to deliver said active agent specifically to the target tissue.

Any type of active agent can be used in combination with it, including, therapeutic, cytotoxic, cytostatic, chemotherapeutic, anti-neoplastic, anti-proliferative, anti-biotic, etc., agents. A chemotherapeutic agent can be, e.g., DNA-interactive agent, alkylating agent, antimetabolite, tubulin-interactive agent, hormonal agent, hydroxyurea, Cisplatin, Cyclophosphamide, Altretarnine, Bleomycin, Dactinomycin, Doxorubicin, Etoposide, Teniposide, paclitaxel, cytoxan, 2-methoxy-carbonyl-amnino-benzimidazole, Plicanycin, Methotrexate, Fluorouracil, Fluorodeoxyuridin, CB3717, Azacitidine, Floxuridine, Mercapyopurine, 6-Thioguanine, Pentostatin, Cytarabine, Fludarabine, etc. Agents can also be contrast agents useful in imaging technology, e.g., X-ray, CT, CAT, MRI, ultrasound, PET, SPECT, and scintographic.

An active agent can be associated in any manner with a binding partner which is effective to achieve its delivery specifically to the target. Specific delivery or targeting indicates that the agent is provided to the tissue, without being substantially provided to other tissues. This is useful especially where an agent is toxic, and specific targeting to the tissue enables the majority of the toxicity to be aimed at the tissue, with as small as possible effect on other tissues in the body. The association of the active agent and the binding partner (“coupling”) can be direct, e.g., through chemical bonds between the binding partner and the agent, or, via a linking agent, or the association can be less direct, e.g., where the active agent is in a liposome, or other carrier, and the binding partner is associated with the liposome surface. In such case, the binding partner can be oriented in such a way that it is able to bind to tissue selective polypeptide, e.g., exposed on the cell surface. Methods for delivery of DNA via a cell-surface receptor is described, e.g., in U.S. Pat. No. 6,339,139.

Identifying Agent Methods

The present invention also relates to methods of identifying agents, and the agents themselves, which modulate tissue selective genes. These agents can be used to modulate the biological activity of the polypeptide encoded for the gene, or the gene, itself. Agents which regulate the gene or its product are useful in variety of different environments, including as medicinal agents to treat or prevent disorders associated with genes and as research reagents to modify the function of tissues and cell.

Methods of identifying agents generally comprise steps in which an agent is placed in contact with the gene, its transcription product, its translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of tissue selective genes, a method can comprise, in any effective order, one or more of the following steps, e.g., contacting a gene (e.g., in a cell population) with a test agent under conditions effective for said test agent to modulate the expression of tissue selective genes, and determining whether said test agent modulates said genes. An agent can modulate expression of a tissue selective gene at any level, including transcription (e.g., by modulating the promoter), translation, and/or perdurance of the nucleic acid (e.g., degradation, stability, etc.) in the cell.

For modulating the biological activity of polypeptides, a method can comprise, in any effective order, one or more of the following steps, e.g., contacting a polypeptide (e.g., in a cell, lysate, or isolated) with a test agent under conditions effective for said test agent to modulate the biological activity of said polypeptide, and determining whether said test agent modulates said biological activity.

Contacting a gene or polypeptide with the test agent can be accomplished by any suitable method and/or means that places the agent in a position to functionally control expression or biological activity. Functional control indicates that the agent can exert its physiological effect through whatever mechanism it works. The choice of the method and/or means can depend upon the nature of the agent and the condition and type of environment in which the gene or polypeptide is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide which is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access, it can be determined whether the test agent modulates expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent. This effect includes, increase, decrease, block, inhibit, enhance, etc.

A test agent can be of any molecular composition, e.g., chemical compounds, biomolecules, such as polypeptides, lipids, nucleic acids (e.g., antisense), carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading to its down regulation on the surface of the cell. Such an effect does not have to be permanent, but can require the presence of the antibody to continue the down-regulatory effect. Antibodies can also be used to modulate the biological activity of a polypeptide in a lysate or other cell-free form.

Additional cell-based test systems suitable for the analysis of GPCR polypeptides are summarized in Marchese et al. (1999, Trends in Pharmacol. Sci. 20: 370-375) and comprise so-called “ligand screening assays.” For example in yeast cells the pheromon receptor can be replaced by a GPCR according to the invention. The effect of test substances on the receptor can be determined upon modulation of histidine synthesis, i.e. by growing in histidine-free medium. In addition using cells transfected with nucleic acids according to the invention it can be analyzed whether test substances mediate translocation of a detectable arrestins, for example of a arrestin-GFP-fusion protein. Moreover, it can be analyzed whether test substances mediate GPCR-mediated dispersion or aggregation of Xenopus laevis. melanophores. Another test system utilizes the universal adapter G-protein G alphal6, which mobilizes Ca.sup.2+. Other screening test systems are described in Lemer et al., supra; WO96/41169; U.S. Pat. No. 5,482,835; WO99/06535; EP 0 939 902; WO99/66326; WO98/34948; EP 0 863 214; U.S. Pat. No. 5,882,944 and U.S. Pat. No. 5,891,641.

Therapeutics

Selective polynucleotides, polypeptides, and specific-binding partners thereto, can be utilized in therapeutic applications, especially to treat diseases and conditions described herein. Useful methods include, but are not limited to, immunotherapy (e.g., using specific-binding-partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack a cancer or other diseased tissue, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheda toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vitro and in vivo to silence a gene when its expression contnbutes to a disease (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc. Therapeutic agents of the present invention can be administered in any form by any effective route, including, e.g., oral, parenteral, enteral, intraperitoneal, topical, transdermal (e.g., using any standard patch), intravenously, ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

In addition to therapeutics, per se, the present invention also relates to methods of treating a disease showing altered expression of a tissue selective gene, comprising, e.g., administering to a subject in need thereof a therapeutic agent which is effective for regulating expression of said gene and/or which is effective in treating said disease. The term “treating” is used conventionally, e.g., the management or care of a subject for the purpose of combating, alleviating, reducing, relieving, improving the condition of, etc., of a disease or disorder. By the phrase “altered expression,” it is meant that the disease is associated with a mutation in the gene, or any modification to the gene (or corresponding product) which affects its normal function. Thus, expression refers to, e.g., transcription, translation, splicing, stability of the mRNA or protein product, activity of the gene product, differential expression, etc.

Any agent which “treats” the disease can be used. Such an agent can be one which regulates the expression of a tissue selective gene. Expression refers to the same acts already mentioned, e.g. transcription, translation, splicing, stability of the mRNA or protein product, activity of the gene product, differential expression, etc. For instance, if the condition was a result of a complete deficiency of the gene product, administration of gene product to a patient would be said to treat the disease and regulate the gene's expression. Many other possible situations are possible, e.g., where the gene is aberrantly expressed, and the therapeutic agent regulates the aberrant expression by restoring its normal expression pattern.

Antisense

Antisense polynucleotide (e.g., RNA) can also be prepared from a polynucleotide according to the present invention. Antisense polynucleotide can be used in various ways, such as to regulate or modulate expression of the polypeptides they encode, e.g., inhibit their expression, for in situ hybridization, for therapeutic purposes, for making targeted mutations (in vivo, triplex, etc.) etc. For guidance on administering and designing anti-sense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708. An. antisense polynucleotides can be operably linked to an expression control sequence. A total length of about 35 bp can be used in cell culture with cationic liposomes to facilitate cellular uptake, but for in vivo use, preferably shorter oligonucleotides are administered, e.g. 25 nucleotides.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); 4,973,679; Sproat et al., “2′-O-Methyloligoribonucleotides: synthesis and applications,” Oligonucleotides and Analogs A Practical Approach, Eckstein (ed.), IRL Press, Oxford, 1991, 49-86; Iribarren et al., “2′O-Alkyl Oligoribonucleotides as Antisense Probes,” Proc. Natl. Acad. Sci. USA, 1990, 87, 7747-7751; Cotton et al., “2′-O-methyl, 2′-O-ethyl oligoribonucleotides and phosphorothioate oligodeoxynrbonucleotides as inhibitors of the in vitro U7 snRNP-dependent mRNA processing event,” Nucl. Acids Res., 1991, 19, 2629-2635.

Arrays

The present invention also relates to an ordered array of polynucleotide probes and specific-binding partners (e.g., antibodies) for detecting the expression of tissue selective genes or polypeptides encoded thereby, in a sample, comprising, one or more polynucleotide probes or specific binding partners associated with a solid support or in separate receptacles, wherein each probe is specific for a tissue selective gene or a specific-binding partner which is specific for a polypeptide.

The phrase “ordered array” indicates that the probes are arranged in an identifiable or position-addressable pattern, e.g., such as the arrays disclosed in U.S. Pat. Nos. 6,156,501, 6,077,673, 6,054 ,270, 5,723,320, 5,700,637, WO0991971 1, WO00023803. The probes are. associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Transgenic animals

The present invention also relates to trarsgenic animals comprising tissue selective genes, and homologs thereof (Methods of making transgenic animals, and associated recombinant technology, can be accomplished conventionally, e.g., as described in Transgenic Animal Technology, Pinkert et al., 2^{nd} Edition, Academic Press, 2002.) Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. The nucleic acid present in the construct or transgene can be naturally-occurring wild-type, polymorphic, or mutated. Where the animal is a non-human animal, its homolog can be used instead. Transgenic animals can have structural and/or functional defects in any of the tissues described herein, e.g., pancreas, kidney, retina, and immune cells, as well as having or being susceptible to any of the associated disorders or diseases mentioned herein.

Along these lines, polynucleotides of the present invention can be used to create transgenic animals, e.g. a non-human animal, comprising at least one cell whose genome comprises a functional disruption of one or tissue selective genes, or homologs thereof (e.g., a mouse homolog when a mouse is used). By the phrases “functional disruption” or functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amnino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the gene (or homolog thereof), etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which-comprises the functional disruption can also be referred to as a “knock-out” animal, since the biological activity of its gene has been “knocked-out.” Knock-outs can be homozygous or heterozygous.

For creating functionally disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, E.G. cells), and that genetically-modified cell (or nucleus) is used to create a whole organism. Nuclear transfer can be used in combination with homologous recombination technologies. For example, a gene locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a an exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and geriline animals, e.g., animals having somatic and/or germ cells comprising the engineered gene. Homozygous knock-out animals can be obtained from breeding heterozygous knock-out animals. See, e.g., U.S. Pat. No. 6,225,525.

The present invention also relates to non-human, transgenic animal whose genome comprises recombinant tissue selective nucleic acid (and homologs thereof) operatively linked to an expression control sequence effective to express said coding sequence in a target tissue. Such a transgenic animal can also be referred to as a “knock-in” animal since an exogenous gene has been introduced, stably, into its genome. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant gene has been stably integrated into the genome of the animal. The nucleic acid (e.g., a coding sequence) in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palirter et al., Cell, 41:343-345, 1985; Palmiter et al., Ann. Rev. Genet., 20:465-499, 1986; Askew et al., Mol. Cell. Bio., 13:4115-4124, 1993; Gaines et al. Nature, 373:523-527, 1995; Valancius and Smithies, Mol. Cell. Bio., 11:1402-1408, 1991; Staceyet al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256 1258, 1998. For guidance on recombinase excision systems, see, e.g., U.S. Pat. Nos. 5,626,159, 5,527,695, and 5,434,066. See also, Orban, P. C., et al., “Tissue and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1): 147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, New York, 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePanphilis et al., Bio Techniques, 6:662-680, 1988. Transgenic animals can be produced by the methods described in U.S. Pat. No. 5,994,618, and utilized for any of the utilities described therein

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving nucleic acid and/or polypeptide sequences from a computer-readable medium, comprising, one or more of the following steps in any effective order, e.g., selecting a cell or gene expression profile, e.g., a profile that specifies that said gene is differentially expressed in a tissue as described herein, and retrieving said differentially expressed nucleic acid or polypeptide.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type of gene or cell expression pattern he is interested in retrieving, e.g., he may require that the gene is differentially expressed in a tissue, or he may require that the gene is not expressed in blood, but must be expressed in pancreas. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data The database is information that has been stored in an appropriate storage medium, having a suitable computer-readable format. Once results are retrieved, they can be displayed in any suitable format, such as HTML.

For instance, the user may be interested in identifying genes that are differentially expressed in a pancreas or kidney. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying tissue selective polynucleotide or polypeptide sequences, or antibody specific thereto, in a printed or computer-readable medium (e.g., on the Web or Internet), accepting an offer to purchase said gene, polypeptide, or antibody.

Other

A polynucleotide, probe, polypeptide, antibody, specific-binding partner; etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found. Reference materials

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety, including U.S. Application Ser. No. 60/372,669 Apr. 16, 2003, 60/374,823 filed Apr. 24, 2002, 60/376,558 filed May 1, 2002, 60/381,366 20 filed May 20, 2002, 60/403,648 filed Aug. 16, 2002, 60/411,882 filed Sep. 20, 2002, and 60/424,336 filed Nov. 7, 2002.

TABLE 1

| | | Predominant | Other | |
|---|---|---|---|---|
| Clone ID | | sites of | expression | Cytogenetic |
| (gene code) | ACCN | expression | sites | locus |
| TMD0024 | XM_060945 | thymus | none | 1q22 |
| TMD1779 | XM_060946 | thymus | none | 1q22 |
| | | and PBL | | |
| TMD0884 | XM_060947 | thymus | skin and | 1q22 |
| | | | ovary | |
| TMD0025 | XM_060948 | thymus | none | 1q22 |
| TMD1780 | XM_089422 | thymus | none | 1q22 |
| TMD1781 | XM_089421 | PBL | thymus | 1q22 |
| TMD0304 | XM_060956 | bone marrow | testis | 1q22 |
| | | and muscle | | |
| TMD0888 | XM_060957 | bone marrow | lung, muscle | 1q22 |
| | | | and testis | |
| TMD0890 | XM_060959 | bone marrow | lung and PBL | 1q22 |

TABLE 2

| Clone ID | | | |
|---|---|---|---|
| (gene | | Protein | |
| code) | ACCN | seq length | Domain Description |
| TMD1779 | XM_060946 | 264 | Transmembrane domain: 26-48 |
| | | | Transmembrane domain: 55-77 |
| | | | Transmembrane domain: 92-114 |
| | | | Transmembrane domain: 134-156 |
| | | | Transmembrane domain: 197-219 |
| TMD0024 | XM_060945 | 268 | Transmembrane domain: 16-38 |
| | | | Transmembrane domain: 53-75 |
| | | | Transmembrane domain: 96-118 |
| | | | Transmembrane domain: 156-178 |
| | | | Transmembrane domain: 191-213 |
| | | | Transmembrane domain: 228-246 |
| TMD0025 | XM_060948 | 313 | Transmembrane domain: 29-51 |
| | | | Transmembrane domain: 58-77 |
| | | | Transmembrane domain: 92-114 |
| | | | Transmembrane domain: 135-157 |
| | | | Transmembrane domain: 197-219 |
| | | | Transmembrane domain: 240-262 |
| | | | Transmembrane domain: 272-294 |
| TMD0304 | XM_060956 | 319 | Transmembrane domain: 28-50 |
| | | | Transmembrane domain: 63-82 |
| | | | Transmembrane domain: 102-124 |
| | | | Transmembrane domain: 144-166 |
| | | | Transmembrane domain: 205-227 |
| | | | Transmembrane domain: 240-262 |
| | | | Transmembrane domain: 272-294 |
| TMD0884 | XM_060947 | 299 | Transmembrane domain: 20-42 |
| | | | Transmembrane domain: 54-76 |
| | | | Transmembrane domain: 91-113 |
| | | | Transmembrane domain: 126-148 |
| | | | Transmembrane domain: 183-205 |
| | | | Transmembrane domain: 226-248 |
| | | | Transmembrane domain: 258-277 |
| TMD0888 | XM_060957 | 312 | Transmembrane domain: 25-47 |
| | | | Transmembrane domain: 59-78 |
| | | | Transmembrane domain: 98-120 |
| | | | Transmembrane domain: 141-163 |
| | | | Transmembrane domain: 207-229 |
| | | | Transmembrane domain: 241-260 |
| | | | Transmembrane domain: 270-292 |
| TMD0890 | XM_060959 | 280 | Transmembrane domain: 26-48 |
| | | | Transmembrane domain: 122-144 |
| | | | Transmembrane domain: 180-202 |
| | | | Transmembrane domain: 215-237 |
| | | | Transmembrane domain: 252-269 |
| TMD1780 | XM_089422 | 491 | Transmembrane domain: 20-42 |
| | | | Transmembrane domain: 54-76 |
| | | | Transmembrane domain: 91-113 |
| | | | Transmembrane domain: 137-159 |
| | | | Transmembrane domain: 190-212 |
| | | | Transmembrane domain: 231-253 |
| | | | Transmembrane domain: 266-283 |
| | | | Transmembrane domain: 304-326 |
| | | | Transmembrane domain: 336-358 |
| | | | Transmembrane domain: 379-401 |
| | | | Transmembrane domain: 437-459 |
| TMD1781 | XM_089421 | 91 | Transmembrane domain: 63-85 |

TABLE 3

| | TMD0024 | TMD1779 | TMD0884 | TMD0884 | TMD1780 | TMD1781 | TMD0304 | TMD0888 |
|---|---|---|---|---|---|---|---|---|
| | XM_060945 | XM_060946 | XM_060947 | XM_060948 | XM_089422 | XM_089421 | XM_060956 | XM_060957 |
| TMD0024 | | | | | | | | |
| XM_060945 | | | | | | | | |
| TMD1779 | no significant | | | | | | | |
| XM_060946 | similarity | | | | | | | |
| TMD0884 | 74% (371 nt) | no significant | | | | | | |
| XM_060947 | | simIlarity | | | | | | |
| TMD0025 | 71% (222 nt) | 90% (605 nt) | 83% (54 nt) | | | | | |
| XM_060948 | 80% (73 nt) | | | | | | | |
| TMD1780 | 81% (114 nt) | 83% (71 nt) | 78% (90 nt) | 80% (84 nt) | | | | |
| XM_089422 | 74% (186 nt) | | | | | | | |
| | 79% (113 nt) | | | | | | | |
| | 77% (99 nt) | | | | | | | |
| TMD1781 | 91% (35 nt) | no significant | no significant | no significant | 77% (179 nt) | | | |
| XM_089421 | 77% (80 nt) | similarity | similarity | similarity | 82% (46 nt) | | | |
| TMD0304 | no significant | no significant | no significant | no significant | 84% (39 nt) | no significant | | |
| XM_060956 | similarity | similarity | similarity | similarity | | similarity | | |
| TMD0888 | no significant | no significant | no significant | 84% (38 nt) | no significant | no significant | 73% (241 nt) | |
| XM_060957 | similarity | similarity | similarity | | similarity | similarity | | |
| TMD0890 | no significant | no significant | no significant | no significant | no significant | no significant | no significant | 84% (39 nt) |
| XM_060959 | similarity | similarity | similarity | similarity | similarity | similarity | similarity | |

TABLE 4

| | TMD0024 | TMD1779 | TMD0884 | TMD0025 | TMD1780 | TMD1781 | TMD0304 | TMD0888 |
|---|---|---|---|---|---|---|---|---|
| | XP_060945 | XP_060946 | XP_060947 | XP_060948 | XP_089422 | XP_089421 | XP_060956 | XP_060957 |
| TMD0024 | | | | | | | | |
| XP_060945 | | | | | | | | |
| TMD1779 | 47% (200 aa) | | | | | | | |
| XP_060946 | | | | | | | | |
| TMD0884 | 62% (171 aa) | 36% (92 aa) | | | | | | |
| XP_060947 | | | | | | | | |
| TMD0884 | 63% (252 aa) | 73% (233 aa) | 46% (166 aa) | | | | | |
| XP_060948 | | | | | | | | |
| TMD1780 | 59% (261 aa) | 46% (227 aa) | 55% (165 aa) | 52% (300 aa) | | | | |
| XP_089422 | 59% (181 aa) | 46% (169 aa) | 47% (111 aa) | | | | | |
| TMD1781 | 40% (94 aa) | 35% (82 aa) | 52% (40 aa) | 37% (94 aa) | 51% (93 aa) | | | |
| XP_089421 | | | | | 49% (77 aa) | | | |
| TMD0304 | 40% (257 aa) | 37% (229 aa) | 36% (163 aa) | 39% (299 aa) | 39% (300 aa) | 34% (89 aa) | | |
| XP_060956 | | | | | | | | |
| TMD0888 | 49% (251 aa) | 37% (239 aa) | 41% (127 aa) | 40% (305 aa) | 45% (304 aa) | 47% (82 aa) | 50% (301 aa) | |
| XP_060957 | | | | | 43% (189 aa) | | | |
| TMD0890 | 41% (196) | 32% (132 aa) | 32% (156 aa) | 36% (179 aa) | 42% (200 aa) | 38% (72 aa) | 36% (196 aa) | 46% (196 aa) |
| XP_060959 | | | | | | | | |

TABLE 5

| | | | |
|---|---|---|---|
| CLONE ID | F-OLIGO | R-OLIGO | PROMOTER |

| | | | |
|---|---|---|---|
| TMD1779 | GGTCAATGAGACTGTGG | CTATCACTCCCAGTGTGGAA | CTCTTTCAGATTTAAATGGGCCAGACTTAGTTTTATGTGGTGCAGACATT |
| (SEQ ID NO | TGAGAGAGGTCATCT | GGAAACTGAAG | (SEQ ID NO 5) |
| 1-2 | (SEQ ID NO 3) | (SEQ ID NO 4) | |
| TMD0024 | CCACCTGCTCTCAGACA | GGCACCATAATTACCAGGAT | GAGTGCCAAATATATAAAGAGGTATGTTCAATGCAACATGTTAAATGCAA |
| (SEQ ID NO | CCAAGACC | GCTGAGG | (SEQ ID NO 10) |
| 6-7) | (SEQ ID NO 8) | (SEQ ID NO 9) | ACTCCTTAGATAAAAAAGGGCAGATTTATTAAAGAACCCTGATTTAATCA |
| | | | (SEQ ID NO 11) |
| TMD0025 | CCTGTTCACTCTGGGCA | CTGGTTGGAGGAGTGGAAG | TAATACTATGTAAAAATCCACTGGACTAGAATCAGCTGTCCTCATGTGCC |
| (SEQ ID NO | CCAATGC | GGCAG | (SEQ ID NO 19) |
| 12-13) | (SEQ ID NO 14) | (SEQ ID NO 15) | TACCTTTCTGTATATAAAAACATATAACTAATACACACACACTCATACAC |
| | | | (SEQ ID NO 16) |
| | | | CTTCAGAAGTATATAAATGAAGACTGGATACCAGCAAGACATACTGGATG |
| | | | (SEQ ID NO 17) |
| | | | CCCTTGGAGATATAAAAAGTTCCCAGTAAATAGATGTGTCCTCACATCTT |
| | | | (SEQ ID NO 18) |
| TMD0304 | CTCTATGTTCCCGCATGC | GCAAGGTGGAAATCCATGCA | AGACAGACGTTAAAAAATGACCAAACCTACAGAAAATATTTCCAGATAAT |
| (SEQ ID NO | GCACAG | ATCTCAG | (SEQ ID NO 24) |
| 20-21) | (SEQ ID NO 22) | (SEQ ID NO 23) | |
| TMD0884 | TGTCAATATCCTGGTGTT | CATCTACCCAGAACCTTTCT | GTCACTGCTGTATAAGCACGCAGTGCAAAGGAAATATTAAAACTAGAACC |
| (SEQ ID NO | CAGTGTGCTCC | CAGAGCCATC | (SEQ ID NO 29) |
| 25-26) | (SEQ ID NO 27) | (SEQ ID NO 28) | TTTCTTCATTTATAACATGAGGGGGCTTGGCTAGATATTTAACAGCCTGC |
| | | | (SEQ ID NO 30) |
| | | | GCTAGATATTTAACAGCCTGCCTGTATTGACCACTTATGCATCAGGAAAT |
| | | | (SEQ ID NO 31) |
| | | | ATTTGAGTTATGTATATGAGAGACTGGGTACATCACTTTTTACTTGTTTT |
| | | | (SEQ ID NO 32) |
| TMD0888 | GGAACTGGAGCCAGGTA | GGAGCAGAGGATCAGCAGG | ACACTGCAGTTATATAGGGTGGCCCAGGTAGTTGAGCTGGTGAAATTTGA |
| (SEQ ID NO | GCAGAATTCATC | AAGGTG | (SEQ ID NO 37) |
| 33-34) | (SEQ ID NO 35) | (SEQ ID NO 36) | GCACTGTGACATTAAAAGGATGGGGCATGGAGGAGAAACTAAAGTTGGAG |
| | | | (SEQ ID NO 38) |
| | | | ATTCAAATTATATATATTTGGTCCAGTACGGTATCAATATATTATCAGTA |
| | | | (SEQ ID NO 39) |
| TMD0890 | TCACCACCACTGGGACC | GGCCACACCAATCACTGTGC | CAATCTGTTATTTATACGGCCTCTACATCCATCCAGTACCTGCTTATGTA |
| (SEQ ID NO | CTACAACCT | CAT | (SEQ ID NO 44) |
| 40-41) | (SEQ ID NO 42) | (SEQ ID NO 43) | GTTCTCTTTTTATAAAAGGCTATGTGGGACTTGCAAAACTTCTAGTGGCC |
| | | | (SEQ ID NO 45) |
| | | | GAACATGAAATATAAGTAGGGGAGTATCTTGGGGTAGAAAGGATGCCGAG |
| | | | (SEQ ID NO 46) |
| TMD1780 | CTCTGAAATCTTCTACAC | ATGAGATGGGAAGCACAGGT | ATCAATATTGTTAAAATGGCCGTACTGTCAAAAGCAATTTACAGATTCAA |
| (SEQ ID NO | AACTGTTATTCTGCCCA | GGAGAAG | (SEQ ID NO 51) |
| 47-48) | (SEQ ID NO 49) | (SEQ ID NO 50) | ATATGAAACCAAAAAAGCCCTCAAATAGCCCAAGTAACCCTAAAGAAAAA |
| | | | (SEQ ID NO 52) |
| | | | CGCCCTATTCAATAAATGGTGTGGGAATAGCTGGCTAGCCATCTGCAGAA |
| | | | (SEQ ID NO 53) |
| | | | CATAAGGGTTCTTAAAATTGGGAGAGAGAATCAGAAAGTCAGAGAAAGAG |
| | | | (SEQ ID NO 54) |
| TMD1781 | ATGACAGTTTATGATTCC | TCAGGATGGTGTGAACAATG | TTCCCTATTTAATAAATGGTGCTGGGAAAACTGGCTAGCCATATGTAGAA |
| (SEQ ID NO | TATGTTGCCATCTGC | AAGCCATAG | (SEQ ID NO 59) |
| 55-56) | (SEQ ID NO 57) | (SEQ ID NO 58) | AACAACCCCATCAAAAAGTGGGCCAAAGATATGAACAGACACTTCTCAAA |
| | | | (SEQ ID NO 60) |
| | | | AATGGCGATCATTAAAAAGTCAGGAAACAACAGGTGCTGGAGAGGATGTG |
| | | | (SEQ ID NO 61) |
| | | | CCCAGAGGATTATAAATCATGCTGCTGTAAAGACACATGCCCACGTATGT |
| | | | (SEQ ID NO 62) |

TABLE 6

| SEQ | | | PREDOMINANT | | PRIMER |
|---|---|---|---|---|---|
| ID | GENE | GENBANK | SITES OF | PROMOTER | (FOR, REV) |
| NO | NUMBER | IDENTIFIER | EXPRESSION | (SEQ ID NO) | (SEQ ID NO) |
| 63, 64 | TMD0785 | XM_060310 | kidney | 65-68 | 69, 70 |

TABLE 7

| | XM_062147 | XM_061676 |
|---|---|---|
| outside | 1-27 | 1-28 |
| TM (1) | 28-50 | 29-51 |
| inside | 51-61 | 52-62 |
| TM (2) | 62-84 | 63-85 |
| outside | 85-98 | 86-99 |
| TM (3) | 99-121 | 100-122 |
| inside | 122-140 | 123-133 |
| TM (4) | 141-163 | 134-156 |
| outside | 164-203 | 157-201 |
| TM (5) | 204-226 | 202-224 |
| inside | 227-237 | 225-236 |
| TM (6) | 238-260 | 237-259 |
| outside | 261-274 | 260-273 |
| TM (7) | 275-293 | 274-296 |
| inside | 294-313 | 297-314 |

TABLE 8

| | | | Predominant | |
|---|---|---|---|---|
| Clone ID | | | sites of | |
| (gene code) | ACCN | Gene Name/Description | expression | Other expression sites |
| TMD0049 | XM_057351 | Homo sapiens similar to organic anion transpoter 4 like protein | kidney | none |
| | | (LOC116085) mRNA | | |
| TMD0190 | XM_087157 | Homo sapiens similar to sodium-coupled ascorbic acid transporter 2 | kidney | colon and liver |
| | | (LOC151295), mRNA. | | |
| TMD0242 | XM_088369 | Homo sapiens similar to unnamed protein product (LOC157724) mRNA | kidney | none |
| TMD0335 | XM_089960 | Homo sapiens similar to sodium iodide symporter (LOC159963) mRNA | kidney | adrenal gland, heart, |
| | | | | intestine(small), liver, muscle, |
| | | | | testis |
| TMD0371 | XM_089732 | Homo sapiens similar to CG8271 gene product (LOC196023), mRNA. | kidney | pancreas and testis |
| (new) | | | | |
| TMD0374 | XM_085595 | Homo sapiens similar to unnamed protein product (LOC146802) mRNA | kidney | brain, muscle, ovary, skin, |
| (new) | | | | testis |
| TMD0469 | XM_038736 | Homo sapiens solute carrier family 4 sodium bicarbonate | kidney | none |
| | | cotransporter member 9 (SLC4A9) mRNA | | |
| TMD0719 | XM_059548 | Homo sapiens hypothetical gene supported by XM_059548 | kidney | none |
| | | (LOC131920) mRNA | | |
| TMD0731 | XM_059703 | Homo sapiens similar to putative (H. sapiens) (LOC134288) mRNA | kidney | adrenal gland, muscle, thyroid |
| TMD0785 | XM_060310 | Homo sapiens similar to olfactory receptor MOR2752 (LOC127069), | kidney | none |
| | | mRNA | | |
| TMD0841 | XM_060623 | Homo sapiens similar to KIAA0711 gene product (H. sapiens) | kidney | lung |
| | | (LOC127707) mRNA | | |
| TMD1114 | NM_019841 | Homo sapiens transient receptor potential cation channel | kidney | none |
| | | subfamily V member 5 (TRPV5) mRNA | | |
| TMD1148 | XM_087108 | Homo sapiens similar to calcium channel voltage-dependent | kidney | none |
| | | gamma subunit 6 (LOC151151) mRNA | | |

TABLE 9

| | SEQ | | |
|---|---|---|---|
| | ID | Protein seq | |
| Gene Code | NO | length (aa) | domain description |
| TMD0049 | 2 | 332 | Sugar (and other) transporter: 2-302 |
| | | | Transmembrane domain: 12-34 |
| | | | Transmembrane domain: 39-58 |
| | | | Transmembrane domain: 131-153 |
| | | | Transmembrane domain: 157-179 |
| | | | Transmembrane domain: 186-205 |
| | | | Transmembrane domain: 215-237 |
| TMD0190 | 4 | 243 | Permease family: 91-224 |
| TMD0242 | 6 | 470 | AA-permease: 27-356 |
| | | | Transmembrane domain: 13-35 |
| | | | Transmembrane domain: 50-72 |
| | | | Transmembrane domain: 93-115 |
| | | | Transmembrane domain: 137-154 |
| | | | Transmembrane domain: 161-183 |
| | | | Transmembrane domain: 207-229 |
| | | | Transmembrane domain: 242-264 |
| | | | Transmembrane domain: 286-308 |
| | | | Transmembrane domain: 335-357 |
| | | | Transmembrane domain: 362-379 |
| | | | Transmembrane domain: 392-414 |
| | | | Transmembrane domain: 420-442 |
| TMD0335 | 8 | 178 | Sodium solute symporter |
| | | | family: 41-172 |
| TMD0371 | 10 | 516 | Transmembrane domain: 45-67 |
| | | | Transmembrane domain: 87-109 |
| | | | Transmembrane domain: 116-138 |
| | | | Transmembrane domain: 143-165 |
| | | | Transmembrane domain: 174-196 |
| | | | Transmembrane domain: 201-223 |
| | | | Transmembrane domain: 283-305 |
| | | | Transmembrane domain: 320-339 |
| | | | Transmembrane domain: 351-370 |
| | | | Transmembrane domain: 375-397 |
| | | | Transmembrane domain: 404-426 |
| | | | Transmembrane domain: 441-463 |
| TMD0374 | 12 | 566 | Transmembrane domain: 31-53 |
| | | | Transmembrane domain: 68-90 |
| | | | Transmembrane domain: 116-138 |
| | | | Transmembrane domain: 153-171 |
| | | | Transmembrane domain: 184-206 |
| | | | Transmembrane domain: 211-233 |
| | | | Transmembrane domain: 254-273 |
| | | | Transmembrane domain: 288-310 |
| | | | Transmembrane domain: 331-353 |
| | | | Transmembrane domain: 373-395 |
| | | | Transmembrane domain: 404-426 |
| | | | Transmembrane domain: 431-453 |
| | | | Transmembrane domain: 542-564 |
| TMD0469 | 14 | 983 | HCO3-transporter family: 108-891 |
| | | | Transmembrane domain: 413-435 |
| | | | Transmembrane domain: 447-469 |
| | | | Transmembrane domain: 498-520 |
| | | | Transmembrane domain: 532-554 |
| | | | Transmembrane domain: 623-645 |
| | | | Transmembrane domain: 665-684 |
| | | | Transmembrane domain: 712-731 |
| | | | Transmembrane domain: 751-773 |
| | | | Transmembrane domain: 813-832 |
| | | | Transmembrane domain: 839-858 |
| | | | Transmembrane domain: 897-919 |
| TMD0719 | 16 | 146 | Transmembrane domain: 7-29 |
| | | | Transmembrane domain: 49-71 |
| TMD0731 | 18 | 218 | Transmembrane domain: 38-60 |
| | | | Transmembrane domain: 70-92 |
| TMD0785 | 20 | 312 | 7 transmembrane receptor |
| | | | (rhodopsin family): 58-290 |
| | | | Transmembrane domain: 29-51 |
| | | | Transmembrane domain: 61-83 |
| | | | Transmembrane domain: 140-162 |
| | | | Transmembrane domain: 197-219 |
| | | | Transmembrane domain: 240-262 |
| | | | Transmembrane domain: 272-294 |
| TMD0841 | 22 | 1161 | Kelch motif: 850-895 |
| | | | Kelch motif: 897-938 |
| TMD1114 | 24 | 729 | Transmembrane domain: 327-349 |
| | | | Transmembrane domain: 383-405 |
| | | | Transmembrane domain: 420-438 |
| | | | Transmembrane domain: 451-473 |
| | | | Transmembrane domain: 493-512 |
| | | | Transmembrane domain: 519-541 |
| | | | Transmembrane domain: 554-576 |
| TMD1148 | 26 | 103 | Transmembrane domain: 7-24 |
| | | | Transmembrane domain: 39-61 |
| | | | Transmembrane domain: 68-90 |

TABLE 10

| Clone ID (gene code) | ACCN | Cytogenetic locus | disease linkage |
|---|---|---|---|
| TMD0049 | XM_057351 | 11q12.1 | osteoporosis-pseudoglioma syndrome; spastic paraplega 1 7 |
| TMD0190 | XM_087157 | 2q36.2 | none |
| TMD0242 | XM_088369 | 8q21.2 | none |
| TMD0335 | XM_089960 | 11p14.2 | none |
| TMD0371A | XM_089732 | 10q23.33 | epilepsy, partial, with auditory features; spastic paraplegia 9, autosomal dominant |
| TMD0374 | XM_085595 | 17p11.2 | smith-magenis syndrome |
| TMD0469 | XM_038736 | 5q31 | paget disease of bone 4 |
| TMD0719 | XM_059548 | 3q29 | none |
| TMD0731 | XM_059703 | 5q13.2 | spastic paraplegia 11, autosomal recessive; |
| | | | corpus callosum, agenesis of, with neuronopathy |
| TMD0785 | XM_060310 | 1q44-tel | familial cold urticaria (FCU); Muckle-Wells syndrome (MWS); prostate cancer |
| | | | susceptibility |
| TMD0841 | XM_060623 | 1p36.13 | breast cancer, ductal, 2; |
| | | | prostate cancer/brain cancer susceptibility; |
| | | | melanoma, cutaneous |
| TMD1114 | NM_019841 | 7q35 | glaucoma 1, open angle, f |
| TMD1148 | XM_087108 | 2q14.1 | motor neuronopathy, distal hereditary, with vocal cord paralysis; cardiomyopathy, |
| | | | dilated, 1 h |

TABLE 11

| CODE (SEQ | | |
|---|---|---|
| ID NO | OLIGOS (SEQ ID NO) | PROMOTERS (SEQ ID NO) |
| TMD0049 | GCGCTTCCGGACCTGTATCTCCAC (104) | AAAGAGCCTCTAAAGAAGGGTTCCAGACTACCAGGAGCTCACTGGAAATA (106) |
| (78,79) | CAAGCTCTGGGTCTCGGGCAGAAG (105) | |
| TMD0190 | ACCATCCTGCAAACTTGGATGGGC (107) | GCTTTATGTATATGAAAACCCTGTTTATCTGAGCCTAGAACTGTCTTTGC (109) |
| (80,81) | AAGGAGCCGGAAGACAGGGAGAGG (108) | AGTGATAGTTTTAAATGGGAGGGAATAAAGTCTGCAAAATTTCCCCATAT (110) |
| TMD0242 | GAGTCTCCCTGTGCGTTTGGGCTG (111) | AGTCCCAGCTTAAAAAAGAGACAGACAGACAGAGAGAGAGAGAGACAGAG (113) |
| (82,83) | AAGTGTAAAGCATGCCCCGCCTGA (112) | TTAGTGATTTAAAAAAATGTGAAGAAGAGAGAGTCAAGGCAGTAAAAGGA (114) |
| TMD0335 | GTTCGCTATGCTGCCACGGTCATC (115) | GATACAAATAATTAAAAGCCCAGGTTAAGGTAAATATATTAAAGACCAAG (117) |
| (84,85) | AGTCCTGGCAGTCCTGGCATTGTG (116) | ATCTCACGAATTAAAAATGCTGAGGTGGTAAATTGTTATCAATTCTATGT (118) |
| TMD0371 | CAGGATTACCCACAAACGGCATGG (119) | CTAGACTATTTAAAAAAACCCCTGGCTTGCACAGTGGCTCAAGCCTGTAA (121) |
| (86,87) | TGGGAGGCAGAGATAGCAGAGCCC (120) | |
| TMD0374 | CTGGTCCTGGGCACCCTGATAAGC (122) | AGCTGTCCTCATTAAAAGTGACCTGGAGTGAGATGGATTCTTCTGCCTAT (124) |
| (88,89) | CCCAGGTCTGGTTGCAGTGCTCTC (123) | CCAATTCTTCTGAAAAACGGGAGTCACTGTGGGCACCATCACGCCCGGGT (125) |
| TMD0469 | CTGAGGTGTCCCTCCCAAGCAGGT (126) | TAAACAAATACATAAATGAGGCAGTTACTAGTAGTGGTAACTCCTAGGAA (128) |
| (90,91) | TACGGCCGAGAAGCACTGGAGATG (127) | ACTAAAAATATAAAAATCAGCCAGGCCTGGTGGCACATGTCTGTAATCTC (129) |
| | | GGGATGCATTATAAATGCAACCAGCCAGAGGGCCCCTGGCTTCAGAACCT (130) |
| TMD0719 | GTCACCTCAGCGATCTCAACGATAGGG (131) | ATATACCTTGTTTAAAAGAGGGGTATTATCACAATAAAACAAGGAAAGCT (133) |
| (92,93) | TGGAGCAGGAACAGGATATAGGTCAGGG (132) | ACCCCTACTTTTAAAGGCCTTGACAAACAGTGCTAAAGTTCTCACCTTAA (134) |
| TMD0731 | GGGTGGGAAGGAAGCAGGGAAGAG (135) | TTATTGGGCATAAAAATATGAAGAGAGGTCCCAGAGAGTCCCTAGGTTCT (137) |
| (94,95) | CCAGCTAGTTCATGCTTGGCGCAG (136) | |
| TMD0785 | CTGTTGGGAATCTTCAGCCAGATCTCACAC (138) | AAGCAATTTGTTAAAAACTGGCATTACTTTACTCTTATGCTTTCTGTGTC (140) |
| (96,97) | ATGGAGGTTTCTGCACGCTCAGCA (139) | ACTTTAATTTTATAAAGAAGGTTCACATCAAGAAATTCCAAGTGAGGTTC (141) |
| TMD0841 | GGGCCACTTCCACAGACAGGAAGC (142) | AAGGCTTCTTCAAAAAAAGCGGGCTTGTTCTGGGCCAGAAAATCAGAGTG (144) |
| (98,99) | TGGCCTGAGAGGTAGATTCCACATAGTAGTCGT | |
| | (143) | |
| TMD1114 | CTCCTTTCTGGTCACAGAACAAGACTGGGAC | CAGCGAGGCAGAAAAATGTCCCACAAGTTGAGCCCTCCCCACTCCCAGTG (147) |
| | (145) | |
| (100,101) | GTGATGTCTCGAGAATGAGTGCGGTTG (146) | TAATATAAAATATATAAAATAGTGCAACATTACTTATTCCTCCTGGTGTT (148) |
| TMD1148 | GCAGATGACCCGACCTGACTGTTCTTC (149) | GCCAGAGAGTTTAAATGAAGCCCTACTTTGGGGCAGGAGCGGGAGGAAAC (151) |
| (102,103) | TGGCTGTGCAGCTAGCTCAGGTACCAG (150) | |

TABLE 12

| SEQ | | | PREDOMINANT | OTHER | | PRIMER |
|---|---|---|---|---|---|---|
| ID | GENE | GENBANK | SITES | SITES OF | PROMOTER | (FOR, REV) |
| NO | NUMBER | IDENTIFIER | OF EXPRESSION | EXPRESSION | (SEQ ID NO) | SEQ ID NO |
| 152, | TMD0986 | XM_061779 | pancreas | low levels in | 156-161 | 154, 155 |
| 153 | | | | testis | | |
| 162, | TMD0987 | XM_061780 | pancreas | low levels in | 166 | 164, 165 |
| 163 | | | | testis | | |
| 167, | TMD0353 | XM_061781 | pancreas | | | 169, 170 |
| 168 | | | | | | |
| 171, | TMD0989 | XM_061784 | pancreas | | | 173, 174 |
| 172 | | | | | | |
| 175, | TMD058 | XM_061785 | pancreas | low levels in | 179, 180 | 177, 178 |
| 176 | | | | testis | | |

TABLE 13

| | XM_061779 | XM_061780 | XM_061781 | XM_061784 | XM_061785 |
|---|---|---|---|---|---|
| outside | 1-23 | 1-25 | 1-22 | | 1-24 |
| TM (1) | 24-46 | 26-48 | 23-45 | | 25-47 |
| inside | 47-58 | 49-60 | 46-65 | | 48-59 |
| TM (2) | 59-78 | 61-83 | 66-88 | | 60-82 |
| outside | 79-97 | 84-97 | 89-97 | | 83-96 |
| TM (3) | 98-120 | 98-120 | 98-120 | | 97-119 |
| inside | 121-140 | 121-139 | 121-140 | | 120-139 |
| TM (4) | 141-163 | 140-162 | 141-163 | | 140-162 |
| outside | 164-198 | 163-202 | 164-203 | | 163-201 |
| TM (5) | 199-221 | 203-25 | 204-226 | | 202-224 |
| inside | 222-240 | 226-237 | 227-237 | | 225-236 |
| TM (6) | 241-260 | 238-260 | 238-260 | | 237-259 |
| outside | 261-274 | 261-269 | 261-272 | | 260-268 |
| TM (7) | 75-292 | 270-289 | 273-292 | | 269-291 |
| inside | 293-314 | 290-318 | 293-323 | | 292-311 |

TABLE 14

| GENBANK | MOUSE | | | | | |
|---|---|---|---|---|---|---|
| IDENTIFIER | HOMOLOG | 061779 | 061780 | 061781 | 061784 | 061785 |
| XM_061779 | | | 42% (63%) | 36% (57%) | 43% (64%) | 40% (61%) |
| XM_061780 | MOR239-6 | 42% (63%) | | 41% (60%) | 44% (62%) | 46% (67%) |
| | (AY073489) | | | | | |
| | 90% (93%) | | | | | |
| XM_061781 | | 36% (57%) | 41% (60%) | | 43% (63%) | 40% (61%) |
| XM_061784 | MOR223 | 43% (64%) | 44% (62%) | 43% (63%) | | 81% (87%) |
| | ˜>85% | | | | | |
| XM_061785 | MOR223 | 40% (61%) | 46% (67%) | 40% (61%) | 81% (87%) | |
| | ˜>85% | | | | | |

TABLE 15

| | | Predominant | Other | |
|---|---|---|---|---|
| Clone ID | | sites of | expression | Cytogenetic |
| (gene code) | ACCN | expression | sites | locus |
| TMD1030 (SEQ | XM_166853 | spleen | liver | 11q12.2 |
| ID NO 185-186) | | | | |
| TMD1029 (SEQ | XM_166854 | spleen, | brain, | 11q12.2 |
| ID NO 187-188) | | lymphocytes, | heart, | |
| | | liver | lung, | |
| | | | lymph | |
| | | | node | |
| TMD1028 (SEQ | XM_166855 | spleen, | liver | 11q12.2 |
| ID NO 189-190) | | lymphocytes | | |
| TMD0621 (SEQ | XM_166205 | spleen | brain, | 11q12.2 |
| ID NO 191-192) | | | heart, | |
| | | | liver, | |
| | | | lung | |
| | | | and | |
| | | | pancreas | |

TABLE 16

| | | Protein | |
|---|---|---|---|
| Clone ID | ACCN | length (aa) | domain description |
| TMD1030 | XM_166853 | 298 | Transmembrane domain: 27-49 |
| | | | Transmembrane domain: 98-120 |
| | | | Transmembrane domain: 140-162 |
| | | | Transmembrane domain: 175-197 |
| | | | Transmembrane domain: 207-226 |
| | | | Transmembrane domain: 238-260 |
| | | | Transmembrane domain: 275-292 |
| TMD1029 | XM_16684 | 309 | Transmembrane domain: 26-48 |
| | | | Transmembrane domain: 61-78 |
| | | | Transmembrane domain: 98-120 |
| | | | Transmembrane domain: 140-162 |
| | | | Transmembrane domain: 196-218 |
| | | | Transmembrane domain: 238-260 |
| | | | Transmembrane domain: 275-292 |
| TMD1028 | XM_166855 | 173 | Transmembrane domain: 18-40 |
| | | | Transmembrane domain: 61-83 |
| | | | Transmembrane domain: 103-125 |
| | | | Transmembrane domain: 137-156 |
| TMD0621 | XM_166205 | 109 | Transmembrane domain: 9-31 |
| | | | Transmembrane domain: 69-91 |

TABLE 17

| | | | |
|---|---|---|---|
| Clone ID | ACCN | F-oligo | R-oligo |

| | | | |
|---|---|---|---|
| TMD1030 | XM_166853 | GGGATTTGGTGTCCAACACGAATTTCA | GAGCCTATAATATATGAGCCAGCTACGAGTTGGA |
| | | (SEQ ID NO 197) | (SEQ ID NO 198) |
| TMD1029 | XM_166854 | GTCACTGAATTCTATCTTCTGGGATTTGGTGC | AAACCTGTTTGTACAGAGGCATTTATTGAGCC |
| | | (SEQ ID NO 199) | (SEQ ID NO 200) |
| TMD1028 | XM_166855 | GATATCATTTTGGGGCTGCATGATACAATTATTGG | CTCCAACCCAGTGAACATCAAGTTAAATCCCAC |
| | | (SEQ ID NO 201) | (SEQ ID NO 202) |
| TMD0621 | XM_166205 | TTAAGCTATTAGTTAGTTCATATGTCATGGGTTTCC | CTCATTAATACGATGGCATAGATACATGTAAGAGAG |
| | | (SEQ ID NO 203) | (SEQ ID NO 204) |

TABLE 18

| | | |
|---|---|---|
| Clone ID | ACCN | Promoter Sequence (likelihood score) |

| | | |
|---|---|---|
| TMD1030 | XM_166853 | ATGTTCCATCTAAATGAAGCCTGAGAAACCCAGCACTACCCACTTGTTAG (0.94) (SEQ ID NO 205) |
| | | ACATCCATTATATAACAGGGTTAATATACTTGTAAAGAATAGCACCTAGA (0.95) (SEQ ID NO 206) |
| TMD1029 | XM_166854 | AAATGTATAAATTCTGCATGAAATTGGGGGTGGGGCTTGTACTACTTTTG (0.98) (SEQ ID NO 207) |
| TMD1028 | XM_166855 | ATGTTCCATCTAAATGAAGCCTGAGAAACCCAGCACTACCCACTTGTTAG (0.94) (SEQ ID NO 208) |
| | | ACATCCATTATATAACAGGGTTAATATACTTGTAAAGAATAGCACCTAGA (0.95) (SEQ ID NO 209) |
| TMD0621 | XM_166205 | AAATATATATTTTAAATTGGCCAGGCGCGGTGGCTCACGCCTATAATCCC (0.99) (SEQ ID NO 210) |
| | | GGCTCACGCCTATAATCCCAGCACTTTGGGAGGCCGAGGCAGGTGGATCA (0.97) (SEQ ID NO 211) |
| | | TCCCAAATATATATATATACACACACACACACACACACACACATATATAT (1.00) (SEQ ID NO 212) |
| | | CACACACACATATATATACACACACATATATTTATAATCATTTAACAACA (0.91) (SEQ ID NO 213) |

### TABLE 19

(from Principles of Internal Medicine, Volume 1, Page 357, 12^{th} Edition, McGraw-Hill Inc.)

TABLE 20

| | | | Predominant sites of | |
|---|---|---|---|---|
| Clone ID | ACCN | Gene Name/Description | expression | Other expression sites |
| TMD0077 | XM_166914 | Homo sapiens olfactory receptor MOR2121 (LOC219956), mRNA. | pancreas and testis | brain, heart and kidney |
| TMD0233 | XM_069616 | Homo sapiens similar to olfactory receptor (LOC135941) mRNA | pancreas | none |
| TMD0256 | XM_066725 | Homo sapiens similar to olfactory receptor (LOC139478) mRNA | pancreas | skin and testis |
| TMD0258 | XM_066873 | Homo sapiens similar to beta-2 adrenergic receptor (LOC139760) mRNA | pancreas | colon, stomach and testis |
| TMD0267 | XM_089550 | Homo sapiens similar to CG5281 gene product (LOC159371) mRNA | pancreas and testis | adrenal gland, bone |
| | | | | marrow, colon, heart, |
| | | | | intestine(small), kidney, |
| | | | | liver, pituitary, prostate, |
| | | | | skin, stomach and thyroid |
| TMD0271 | XM_061815 | Homo sapiens similar to odorant receptor S18 gene (LOC120010) mRNA | pancreas and testis | PBL, prostate, thymus |
| | | | | and uterus |
| TMD0290 | XM_065813 | Homo sapiens similar to unnamed protein product (LOC130644) mRNA | pancreas and testis | none |
| TMD0530 | XM_048304 | Homo sapiens hypothetical protein DKFZp564A1164 (DKFZP564A1164) | pancreas | brain, kidney, lung, lymph |
| | | mRNA | | node, PBL, mammary |
| | | | | gland, pituitary |
| | | | | stomach, testis and thyroid |
| TMD0574 | XM_055514 | Homo sapiens KIAA1910 protein (KIAA1910) mRNA | brain and pancreas | pituitary |
| TMD0608 | XM_058332 | Homo sapiens similar to putative (H. sapiens) (LOC118670) mRNA | pancreas and testis | stomach |
| TMD0639 | XM_058690 | Homo sapiens similar to data source: MGD, source key: MGI: 96073, | pancreas and testis | liver, PBL and prostate |
| | | evidence: ISS˜hexosaminidase A˜putative (LOC204249, mRNA. | | |
| TMD0645 | XM_085376 | Homo sapiens LOC146225 (LOC146225), mRNA. | pancreas and testis | bone marrow, brain, heart, |
| | | | | kidney, liver, lung, |
| | | | | lymph node, PBL, muscle, |
| | | | | pituitary, prostate, skin, |
| | | | | spleen, stomach and thymus |
| TMD0674 | XM_059132 | Homo sapiens similar to RIKEN cDNA 4930549C01 gene (LOC127309) | pancreas and testis | brain, pituitary, prostate |
| | | mRNA | | and stomach |
| TMD0675 | XM_059134 | Homo sapiens similar to putative (H. sapiens) (LOC127348) mRNA | pancreas and testis | prostate and stomach |
| TMD0677 | XM_059140 | Homo sapiens similar to dJ39G22.2 (novel protein) (H. sapiens) | pancreas and testis | prostate and stomach |
| | | (LOC127391) mRNA | | |
| TMD0726 | XM_059639 | Homo sapiens similar to hypothetical protein (H. sapiens) (LOC133309) | pancreas and testis | adrenal gland, brain, |
| | | mRNA | | prostate and stomach |
| TMD0727 | XM_059654 | Homo sapiens similar to testis-specific transporter TST1 (H. sapiens) | pancreas and testis | stomach |
| | | (LOC133482) mRNA | | |
| TMD0739 | XM_059812 | Homo sapiens similar to putative (H. sapiens) (LOC135886) mRNA | pancreas and testis | liver, lung, mammary |
| | | | | gland, ovary, pituitary, |
| | | | | prostate and stomach |
| TMD0753 | XM_059954 | Homo sapiens similar to putative (H. sapiens) (LOC138240) mRNA | pancreas and testis | none |
| TMD1111 | NM_014386 | Homo sapiens polycystic kidney disease 2-like 2 (PKD2L2) mRNA | pancreas and testis | none |
| TMD1127 | NM_054020 | Homo sapiens putative ion channel protein CATSPER2 (CATSPER2), | pancreas and testis | none |
| | | mRNA. | | |

TABLE 21

| | | Protein seq | |
|---|---|---|---|
| Clone ID | ACCN | length (aa) | Domain description |
| TMD0077 | XM_166914 | 310 | 7 transmembrane receptor (rhodopsin family) |
| | | | Transmembrane domains: 27-49 |
| | | | Transmembrane domains: 61-83 |
| | | | Transmembrane domains: 98-120 |
| | | | Transmembrane domains: 141-163 |
| | | | Transmembrane domains: 202-224 |
| | | | Transmembrane domains: 237-259 |
| | | | Transmembrane domains: 274-291 |
| TMD0233 | XM_069616 | 310 | 7 transmembrane receptor (rhodopsin family) |
| | | | Transmembrane domain: 26-48 |
| | | | Transmembrane domain: 60-77 |
| | | | Transmembrane domain: 97-119 |
| | | | Transmembrane domain: 140-162 |
| | | | Transmembrane domain: 196-218 |
| | | | Transmembrane domain: 239-261 |
| | | | Transmembrane domain: 272-291 |
| TMD0256 | XM_066725 | 308 | 7 transmembrane receptor (rhodopsin family) |
| | | | Transmembrane domain: 27-49 |
| | | | Transmembrane domain: 61-83 |
| | | | Transmembrane domain: 98-120 |
| | | | Transmembrane domain: 140-162 |
| | | | Transmembrane domain: 196-218 |
| | | | Transmembrane domain: 239-258 |
| | | | Transmembrane domain: 273-291 |
| TMD0258 | XM_066873 | 335 | 7 transmembrane receptor (rhodopsin family) |
| | | | Transmembrane domain: 10-32 |
| | | | Transmembrane domain: 39-61 |
| | | | Transmembrane domain: 79-101 |
| | | | Transmembrane domain: 121-143 |
| | | | Transmembrane domain: 163-185 |
| | | | Transmembrane domain: 226-248 |
| | | | Transmembrane domain: 263-282 |
| TMD0267 | XM_089550 | 324 | Integral membrane protein DUF6: 49-161 |
| | | | Transmembrane domain: 59-78 |
| | | | Transmembrane domain: 91-110 |
| | | | Transmembrane domain: 115-137 |
| | | | Transmembrane domain: 146-168 |
| | | | Transmembrane domain: 183-201 |
| | | | Transmembrane domain: 214-236 |
| | | | Transmembrane domain: 246-265 |
| | | | Transmembrane domain: 270-292 |
| | | | Transmembrane domain: 297-316 |
| TMD0271 | XM_061815 | 291 | 7 transmembrane receptor (rhodopsin family) |
| | | | Transmembrane domain: 29-51 |
| | | | Transmembrane domain: 56-78 |
| | | | Transmembrane domain: 83-105 |
| | | | Transmembrane domain: 120-142 |
| | | | Transmembrane domain: 163-185 |
| | | | Transmembrane domain: 190-207 |
| | | | Transmembrane domain: 220-239 |
| | | | Transmembrane domain: 249-271 |
| TMD0290 | XM_065813 | 245 | Transmembrane domain: 24-46 |
| | | | Transmembrane domain: 61-83 |
| | | | Transmembrane domain: 96-118 |
| | | | Transmembrane domain: 128-150 |
| | | | Transmembrane domain: 162-184 |
| | | | Transmembrane domain: 221-243 |
| TMD0530 | XM_048304 | 708 | Immunoglobulin domain: 139-206 |
| | | | Immunoglobulin domain: 326-377 |
| | | | Transmembrane domain: 511-533 |
| TMD0574 | XM_055514 | 696 | Leucine rich repeat C-terminal domain: 212-262 |
| | | | Leucine rich repeat C-terminal domain: 529-579 |
| | | | Transmembrane domain: 621-643 |
| TMD0608 | XM_058332 | 105 | Transmembrane domain: 13-35 |
| TMD0639 | XM_058690 | 127 | Transmembrane domain: 12-34 |
| | | | Transmembrane domain: 44-66 |
| TMD0645 | XM_085376 | 248 | Transmembrane domain: 113-135 |
| | | | Transmembrane domain: 150-169 |
| | | | Transmembrane domain: 176-198 |
| TMD0674 | XM_059132 | 134 | Transmembrane domain: 5-22 |
| TMD0675 | XM_059134 | 206 | Transmembrane domain: 15-37 |
| TMD0677 | XM_059140 | 182 | Transmembrane: 49-71 |
| TMD0726 | XM_059639 | 96 | Transmembrane domain: 13-35 |
| | | | Transmembrane domain: 50-72 |
| TMD0727 | related to | 719 | Transmembrane domain: 108-130 |
| | XM_059654 | | |
| | | | Transmembrane domain: 145-164 |
| | | | Transmembrane domain: 171-193 |
| | | | Transmembrane domain: 229-251 |
| | | | Transmembrane domain: 264-286 |
| | | | Transmembrane domain: 314-336 |
| | | | Transmembrane domain: 421-443 |
| | | | Transmembrane domain: 453-475 |
| | | | Transmembrane domain: 580-602 |
| | | | Transmembrane domain: 668-690 |
| | | | Organic Anion Transporter Polypeptide (OATP) family, C- |
| | | | terminus: 125-473 |
| | | | Organic Anion Transporter Polypeptide (OATP) family, N- |
| | | | terminus: 558-717 |
| TMD0739 | XM_059812 | 265 | Transmembrane domain: 126-148 |
| | | | Transmembrane domain: 185-207 |
| TMD0753 | XM_059954 | 161 | Transmembrane domain: 26-48 |
| TMD1111 | NM_014386 | 609 | Ion transporter domain: 284-490 |
| | | | Transmembrane domain: 34-56 |
| | | | Transmembrane domain: 274-296 |
| | | | Transmembrane domain: 315-337 |
| | | | Transmembrane domain: 364-386 |
| | | | Transmembrane domain: 407-429 |
| | | | Transmembrane domain: 469-491 |
| TMD1127 | NM_054020 | 528 | Ion transporter domain: 172-340 |
| | | | Transmembrane domain: 113-132 |
| | | | Transmembrane domain: 147-169 |
| | | | Transmembrane domain: 176-198 |
| | | | Transmembrane domain: 241-263 |
| | | | Transmembrane domain: 276-295 |
| | | | Transmembrane domain: 315-337 |

TABLE 22

| Clone ID | ACCN | Cytogenetic locus | disease linkage |
|---|---|---|---|
| TMD0077 | XM_166914 | 11q12.2 | angloedema, hereditary; spastic paraplegia 17; osteoporosis |
| | | | pseudoglioma syndrome; pancreatic tumor |
| TMD0233 | XM_069616 | 7q35 | glaucoma 1, open angle, f; |
| TMD0256 | XM_066725 | Xq26.1 | x inactivation, familial skewed, 2; panhypopituitarism; |
| | | | thoracoabdominal syndrome; dandy-walker malformation |
| | | | with mental retardation, basal ganglia disease, and seizures; |
| | | | split-hand/foot malformation 2; mental retardation with optic |
| | | | atrophy, deafness |
| TMD0258 | XM_066873 | Xq26.1 | x inactivation, familial skewed, 2; panhypopituitarism; |
| | | | thoracoabdominal syndrome; dandy-walker malformation |
| | | | with mental retardation, basal ganglia disease, and seizures; |
| | | | split-hand/foot malformation 2; mental retardation with optic |
| | | | atrophy, deafness |
| TMD0267 | XM_089550 | 10q24.1 | corneal dystrophy of bowman layer, type ii; alzheimer |
| | | | disease 6 |
| TMD0271 | XM_061815 | 11p15.4 | charcot-marie-tooth disease, type 4b, form 2; deafness, |
| | | | neurosensory, autosomal recessive 18; |
| TMD0290 | XM_065813 | 2p23.1 | none |
| TMD0530 | XM_048304 | 19q13.13 | hypocalciuric hypercalcemia, familial, type iii; |
| | | | deafness, autosomal dominant nonsyndromic sensorineural 4; |
| | | | microcephaly, primary autosomal recessive, 2 |
| TMD0574 | XM_055514 | 13q31.1 | microcoria, congenital; schizophrenia 7; |
| TMD0608 | XM_058332 | 10q26.3 | endometrial carcinoma |
| TMD0639 | XM_058690 | 15q22.32 | cataract, central saccular, with sutural opacities; obesity |
| | | | syndrome |
| TMD0645 | XM_085376 | 16q23.1 | dehydrated hereditary stomatocytosis; pancreatic acinar |
| | | | cancer |
| TMD0674 | XM_059132 | 1p36.11 | breast cancer, ductal, 2; |
| | | | prostate cancer/brain cancer susceptibility; melanoma, |
| | | | cutaneous malignant; |
| | | | inflammatory bowel disease 7; |
| TMD0675 | XM_059134 | 1p33 | carcinoma of pancreas |
| TMD0677 | XM_059140 | 1p34.2 | deafness, autosomal dominant nonsyndromic sensorineural |
| | | | 2; |
| | | | porphyria cutanea tarda; |
| | | | hypercholesterolemia, familial, ptosis, hereditary congenital 1; |
| TMD0726 | XM_059639 | 10q11.22 | none |
| TMD0727 | related to | 5q21.1 | anemia, dyserythropoietic congenital, type iii; |
| | XM_059654 | | dyslexia, specific, 1; |
| | | | colorectal cancer, hereditary nonpolyposis, type 7; |
| | | | cataract, central saccular, with sutural opacities |
| TMD0739 | XM_059812 | 7q11.23 | autism, susceptibility to, 1; |
| | | | muscular dystrophy, limb-girdle, type 1d; |
| | | | aneurysm, intracrania I |
| TMD0753 | XM_059954 | 9q21.12 | hemophagocytic lymphohistiocytosis, familial, 1; |
| | | | amyotrophic lateral sclerosis with frontotemporal dementia |
| TMD1111 | NM_014386 | 5q31 | none |
| TMD1127 | NM_054020 | 15q13-q15 | nanophthalmos 2; spastic paraplegia 11, autosomal |
| | | | recessive; corpus callosum, agenesis of, with neuronopathy; |
| | | | pancreatic acinar carcinoma |

TABLE 23

| | | | |
|---|---|---|---|
| CODE | ACCN | PRIMERS | PROMOTER |

| | | | |
|---|---|---|---|
| TMD0077 | XM_166914 | TCATGGATCACCAGCTCCACGCTC | GGATTCAGGCCTTTTAAACCCCACTCAGTGGGTGCATGGCAGGGCTTTGA |
| (SEQ ID NO | | (Forward) (SEQ ID NO 256) | (0.88) (SEQ ID NO 258) |
| 214-215) | | CACCAAGATCACCACCATGGAAGCA | |
| | | (Reverse) (SEQ ID NO 257) | |
| TMD0233 | XM_069616 | TGCTGACGAATCTTATGAACCAGG | TCACAAATCATATAAATTAGGGGAAAGAGAGAGGCAGGTATACTCTAAAA |
| (SEQ ID NO | | (Forward) (SEQ ID NO 259) | (0.96) (SEQ ID NO 261) |
| 216-217) | | TCACGTCAGCCTCTCCTI7CCTCAGTG | AATTTCTTATTTAAAAGACCTCAGAAATGTCACCATGCTTAGTTATTTTA |
| | | (Reverse) (SEQ ID NO 260) | (0.95) (SEQ ID NO 262) |
| TMD0256 | XM_066725 | GGCCATGGACAATGTCACAGCAG | GGTACTATTCTATATTTTGGGCACACAGCAATGAAGAAAACAGAAAAACC |
| (SEQ ID NO | | (Forward) (SEQ ID NO 263) | (0.93) (SEQ ID NO 265) |
| 218-219) | | AGCAGACACATACTGGGCCATTCATAACC | CTGGGTTTCATAAATATGGAGCAGAAAGTTTTTACAAATATAGAACAGCA |
| | | AC (Reverse) (SEQ ID NO 264) | (0.92) (SEQ ID NO 266) |
| | | | TAGAATGTGTTATAAAAAATGAACCAGGGCTAGGGGAAAGAGATGGGTGA |
| | | | (0.91) (SEQ ID NO 267) |
| TMD0258 | XM_066873 | CCTCATTGGCTTCCTCCCACTCG | CCAAGGAACTTTTAAAACTCCCATTGCACAGTTACCACCCAGAATAATTA |
| (SEQ ID NO | | (Forward) (SEQ ID NO 268) | (97) (SEQ ID NO 270) |
| 220-221) | | GCCATCAAACTCTGAGCTGGAGATAGTGAC | CATCCTGGAATATATTTGCGTCCAAGTCTGCACCTTGCTCTCTATTCGCT |
| | | (Reverse) (SEQ ID NO 269) | (0.96) (SEQ ID NO 271) |
| | | | CTGGGGCCCCTGAAAAAGCTCACCTTCCCTCACTTCCCACTTCAACTGAT |
| | | | (0.91) (SEQ ID NO 272) |
| TMD0267 | XM_089550 | TGGCCTCGTTGAAAGTGTCATCATCC | AAACGGCATTTTAAAAATGCAGGTTTAAATTGTTATCCTCATCTATGGTT |
| (SEQ ID NO | | (Forward) (SEQ ID NO 273) | (0.98) (SEQ ID NO 275) |
| 222-223) | | TTGGTACCATTTACGAATGGCCGC | |
| | | (Reverse) (SEQ ID NO 274) | |
| TMD0271 | XM_061815 | CTGGACTTGAGCAGTACCACGTCTGGATC | ATTTTGGTTATATATAGAGGAGTCTAGGAAAAGACTCGTGGGTCTGATTC |
| (SEQ ID NO | | (Forward) (SEQ ID NO 276) | (0.97) (SEQ ID NO 278) |
| 224-225) | | CATATTCCCACAGCAATTTTGACAATGG | TACTCATATTTATATAGCAGCAACTTACATTGACCCAGGGAGAACTCAGT |
| | | (Reverse) (SEQ ID NO 277) | (0.94) (SEQ ID NO 279) |
| TMD0290 | XM_065813 | GTTACCCACCCAACCGTCACGACC | CTAGAATTTACATAAAAAGGACTGGAGGAGCTTTTGCAGCAACTTTGCAT |
| (SEQ ID NO | | (Forward) (SEQ ID NO 280) | (0.97) (SEQ ID NO 282) |
| 226-227) | | CAGGCGATGCCAGAGAAGACGATG | TTTTCTTCTTTTAAAAACACGCTTTCACTCTCAAAACAGCAGAGAATGAA |
| | | (Reverse) (SEQ ID NO 281) | (0.98) (SEQ ID NO 283) |
| | | | AACTGGGGTCTATAAGAGAGGCAGGGCACTTATTCATCCAAGGGCAGATG |
| | | | (0.99) (SEQ ID NO 284) |
| TMD0530 | XM_048304 | CTATGACTTCAACCCACACCTGGGCA | GGGCGGGAGTAAAAGGCAGAGTCCAATTCCACCGGCCCCCAGTGTGGGTG |
| (SEQ ID NO | | (Forward) (SEQ ID NO 285) | (0.86) (SEQ ID NO 287) |
| 228-229) | | AAGGTCGCCAACTTGTCCTGGCTC | |
| | | (Reverse) (SEQ ID NO 286) | |
| TMD0574 | XM_055514 | TCAATGCCATGCCCAAACTGAGGA | CTTTTAAGGTTAAAAATGTGGGTTTTAGATGATTGTCCTTTCTAAACAGC |
| (SEQ ID NO | | (Forward) (SEQ ID NO 288) | (0.99) (SEQ ID NO 290) |
| 230-231) | | CAACACCGAGATGGACACCCTGCT | TCAGGATGTCTAAAAAAGATCTCTCTAGTGTACACACGTGCACACACACA |
| | | (Reverse) (SEQ ID NO 289) | (0.97) (SEQ ID NO 291) |
| | | | AGTAACTCTATTTAAAAGACCTAAAAATTTCAAATCCTAAAATGATCTAT |
| | | | (0.90) (SEQ ID NO 292) |
| | | | AATAAATGTTTTAAAAGCACTCCTTTCCGAATGGTGGAGCTGGTGGGGGC |
| | | | (0.91) (SEQ ID NO 293) |
| TMD0608 | XM_058332 | CTCAGGACGAAGATCATGATCGGCATC | TATTCTCACTTATAAGTGGGAGCTAAGCCATGAGGGCACCAAGGCATAAG |
| (SEQ ID NO | | (Forward) (SEQ ID NO 294) | (0.99) (SEQ ID NO 296) |
| 232-233) | | GAAGATTTTTGTGCCCAGCTTTCCCAAG | TTACATATGTATACATGTGCCATGCTGGTGTGCTGCACCCATTAACTCGT |
| | | (Reverse) (SEQ ID NO 295) | (0.96) (SEQ ID NO 297) |
| TMD0639 | XM_058690 | TCCATGCTCAGCTTCATCTCAGCTACC | AAATAACCCCATTAAAAAGTGGGCAAAGGGCATGAACACTTTCAAAAGA |
| (SEQ ID NO | | (Forward) (SEQ ID NO 298) | (1.00) (SEQ ID NO 300) |
| 234-235) | | TCCATCTCAGACCTTGGCCCTTCA | |
| | | (Reverse) (SEQ ID NO 299) | |
| TMD0645 | XM_085376 | AGGACGGTAAGGAGCCATCGGACA | TCTTTTTGTCTATAAATAGGACTTTGATTTTCTGGACTACAGAATTGTAT |
| (SEQ ID NO | | (Forward) (SEQ ID NO 301) | (0.94) (SEQ ID NO 303) |
| 236-237) | | CTTGCCAGGTTCTGGTGGCTTGG | |
| | | (Reverse) (SEQ ID NO 302) | |
| TMD0674 | XM_059132 | ACGACTCCAAGAACAGCAAGGCCG | GCTAGCATTTTTTAAAAGCTGATGTCTTCACTGGGCACGGGACTCACAC |
| (SEQ ID NO | | (Forward) (SEQ ID NO 304) | (0.94) (SEQ ID NO 306) |
| 238-239) | | AAGGTAACATCGGCAGAGGCCAGC | |
| | | (Reverse) SEQ ID NO 305) | |
| TMD0675 | XM_059134 | CGGCCAGGTACCAAAGCTCAGCTG | TGATCTACTTTTTAAAAGGATCATGCTGGCTGCTGGTGGCATTTAGGATA |
| (SEQ ID NO | | (Forward) (SEQ ID NO 307) | (0.91) (SEQ ID NO 309) |
| 240-241) | | GCCAGATTCAGGAGGGAATGGAAGAGAAC | TGATAGTGATAAAAAAAAGTGGCCAGATTTTGGTTATATTTGAAATAAA |
| | | (Reverse) (SEQ ID NO 308) | (0.99) (SEQ ID NO 310) |
| | | | TATAGTGATATTTAAAGCCAGGGGTCTGGGTGAGATAACTGATGGAATGA |
| | | | (0.93) (SEQ ID NO 311) |
| | | | ATTGGAGGACTATAAAGAGGGGAGTCATTAAAATGGTGCTAAGAAGCTGA |
| | | | (0.96) (SEQ ID NO 312) |
| | | | AGAGGGGAGTCATTAAAATGGTGCTAAGAAGCTGAGCTACAAGCAGTGGT |
| | | | (0.97) (SEQ ID NO 313) |
| | | | GACATTCCACCCAAAAAATGCCACTGGATGAAGTCCCCTCTTCCATTAA |
| | | | (0.92) (SEQ ID NO 314) |
| TMD0677 | XM_059140 | TTGGGAGAGACTAGTGCACCTCAGCA | AAAAGTGCTTTTAAACAGGGGGGGTGGAGGGGCTTATGACAAGGGGACCA |
| (SEQ ID NO | | (Forward) (SEQ ID NO 315) | (1.00) (SEQ ID NO 317) |
| 242-243) | | GAGCAATCCCTCTITCGTGGCAGGT | CCATTTCTACTAAAAATGCAGAGATCAGCCAGGCGTGCCACGTGCCTGTA |
| | | (Reverse) (SEQ ID NO 316) | (0.95) (SEQ ID NO 318) |
| | | | AAAAAAAAAAAAAAAAAGCCCTGTTTATATCCTACCTCCTGCTGGGTGC |
| | | | (0.98) (SEQ ID NO 319) |
| | | | AAAATAAAAATAAAAAATCCCATCTCCTCACATTTCCATTCAACCTCAAT |
| | | | (0.93) (SEQ ID NO 320) |
| TMD0726 | XM_059639 | ACTTCCAAACATCTACAACTCCTCAGAGT | TTTTTTAAACTATAAAAAGTGGGGATCAGAAAACACAGTCATAAGGGAAA |
| (SEQ ID NO | | CTCATT (Forward) | (0.97) (SEQ ID NO 323) |
| 244-245) | | (SEQ ID NO 321) | GTATATGCTATATATATCAGGATTCACTTTAATGGCATTCAGTTCCAGGA |
| | | TGCAGCACCATCATGTAAGGGACAA | (0.98) (SEQ ID NO 324) |
| | | (Reverse) (SEQ ID NO 322) | ATAAAGAATTTAAAAATTAGCCCACCATGGTGGTACACACTGTCGTTCT |
| | | | (0.99) (SEQ ID NO 325) |
| | | | AAAAAGTGAAAAAAAAAGGTGAGGGAGACTTTAACTTTCTGAAATATATT |
| | | | (0.92) (SEQ ID NO 326) |
| TMD0727 | XM_059654 | CCAAGAAGCCGGGAGAAGTGGATG | CTAAAGAGCTTATATATCAGCCTAAGAAAAGAAAACCAATAAGAAGTTGC |
| (SEQ ID NO | (related to) | (Forward) (SEQ ID NO 327) | (0.96) (SEQ ID NO 329) |
| 246-247) | | TGACAGAGCTAGGCATATGAGCACTGGA | |
| | | (Reverse) (SEQ ID NO 328) | |
| TMD0739 | XM_059812 | GCAGTTGGTTGAGAACCGAGATCACC | ACTAAAAATACAAAAAAGTAGCCGGGTATGGTGGTAGCGCCTATAATCC |
| (SEQ ID NO | | (Forward) (SEQ ID NO 330) | (0.93) (SEQ ID NO 332) |
| 248-249) | | GGCAGATGGGGATACATTTATTCTCTGGG | GGTAGGCGCCTATAATCCCAGCTACTTGGGAGGCTGAGGGAGGAGAATTG |
| | | (Reverse) (SEQ ID NO 331) | (0.92) (SEQ ID NO 333) |
| TMD0753 | XM_059954 | TCGGCTTGGAAATCAGAATGAGAAGG | AAAAGGCTTATATAAAAGGGTTTTGTTTTGTTTTGTTTTCAGACGGAGTT |
| (SEQ ID NO | | (Forward) (SEQ ID NO 334) | (0.97) (SEQ ID NO 336) |
| 250-251) | | TGCACAAAGAATGATTGCAGCAGTGAGTAG | GGCCAACTTATATAAAAGGTTTATGTTTTTGTTCTGATAATTTCGTTTCT |
| | | (Reverse) (SEQ ID NO 335) | (0.91) (SEQ ID NO 337) |
| | | | AAGTTAAGTTTTAAAAAGAACAGGCTACAAAGTTATAGCTATGGGGTGAT |
| | | | (0.96) (SEQ ID NO 338) |
| TMD1111 | NM_014386 | GGGCGGTGTAGTGGAGGTCCG | AATTCAAATATTTAAAACGGACTGTCTCCTCTTCACAAAAGTCTAGATCT |
| (SEQ ID NO | | (Forward) (SEQ ID NO 339) | (0.92) (SEQ ID NO 341) |
| 252-253) | | CCTCCAGTTGCAGGGAATTCTGCC | |
| | | (Reverse) (SEQ ID NO 340) | |
| TMD1127 | NM_054020 | GGCTGTTGAGCAGGCTTCATGTGC | ATTGGGTGCATATATATTTAGGATAGTTAGCTCTTCTTGTGAATTGATC |
| (SEQ ID NO | | (Forward) (SEQ ID NO 342) | (0.89) (SEQ ID NO 344) |
| 254-255) | | CTCCTCTGGATGATCTGCCGCTTG | |
| | | (Reverse) (SEQ ID NO 343) | |

1-171. (canceled)

172. A method of detecting a pancreas cell, comprising:
contacting a sample comprising cells with a polynucleotide specific TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127 of claim 199 under conditions effective for said polynucleotide to hybridize specifically to said gene, and
detecting specific hybridization.

173. A method of claim 172, wherein said detecting is performed by:
Northern blot analysis, polymerase chain reaction (PCR), reverse transcriptase PCR, RACE PCR, or in situ hybridization.

174. A method of detecting a pancreas cell, comprising:
contacting a sample comprising cells with a binding partner specific for a polypeptide coded for by TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127 of claim 199 under conditions effective for said binding partner bind specifically to said polypeptide, and
detecting specific binding.

175. A method of claim 174, wherein said detecting is performed by:
immunocytochemistry, immunoprecipitation, or Western blot.

176. A method of delivering an agent to a pancreas cell, comprising:
contacting a pancreas with an agent coupled to binding partner specific for TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127 of claim 199, whereby said agent is delivered to said cell.

177. A method of claim 176, wherein the agent is a therapeutic agent or an imaging agent.

178. A method of claim 176, wherein the agent is cytotoxic.

179. A method of claim 176, wherein the binding partner is an antibody.

180. A method of modulating a pancreas, immune, or reticuloendothelial cell, comprising:
contacting said cell with an agent effective to modulate TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, or the biological activity of a polypeptide encoded thereby, of claim 199, whereby the cell is modulated.

181. A method of assessing pancreas function, comprising:
detecting a polypeptide coded for by TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127 of claim 199, or fragments thereof, in a body fluid, whereby the amount of said polypeptide in said fluid is a measure of pancreas function.

182. A method of claim 181, wherein said detecting is performed using an antibody which is specific for said polypeptide.

183. A method of claim 181, wherein said detecting is performed by RIA, ELSA, or Western blot.

184. A method of expressing a heterologous polynucleotide in pancreas cells, comprising:
expressing a nucleic acid construct in pancreas cell, said construct comprising a promoter sequence operably linked to said heterologous polynucleotide, wherein said promoter sequence is selected SEQ ID NO 258, 261, 262, 265-267, 270-272, 275, 278, 279, 282-284, 287, 290-293, 296, 297, 300, 303, 306, 309-314, 317-320, 323-326, 329, 332-333, 336-338, 341, and 344.

185. A method of assessing a therapeutic or preventative intervention in a subject having a pancreas or lymphoid disease, comprising,
determining the expression levels of TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, or a polypeptide encoded thereby, of claim 199 in a tissue sample comprising pancreas, lymphoid, or reticuloendothelial cells.

186. A method of claim 185, further comprising assessing the expression levels of a plurality of said genes or polypeptides.

187. A method for identifying an agent that modulates the expression of TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, comprising,
contacting a pancreas, lymphoid, or reticuloendothelial cell, with a test agent under conditions effective for said test agent to modulate the expression of TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, of claim 199, and
determining whether said test agent modulates said gene.

188. A method of claim 187, wherein said agent is an antisense which is effective to inhibit translation of said gene.

189. A method for identifying an agent that modulates the expression of a polypeptide coded for by TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, comprising,
contacting a polypeptide coded for by TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127 of claim 199, with a test agent under conditions effective for said test agent to modulate said polypeptide, and
determining whether said test agent modulates said polypeptide.

190. A method of claim 189, wherein said test agent is an antibody.

191-197. (canceled)

198. A method of selecting a gene predominantly expressed in pancreas tissue from a database comprising polynucleotide and amino acid sequences for genes, comprising:
displaying, in a computer-readable medium, a polynucleotide sequence or polypeptide sequence for TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127, of claim 199, or complements to the polynucleotides sequence, wherein said displayed sequences have been retrieved from said database upon selection by a user.

199. A composition comprising genes and/or polypeptide which are expressed predominantly in pancreas tissue comprising: TMD0077, TMD0233, TMD0256, TMD0258, TMD0267, TMD0271, TMD0290, TMD0530, TMD0574, TMD0608, TMD0639, TMD0645, TMD0674, TMD0675, TMD0677, TMD0726, TMD0727, TMD0739, TMD0753, TMD1111, and/or TMD1127.

200. A method of treating pancreas cancer comprising,
administering an antibody specific for TMD 0645 which is effective to treat the pancreas cancer.
